(19) United States
(12) Patent Application Publication
(10) Pub. No.: US 2024/0059790 A1
(43) Pub. Date: Feb. 22, 2024

(54) COMPOSITIONS AND METHODS RELATED TO TUMOR ACTIVATED ANTIBODIES TARGETING PSMA AND EFFECTOR CELL ANTIGENS

(71) Applicant: Janux Therapeutics, Inc., San Diego, CA (US)

(72) Inventors: David CAMPBELL, San Diego, CA (US); Thomas R. DIRAIMONDO, San Diego, CA (US); Carolina CAFFARO, San Diego, CA (US); Hans AERNI, San Diego, CA (US)

(21) Appl. No.: 18/314,077

(22) Filed: May 8, 2023

Related U.S. Application Data
(63) Continuation of application No. PCT/CN2023/000050, filed on Apr. 4, 2023.
(60) Provisional application No. 63/338,220, filed on May 4, 2022.
(60) Provisional application No. 63/327,322, filed on Apr. 4, 2022.

(51) Int. Cl. C07K 16/30; A61P 35/00; C07K 16/28

(52) U.S. Cl. CPC C07K 16/3069; A61P 35/00; C07K 16/2809; C07K 2317/622

(57) ABSTRACT

Described herein are compositions and formulations comprising a recombinant polypeptide. Related methods and uses of these compositions and formulations are also described herein.

### CROSS-REFERENCE

This application is a continuation of International Application No. PCT/CN2023/000050, filed Apr. 4, 2023, which claims the benefit of U.S. Provisional Application No. 63/327,322, filed Apr. 4, 2022, and U.S. Provisional Application No. 63/338,220, filed May 4, 2022, each of which is incorporated herein by reference in its entirety.

### SEQUENCE LISTING

The instant application contains a Sequence Listing which has been submitted electronically in XML file format and is hereby incorporated by reference in its entirety. Said XML copy, created on Mar. 24, 2023, is named 52426-743_301 SL.xml and is 24,891 bytes in size.

### SUMMARY

Disclosed herein, in one aspect, is an isolated recombinant polypeptide complex comprising a first chain with an amino acid sequence having at least 80% sequence identity to SEQ ID NO: 1 and a second chain with an amino acid sequence having at least 80% sequence identity to SEQ ID NO: 2 wherein the isolated recombinant polypeptide complex comprises at least one of the following characteristics: (a) at least one disulfide bond formed by a pair of cysteine residues in the first chain or the second chain or by a pair of cysteine residues in the first chain and the second chain; (b) a secondary structure composition comprising a β-sheet or a random coil; (c) at least one pyroglutamine in the second chain; (d) a melting temperature (Tm) between of about 65° C. to about 85° C. when the isolated recombinant polypeptide complex is formulated at a concentration of 1.0 mg/mL in a buffer comprising 10 mM histidine buffer, 8% (w/v) Sucrose, 0.01% (w/v) polysorbate 20, pH 6.3; (e) afar UV circular dichroism peak at a wavelength between 190 nm and 205 nm when the isolated recombinant polypeptide complex is formulated at a concentration of 0.1 mg/mL in 10 mM potassium phosphate buffer pH 7.0; (f) a far UV circular dichroism dip at a wavelength between 210 nm and 220 nm when the isolated recombinant polypeptide complex is formulated at a concentration of 0.1 mg/mL in 10 mM potassium phosphate buffer pH 7.0; or (g) a near UV circular dichroism peak at a wavelength between 250 nm and 300 nm when the isolated recombinant polypeptide complex is formulated at a concentration of 1.0 mg/mL in a buffer comprising 10 mM histidine buffer, 8% (w/v) Sucrose, 0.01% (w/v) polysorbate 20, pH 6.3. In embodiments, the polypeptide comprises at least two of the characteristics. In some embodiments, the polypeptide comprises at least three of the characteristics. In some embodiments, the polypeptide comprises at least four of the characteristics. In some embodiments, the polypeptide comprises at least five of the characteristics. In some embodiments, the polypeptide comprises at least six of the characteristics. In some embodiments, the polypeptide comprises at least seven of the characteristics. In some embodiments, the polypeptide comprises at least eight of the characteristics. In some embodiments, the first chain comprises at least 85% sequence identity to SEQ ID NO: 1. In some embodiments, the first chain comprises at least 90% sequence identity to SEQ ID NO: 1. In some embodiments, the first chain comprises at least 95% sequence identity to SEQ ID NO: 1. In some embodiments, the first chain comprises at least 99% sequence identity to SEQ ID NO: 1. In some embodiments, the first chain comprises the amino acid sequence according to SEQ ID NO: 1. In some embodiments, the second chain comprises at least 85% sequence identity to SEQ ID NO: 2. In some embodiments, the second chain comprises at least 90% sequence identity to SEQ ID NO: 2. In some embodiments, the second chain comprises at least 95% sequence identity to SEQ ID NO: 2. In some embodiments, the second chain comprises at least 99% sequence identity to SEQ ID NO: 2. In some embodiments, the second chain comprises the amino acid sequence according to SEQ ID NO: 2.

In some embodiments, the at least one disulfide bond is an intra-chain disulfide bond formed between Cysteine 22 and Cysteine 96, between Cysteine 138 and Cysteine 148, between Cysteine 199 and Cysteine 275, between Cysteine 339 and Cysteine 407, between Cysteine 454 and Cysteine 519, or between Cysteine 565 and Cysteine 625 of the first chain. In some embodiments, the at least one disulfide bond is an intra-chain disulfide bond formed by a pair of cysteine residues of the second chain between Cysteine 22 and Cysteine 96, or between cysteine 150 and cysteine 206 of the second chain. In some embodiments, the at least one disulfide bond is an inter-chain disulfide bond formed between the first chain and the second chain between Cysteine 645 of the first chain and Cysteine 226 of the second chain. In some embodiments, the polypeptide comprises at least two disulfide bonds formed by pairs of cysteine residues. In some embodiments, the polypeptide comprises at least three disulfide bonds formed by pairs of cysteine residues. In some embodiments, the polypeptide comprises at least four disulfide bonds formed by pairs of cysteine residues. In some embodiments, the polypeptide comprises at least five disulfide bonds formed by pairs of cysteine residues. In some embodiments, the polypeptide comprises at least six disulfide bonds formed by pairs of cysteine residues. In some embodiments, the polypeptide comprises at least seven disulfide bonds formed by pairs of cysteine residues. In some embodiments, the polypeptide comprises at least eight disulfide bonds formed by pairs of cysteine residues. In some embodiments, the pair of cysteine residues comprises Cysteine 22 and Cysteine 96 of SEQ ID NO: 1. In some embodiments, the pair of cysteine residues comprises Cysteine 138 and Cysteine 148 of SEQ ID NO: 1. In some embodiments, the pair of cysteine residues comprises Cysteine 199 and Cysteine 275 of SEQ ID NO: 1. In some embodiments, the pair of cysteine residues comprises Cysteine 339 and Cysteine 407 of SEQ ID NO: 1. In some embodiments, the pair of cysteine residues comprises Cysteine 454 and Cysteine 519 of SEQ ID NO: 1. In some embodiments, the pair of cysteine residues comprises Cysteine 565 and Cysteine 625 of SEQ ID NO: 1. In some embodiments, the pair of cysteine residues comprises Cysteine 645 of SEQ ID NO: 1 and Cysteine 226 of SEQ ID NO: 2. In some embodiments, the pair of cysteine residues comprises Cysteine 150 and Cysteine 206 of SEQ ID NO: 2. In some embodiments, the pair of cysteine residues comprises Cysteine 22 and Cysteine 96 of SEQ ID NO: 2. In some embodiments, at least one cysteine residue is a free sulfhydryl. In some embodiments, at least one cysteine residues is a free sulfhydryl, and at least one cysteine is selected from and corresponding to Cysteine 22, Cysteine 96, Cysteine 138, Cysteine 148, Cysteine 199, Cysteine 275, Cysteine 339, Cysteine 407, Cysteine 454, Cysteine 519, Cysteine 565, Cysteine 625, and Cysteine 645 of SEQ ID NO: 1. In some embodiments, at least one cysteine residues is a free sulfhydryl, and the at least one cysteine is selected from selected from and corresponding to Cysteine 22, Cysteine 96, Cysteine 150, Cysteine 226 and Cysteine 206 of SEQ ID NO: 2. In some embodiments, the at least one pair of cysteine residues comprises Cysteine 22 and Cysteine 96 of SEQ ID NO: 1, Cysteine 138 and Cysteine 148 of SEQ ID NO: 1, Cysteine 199 and Cysteine 275 of SEQ ID NO: 1, Cysteine 454 and Cysteine 519 of SEQ ID NO: 1, Cysteine 565 and Cysteine 625 of SEQ ID NO: 1, Cysteine 22 and Cysteine 96 of SEQ ID NO: 2, Cysteine 150 and Cysteine 206 of SEQ ID NO: 2, and Cysteine 645 of SEQ ID NO: 1 and Cysteine 226 of SEQ ID NO: 2.

In some embodiments, the secondary structure composition comprises a β-sheet and a random coil. In some embodiments, the isolated recombinant polypeptide complex has a melting temperature (Tm) between of about 71° C. to about 81° C. In some embodiments, the isolated recombinant polypeptide complex has a melting temperature (Tm) between of about 71.4° C. to about 79.5° C. In some embodiments, the isolated recombinant polypeptide complex has a melting temperature (Tm) between of about 65° C. to about 85° C. In some embodiments, the isolated recombinant polypeptide complex has a far UV circular dichroism peak at a wavelength less than or equal to 195 nm. In some embodiments, the isolated recombinant polypeptide complex has a far UV circular dichroism peak at a wavelength less than or equal to 205 nm. In some embodiments, the isolated recombinant polypeptide complex has a far UV circular dichroism dip at a wavelength less than or equal to 220 nm. In some embodiments, the isolated recombinant polypeptide complex has a near UV circular dichroism peak at a wavelength less than or equal to 273 nm. In some embodiments, the isolated recombinant polypeptide complex has a near UV circular dichroism peak at a wavelength less than or equal to 279 nm. In some embodiments, the isolated recombinant polypeptide complex has a near UV circular dichroism peak at a wavelength less than or equal to 290 nm. In some embodiments, the isolated recombinant polypeptide complex has a near UV circular dichroism peak at a wavelength less than or equal to 295 nm.

Disclosed herein, in another aspect, is a method of treating cancer comprising administering to a subject in need thereof an isolated recombinant polypeptide complex disclosed herein.

Disclosed herein, in another aspect, is a plurality of isolated recombinant polypeptide complexes comprising a first chain with an amino acid sequence having at least 80% sequence identity to SEQ ID NO: 1 and a second chain with an amino acid sequence having at least 80% sequence identify to SEQ ID NO: 2, wherein the plurality comprises greater than 90% monomer of the isolated recombinant polypeptide complexes. In some embodiments, the plurality of isolated recombinant polypeptide complexes comprise greater than 95% monomer. In some embodiments, the plurality of isolated recombinant polypeptide complexes comprise greater than 99% monomer. In some embodiments, the plurality of isolated recombinant polypeptide complexes comprise a concentration greater than or equal to 1.0 mg/mL. In some embodiments, the plurality of isolated recombinant polypeptide complexes comprise a concentration of at least 2.0 mg/mL. In some embodiments, the plurality of isolated recombinant polypeptide complexes comprise a pH greater than or equal to 5.0. In some embodiments, the plurality of isolated recombinant polypeptide complexes comprise greater than 90% monomer at a concentration of greater than or equal to 2.0 mg/mL and a pH of 6.3.

Disclosed herein, in another aspect, is a method for treating prostate cancer comprising administering to a subject in need thereof an antibody or antigen binding fragment that comprises a CD3 binding domain and a PSMA binding domain wherein the antibody or antibody binding fragment is administered to the subject once weekly at a dose of at least 0.1 μg/kg. In some embodiments, the antibody or antigen binding fragment is administered to the subject at a dose of at least 0.1 mg/kg. In some embodiments, the antibody or antigen binding fragment is administered to the subject at a dose of at least 0.3 mg/kg. In some embodiments, the antibody or antigen binding fragment is administered to the subject at a dose of at least 1.0 mg/kg. In some embodiments, the antibody or antigen binding fragment is administered to the subject at a dose of at least 1.5 mg/kg. In some embodiments, the antibody or antigen binding fragment is administered to the subject according to the following treatment regimen: administration of the antibody or antigen binding fragment to the subject at a dose of at least 0.1 μg/kg the first week, administration of the antibody or antigen binding fragment to the subject at a dose of at least 0.3 mg/kg the second week, administration of the antibody or antigen binding fragment to the subject at a dose of at least 1.5 mg/kg the third week, followed by a 4 week non-treatment interval of the antibody or antigen binding fragment.

In some embodiments, the isolated recombinant polypeptide complex provides a maximum plasma concentration (Cmax) in a subject within about 0.1 hour after intravenous administration. In some embodiments, the isolated recombinant polypeptide complex provides a Cmax of about 2500 to 3500 ng/ml in a subject within about 0.1 hour after intravenous administration of a dose of about 0.1 mg/kg. In some embodiments, the isolated recombinant polypeptide complex provides a Cmax of about 7500 to 10500 ng/ml in a subject within about 0.1 hour after intravenous administration of a dose of about 0.3 mg/kg. In some embodiments, the isolated recombinant polypeptide complex provides a Cmax of about 37500 to 52500 ng/ml in a subject within about 0.1 hour after intravenous administration of a dose of about 1.5 mg/kg. In some embodiments, the isolated recombinant polypeptide complex provides a half maximum plasma concentration in a subject in about 60 to about 120 hours (T1/2) after intravenous administration. In some embodiments, the value of Cmax correlates with the dose of the isolated recombinant polypeptide complex that is administered. In some embodiments, the value of Cmax is proportional to the dose of the isolated recombinant polypeptide complex that is administered. In some embodiments, less than 1% of the isolated recombinant polypeptide complex degrades each day in the serum of a subject. In some embodiments, the subject is diagnosed with cancer. In some embodiments, the cancer comprises prostate cancer. In some embodiments, the cancer comprises metastatic castration-resistant prostate cancer (mCRPC).

Disclosed herein, in another aspect, is a method for treating cancer comprising administering to a subject in need thereof an effective amount of an isolated recombinant polypeptide complex disclosed herein. In some embodiments, the method comprises administering the isolated recombinant polypeptide complex to the subject at a first dose and a second dose, wherein the second dose is equal to or higher than the first dose, wherein the first or second dose is at least 100 μg. In some embodiments, the method comprises administering the isolated recombinant polypeptide complex to the subject at a first dose, a second dose and a third dose, wherein the second dose is equal to or higher than the first dose and the third dose is equal to or higher than the second dose, wherein the first, second, or third dose is at least 100 μg. In some embodiments, the method comprises a first treatment course and a second treatment course, wherein the first dose is administered to the subject in the first treatment course, wherein the second dose is administered to the subject in the second treatment course. In some embodiments, the method comprises a 21-day treatment course, wherein the first dose is administered to the subject in the first week of the treatment course, wherein the second dose is administered to the subject in the second week of the treatment course, wherein the third dose is administered to the subject in the third week of the treatment course. In some embodiments, the method comprises a first 21-day treatment course and a second 21-day treatment course, wherein the first dose of the second 21-day treatment course is equal to or higher than the first dose of the first 21-day treatment course, wherein the second dose of the second 21-day treatment course is equal to or higher than the second dose of the first 21-day treatment course, wherein the third dose of the second 21-day treatment course is equal to or higher than the third dose of the first 21-day treatment course. In some embodiments, the method comprises a 21-day treatment cycle comprising: (a) administering the isolated recombinant polypeptide complex to the subject at a first dose in the first week of the treatment cycle; (b) administering the isolated recombinant polypeptide complex to the subject at a second dose in the second week of the treatment cycle; and (c) administering the isolated recombinant polypeptide complex to the subject at a third dose in the third week of the treatment cycle. In some embodiments, the method comprises: (a) administering the isolated recombinant polypeptide complex to the subject at the first dose on the first day of the 21-day treatment cycle; (b) administering the isolated recombinant polypeptide complex to the subject at the second dose on the eighth day of the 21-day treatment cycle; and (c) administering the isolated recombinant polypeptide complex to the subject at the third dose on the fifteenth day of the 21-day treatment cycle. In some embodiments, the treatment cycle is repeated 6 times over 18 weeks. In some embodiments, the first, second, or third dose is at least 100 μg. In some embodiments, the second dose is equal to or higher than the first dose. In some embodiments, the third dose is equal to or higher than the second dose. In some embodiments, the third dose is equal to or higher than the first dose. In some embodiments, the method comprises a first 21-day treatment cycle and a second 21-day treatment cycle, wherein the first dose of the second 21-day treatment cycle is equal to or higher than the first dose of the first 21-day treatment cycle, wherein the second dose of the second 21-day treatment cycle is equal to or higher than the second dose of the first 21-day treatment cycle, wherein the third dose of the second 21-day treatment cycle is equal to or higher than the third dose of the 21-day first treatment cycle. In some embodiments, the administering comprises administering through intravenous infusion. In some embodiments, the cancer comprises prostate cancer. In some embodiments, the cancer comprises mCRPC. In some embodiments, the method further comprises treating the subject with a therapy for an infusion-related reaction before the administering. In some embodiments, the therapy for an infusion-related reaction comprises an antipyretic drug, an antihistamine drug, an antiemetic drug, or a corticosteroid. In some embodiments, the therapy for an infusion-related reaction comprises acetaminophen, paracetamol, or diphenhydramine. In some embodiments, the method further comprises treating the subject with a corticosteroid before the step (a). In some embodiments, the method further comprises treating the subject with a corticosteroid before the step (a). In some embodiments, the method further comprises treating the subject with a therapy for cytokine release syndrome (CRS) before or after the administering. In some embodiments, the therapy for CRS comprises an intravenous hydration procedure, an oxygen treatment, a corticosteroid, an immunosuppressant, a vasopressor, or an antiepileptic drug. In some embodiments, the oxygen treatment comprises mechanical ventilation. In some embodiments, the immunosuppressant comprises an IL-6 receptor inhibitor. In some embodiments, the IL-6 receptor inhibitor comprises tocilizumab. In some embodiments, the therapy for CRS comprises a high dose of a corticosteroid. In some embodiments, the isolated recombinant polypeptide complex is cleaved by a tumor specific protease to generate an enzymatic product of the isolated recombinant polypeptide complex after the administering. In some embodiments, the tumor specific protease comprises two or more proteases, wherein the isolated recombinant polypeptide complex is cleaved by a first protease of the two or more proteases to generate a first metabolic product of the isolated recombinant polypeptide complex, wherein the isolated recombinant polypeptide complex is cleaved by a second protease of the two or more proteases to generate a second metabolic product of the isolated recombinant polypeptide complex. In some embodiments, the first protease comprises a serine protease, wherein the second protease comprises a matrix metalloprotease. In some embodiments, the serine protease comprises recombinant human matriptase (MTSP1), wherein the matrix metalloprotease comprises recombinant human matrix metalloprotease 9 (MMP9). In some embodiments, the first metabolic product comprises an amino acid sequence with at least 90% sequence identity to the amino acid sequence of SEQ ID NO: 5. In some embodiments, the second metabolic product comprises an amino acid sequence with at least 90% sequence identity to the amino acid sequence of SEQ ID NO: 6. In some embodiments, the subject is a non-human primate or human. In some embodiments, the non-human primate is cynomolgus monkey.

Disclosed herein, in another aspect, is a pharmaceutical composition comprising: (a) a isolated recombinant polypeptide complex disclosed herein; and (b) a pharmaceutically acceptable excipient. In some embodiments, the pharmaceutically acceptable excipient comprises a buffer, a stabilizing agent, a tonicity agent, a surfactant, or combinations thereof. In some embodiments, the buffer comprises an amino acid or a derivative thereof. In some embodiments, the amino acid or the derivative thereof comprises L-histidine, L-histidine monohydrochloride monohydrate, or combinations thereof. In some embodiments, the stabilizing agent comprises sugar. In some embodiments, the sugar comprises sucrose. In some embodiments, the tonicity agent comprises sugar. In some embodiments, the sugar comprises sucrose. In some embodiments, the surfactant comprises polysorbate 20. In some embodiments, the total amount of L-histidine in the pharmaceutical composition is about 10 mM in the forms of both L-histidine and L-histidine monohydrochloride monohydrate. In some embodiments, the molar ratio of L-histidine to L-histidine monohydrochloride monohydrate is about 3:2. In some embodiments, the pharmaceutical composition comprises about 8% (w/v) sucrose. In some embodiments, the pharmaceutical composition comprises at least 0.01% (w/v) polysorbate 20. In some embodiments, the pharmaceutical composition comprises about 6 mM L-histidine, about 4 mM L-histidine monohydrochloride monohydrate, about 8% (w/v) sucrose, and about 0.01% (w/v) polysorbate 20. In some embodiments, the pharmaceutical composition comprises about 2 mg/ml of the isolated recombinant polypeptide complex. In some embodiments, the pharmaceutical composition comprises a pH between about 5 and about 7. In some embodiments, the pharmaceutical composition comprises a pH of about 6.3.

Disclosed herein, in another aspect, is an isolated polypeptide comprising an amino acid sequence with at least 90% sequence identity to the amino acid sequence of SEQ ID NO: 5 and wherein the isolated polypeptide is 484 amino acids in length.

Disclosed herein, in another aspect, is an isolated polypeptide comprising an amino acid sequence with at least 90% sequence identity to the amino acid sequence of SEQ ID NO: 6 and wherein the isolated polypeptide is 476 amino acids in length.

Disclosed herein, in another aspect, is a pharmaceutical composition comprising an isolated polypeptide comprising an amino acid sequence with at least 90% sequence identity to the amino acid sequence of SEQ ID NO: 5 and wherein the isolated polypeptide is 484 amino acids in length, and pharmaceutically acceptable excipients.

Disclosed herein, in another aspect, is a pharmaceutical composition comprising an isolated polypeptide comprising an amino acid sequence with at least 90% sequence identity to the amino acid sequence of SEQ ID NO: 6 and wherein the isolated polypeptide is 476 amino acids in length and pharmaceutically acceptable excipients.

Disclosed herein, in another aspect, is a method for treating cancer comprising administering to a subject in need thereof an effective amount of the pharmaceutical composition disclosed herein. In some embodiments, the cancer comprises mCRPC. In some embodiments, the subject is human.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. The patent or application file contains at least one drawing executed in color. Copies of this patent or patent application publication with color drawing(s) will be provided by the Office upon request and payment of the necessary fee. A better understanding of the features and advantages of the present invention will be obtained by reference to the following detailed description that sets forth illustrative embodiments, in which the principles of the disclosure are utilized, and the accompanying drawings (also “Figure” and “FIG.” herein), of which:

FIG. 1 illustrates design, structure and mechanism of action of polypeptide complex 1 (PC-1). PC-1 is a tumor-activated T cell engager with PSMA- and CD3-binding domains, an albumin-binding domain to extend circulating half-life, a peptide mask that inhibits CD3 engagement on T cells, and a tumor protease cleavable linker. Tumor-specific proteolysis of the cleavable linker in the tumor microenvironment (TME) separates the tandem mask and albumin-binding domain from PC-1. It enables TME restricted CD3 binding and subsequent T cell activation against PSMA expressing prostate cancer cells. Loss of the albumin-binding domain likely ensures that any activated PC-1 that migrates out of the tumor will be cleared rapidly and reduces its potential accumulation in healthy tissues that can contribute to safety risks.

FIGS. 2A-2D illustrate mask discovery by peptide phage display. Phage displaying peptide libraries were screened for binding to surface-immobilized anti-CD3 scFv. After several bind, elute, and amplify cycles, clonal phages were screened for CD3 competitive binding by ELISA. Selected clonal phage sequences were synthesized as peptides and screened for binding and inhibition properties against anti-CD3 scFv. Peptide inhibitors were then incorporated into polypeptide complex designs.

FIGS. 3A-3F illustrate binding of PC-1 to CD3 is cleavage and dose-dependent. PC-1 CD3 target engagement is cleavage dependent where masking reduces CD3 binding by >600 fold. Treatment of PC-1 with protease enzyme enables potent CD3 binding comparable to non-masked TCE-1. PC-1 exhibits potent binding to human and monkey PSMA and albumin.

FIGS. 4A-4B illustrate TCE-1 potency depends on structure and orientation. TCE-1 activity depends on the connecting geometry of PSMA and CD3 binding domains.

FIGS. 5A-5F illustrate activity of PC-1 is cleavage, dose- and PSMA expression-dependent. Masking of the PC-1 CD3-binding domain reduces its capacity to induce cytokine release. Functional activity in prostate cancer and T cell co-culture assays is dependent on masking and PSMA expression. PC-1 demonstrates a robust T cell functional shift that demonstrates potential for an enhanced safety profile.

FIGS. 6A-6C illustrate PC-1 has extended half-life and enhanced safety profile in NHPs.

FIG. 7 illustrates a flow diagram of an upstream cell culture process involved in the production of PC-1.

FIG. 8 illustrates a flow diagram of a downstream purification process involved in the production of PC-1.

FIG. 9 illustrates circular dichroism (CD) spectra of PC-1 in the far-UV region.

FIG. 10 illustrates CD spectra of PC-1 in the near-UV region.

FIG. 11 illustrates differential scanning calorimetry data for PC-1.

FIGS. 12A-12B illustrate SEC-MALS chromatograms of PC-1.

FIG. 13 illustrates light and heavy chain arrangement in PC-1.

FIGS. 14A-14F illustrate ELISA Binding of Test Articles to PSMA.

FIGS. 15A-15D illustrate ELISA Binding of PC-1 to albumin.

FIGS. 16A-16F illustrate ELISA Binding of Test Articles to CD3.

FIGS. 17A-17B illustrate example of CD3 Octet binding data for PC-1 over time after incubation in serum.

FIGS. 18A-18H illustrate PC-1 cleavage rate in Cynomolgus monkey, healthy human, or human mCRPC donor serum.

FIGS. 19A-19I illustrate dose-dependent test-article induced production of IFNγ, TNF, and IL-6 by PBMCs cocultured with LNCaP tumor cells.

FIGS. 20A-20I illustrate dose-dependent test-article induced production of IFNγ, TNF, and IL-6 by PBMCs cocultured with 22Rv1 tumor cells.

FIGS. 21A-21F illustrates absence of test article-induced cytokine production by PBMCs in the absence of tumor cells.

FIGS. 22A-22H illustrate dose-dependent LNCaP tumor cell killing by test article-stimulated PBMCs.

FIGS. 23A-23H illustrate dose-dependent 22Rv1 tumor cell killing by test article-stimulated PBMCs.

FIGS. 24A-24D illustrates absence of PC3 tumor cell killing by test article-stimulated PBMCs.

FIG. 25 illustrates the samples preparation workflow using immunocapture purification.

FIG. 26 illustrates the TK profile of PC-1 in monkey plasma samples collected from day 22 Group 2 of the TK study

FIG. 27 illustrates the correlation between the intact PC-1 concentration and anti-drug antibody (ADA) concentration in the high-dose group of monkey.

FIGS. 28A-28D illustrate structures of PC-1, PC-1-SP cleaved, PC-1-MMP cleaved, and active PC-1-TCE.

FIG. 29 illustrates activation of PC-1 by serum proteases to yield active, non-masked PC-1-TCE.

### DETAILED DESCRIPTION

In the context of the present application, the following terms have the meanings ascribed to them unless specified otherwise:

As used throughout the specification and claims, the terms “a”, “an” and “the” are generally used in the sense that they mean “at least one”, “at least a first”, “one or more” or “a plurality” of the referenced components or steps, except in instances wherein an upper limit is thereafter specifically stated. For example, a “cleavage sequence”, as used herein, means “at least a first cleavage sequence” but includes a plurality of cleavage sequences. The operable limits and parameters of combinations, as with the amounts of any single agent, will be known to those of ordinary skill in the art in light of the present application.

The terms “polypeptide”, “peptide”, and “protein” are used interchangeably herein to generally refer to polymers of amino acids of any length. The polymer may be linear or branched, it may comprise modified amino acids, and it may be interrupted by non-amino acids. The terms also encompass an amino acid polymer that has been modified, for example, by disulfide bond formation, glycosylation, lipidation, acetylation, phosphorylation, or any other manipulation, such as conjugation with a labeling component.

As used herein in the context of the structure of a polypeptide, “N-terminus” (or “amino terminus”) and “C-terminus” (or “carboxyl terminus”) generally refer to the extreme amino and carboxyl ends of the polypeptide, respectively.

The term “therapeutically effective amount,” as used herein, generally means the amount of a polypeptide variant or a polypeptide composition that, when administered to a patient for treating a disease or other undesirable medical condition, is sufficient to have a beneficial effect with respect to that disease or condition. The therapeutically effective amount will vary depending on the polypeptide variant or the polypeptide composition, the disease or condition and its severity, and the age, weight, etc. of the patient to be treated. Determining the therapeutically effective amount of a given polypeptide variant or a given polypeptide composition is generally within the ordinary skill of the art and requires no more than routine experimentation.

As used herein, the terms “about” and “approximately” are used interchangeably. Any numerals used herein with or without about/approximately are meant to cover any normal fluctuations appreciated by one of ordinary skill in the relevant art. For example, the term “about” may refer to a range of values ±10%, ±5%, ±2%, or ±1% of a specified value. By way of an example, For example, the phrase “about 50%” may include from 45% to 55%, from 48% to 52%, or from 49% to 51%.

The above definitions supersede any conflicting definition in any reference that is incorporated by reference herein. The fact that certain terms are defined, however, should not be considered as indicative that any term that is undefined is indefinite. Rather, all terms used are believed to describe the disclosure in terms such that one of ordinary skill can appreciate the scope and practice the present application.

### Recombinant Polypeptide Compositions

Metastatic castration-resistant prostate cancer (mCRPC) remains an incurable disease. Bispecific T cell engagers (TCEs) targeting prostate-specific membrane antigen (PSMA) on prostate tumor cells and cluster of differentiation 3 (CD3) on T cells have clinical efficacy for the treatment of mCRPC. These TCEs have issues of cytokine release syndrome (CRS) and poor pharmacokinetic (PK) profile. There remains a need for new immune therapy. Disclosed herein is a PSMA-targeted tumor-activated T cell engager (TRACTr) featuring enhanced safety and pharmacokinetics profiles.

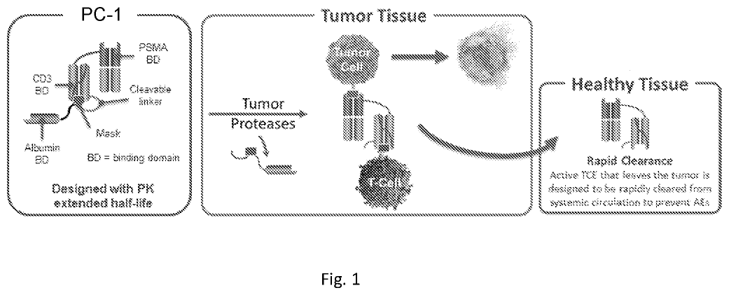

FIG. 1 illustrates design, structure and mechanism of action of polypeptide complex 1 (PC-1). PC-1 is a tumor-activated T cell engager with PSMA- and CD3-binding domains, an albumin-binding domain to extend circulating half-life, a peptide mask that inhibits CD3 engagement on T cells, and a tumor protease cleavable linker. Tumor-specific proteolysis of the cleavable linker in the tumor microenvironment (TME) separates the tandem mask and albumin-binding domain from PC-1. It enables TME restricted CD3 binding and subsequent T cell activation against PSMA expressing prostate cancer cells. Loss of the albumin-binding domain likely ensures that any activated PC-1 that migrates out of the tumor will be cleared rapidly and reduces its potential accumulation in healthy tissues that can contribute to safety risks.

Disclosed herein is a recombinant polypeptide. In some embodiments, the recombinant polypeptide comprises a tumor-activated T cell engager with PSMA- and CD3-binding domains, an albumin binding domain to extend circulating half-life, a peptide mask that inhibits CD3 engagement on T-cells, and a tumor protease cleavable linker. Tumor specific proteolysis of the cleavable linker in the tumor microenvironment can separate the tandem mask and albumin-binding domain from the recombinant polypeptide. The recombinant polypeptide can comprise chain 1 and chain 2 as described herein. In some embodiments, chain 1 comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 1. In some embodiments, chain 2 comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 2.

In some embodiments, the recombinant polypeptide comprises an amino acid sequence having at least 80% sequence identity to SEQ ID NO: 1. In some embodiments, the recombinant polypeptide comprises an amino acid sequence having at least 85% sequence identity to SEQ ID NO: 1. In some embodiments, the recombinant polypeptide comprises an amino acid sequence having at least 90% sequence identity to SEQ ID NO: 1. In some embodiments, the recombinant polypeptide comprises an amino acid sequence having at least 91% sequence identity to SEQ ID NO: 1. In some embodiments, the recombinant polypeptide comprises an amino acid sequence having at least 92% sequence identity to SEQ ID NO: 1. In some embodiments, the recombinant polypeptide comprises an amino acid sequence having at least 93% sequence identity to SEQ ID NO: 1. In some embodiments, the recombinant polypeptide comprises an amino acid sequence having at least 94% sequence identity to SEQ ID NO: 1. In some embodiments, the recombinant polypeptide comprises an amino acid sequence having at least 95% sequence identity to SEQ ID NO: 1. In some embodiments, the recombinant polypeptide comprises an amino acid sequence having at least 96% sequence identity to SEQ ID NO: 1. In some embodiments, the recombinant polypeptide comprises an amino acid sequence having at least 97% sequence identity to SEQ ID NO: 1. In some embodiments, the recombinant polypeptide comprises an amino acid sequence having at least 98% sequence identity to SEQ ID NO: 1. In some embodiments, the recombinant polypeptide comprises an amino acid sequence having at least 99% sequence identity to SEQ ID NO: 1.

In some embodiments, the recombinant polypeptide comprises an amino acid sequence according to SEQ ID NO: 1.

In some embodiments, the recombinant polypeptide comprises an amino acid sequence having at least 80% sequence identity to SEQ ID NO: 2. In some embodiments, the recombinant polypeptide comprises an amino acid sequence having at least 85% sequence identity to SEQ ID NO: 2. In some embodiments, the recombinant polypeptide comprises an amino acid sequence having at least 90% sequence identity to SEQ ID NO: 2. In some embodiments, the recombinant polypeptide comprises an amino acid sequence having at least 91% sequence identity to SEQ ID NO: 2. In some embodiments, the recombinant polypeptide comprises an amino acid sequence having at least 92% sequence identity to SEQ ID NO: 2. In some embodiments, the recombinant polypeptide comprises an amino acid sequence having at least 93% sequence identity to SEQ ID NO: 2. In some embodiments, the recombinant polypeptide comprises an amino acid sequence having at least 94% sequence identity to SEQ ID NO: 2. In some embodiments, the recombinant polypeptide comprises an amino acid sequence having at least 95% sequence identity to SEQ ID NO: 2. In some embodiments, the recombinant polypeptide comprises an amino acid sequence having at least 96% sequence identity to SEQ ID NO: 2. In some embodiments, the recombinant polypeptide comprises an amino acid sequence having at least 97% sequence identity to SEQ ID NO: 2. In some embodiments, the recombinant polypeptide comprises an amino acid sequence having at least 98% sequence identity to SEQ ID NO: 2. In some embodiments, the recombinant polypeptide comprises an amino acid sequence having at least 99% sequence identity to SEQ ID NO: 2.

In some embodiments, the recombinant polypeptide comprises an amino acid sequence according to SEQ ID NO: 2.

TABLE 1
Recombinant Polypeptide Amino Acid Sequences

| Construct | Amino Acid Sequence | |
|---|---|---|
| Description | (N to C) | SEQ ID NO: |
| Chain 1 | EVQLVESGGGLVQPGGSLRLSCAASGSTFYTAVMGWV | 1 |
| PC-1 | RQAPGKGLEWVAAIRWTALTTSYADSVKGRFTISRDGA | |
| | KTTLYLQMNSLRPEDTAVYYCAARGTLGLFTTADSYD | |
| | YWGQGTLVTVSSGGGGSGGGSGGVYCGPEFDESVGCM | |
| | GGGGSGGGLSGRSDAGSPLGLAGSGGGSEVQLVESGG | |
| | GLVQPGGSLKLSCAASGFTFNKYAMNWVRQAPGKGLE | |
| | WVARIRSKYNNYATYYADSVKDRFTISRDDSKNTAYL | |
| | QMNNLKTEDTAVYYCVRHGNFGNSYISYWAYWGQGT | |
| | LVTVSSGGGGSGGGGSGGGGSQTVVTQEPSLTVSPGGT | |
| | VTLTCGSSTGAVTSGNYPNWVQQKPGQAPRGLIGGTKF | |
| | LAPGTPARFSGSLLGGKAALTLSGVQPEDEAEYYCVLW | |
| | YSNRWVFGGGTKLTVLGGGGSDIQMTQSPSSLSASVGD | |
| | RVTITCRASQGISNYLAWYQQKTGKVPKFLIYEASTLOS | |
| | GVPSRFSGGGSGTDFTLTISSLQPEDVATYYCQNYNSAP | |
| | FTFGPGTKVDIKRTVAAPSVFIFPPSDEQLKSGTASVVCL | |
| | LNNFYPREAKVQWKVDNALQSGNSQESVTEQDSKDST | |
| | YSLSSTLTLSKADYEKHKVYACEVTHQGLSSPVTKSFN | |
| | RGEC | |
| Chain 2 | QVQLVESGGGVVQPGRSLRLSCAASGFAFSRYGMHW | 2 |
| PC-1 | VRQAPGKGLEWVAVIWYDGSNKYYADSVKGRFTISRD | |
| | NSKNTQYLQMNSLRAEDTAVYYCARGGDFLYYYYYG | |
| | MDVWGQGTTVTVSSASTKGPSVFPLAPSSKSTSGGTAA | |
| | LGCLVKDYFPEPVTVSWNSGALTSGVHTFPAVLQSSGL | |
| | YSLSSVVTVPSSSLGTQTYICNVNHKPSNTKVDKKVEPK | |
| | SC | |
| Chain 3 | EVOLVESGGGLVQPGGSLKLSCAASGFTFNKYAMNW | 3 |
| TCE-1 | VRQAPGKGLEWVARIRSKYNNYATYYADSVKDRFTIS | |
| | RDDSKNTAYLQMNNLKTEDTAVYYCVRHGNFGNSYIS | |
| | YWAYWGQGTLVTVSSGGGGSGGGGSGGGGSQTVVTQ | |
| | EPSLTVSPGGTVTLTCGSSTGAVTSGNYPNWVQQKPGQ | |
| | APRGLIGGTKFLAPGTPARFSGSLLGGKAALTLSGVQPE | |
| | DEAEYYCVLWYSNRWVFGGGTKLTVLGGGGSDIQMT | |
| | QSPSSLSASVGDRVTITCRASQGISNYLAWYQQKTGKV | |
| | PKFLIYEASTLQSGVPSRFSGGGSGTDFTLTISSLQPEDV | |
| | ATYYCONYNSAPFTFGPGTKVDIKRTVAAPSVFIFPPSD | |
| | EQLKSGTASVVCLLNNFYPREAKVQWKVDNALQSGNS | |
| | QESVTEQDSKDSTYSLSSTLTLSKADYEKHKVYACEVT | |
| | HQGLSSPVTKSFNRGEC | |
| Chain 4 | QVQLVESGGGVVQPGRSLRLSCAASGFAFSRYGMHW | 4 |
| TCE-1 | VRQAPGKGLEWVAVIWYDGSNKYYADSVKGRFTISRD | |
| | NSKNTQYLQMNSLRAEDTAVYYCARGGDFLYYYYYG | |
| | MDVWGQGTTVTVSSASTKGPSVFPLAPSSKSTSGGTAA | |
| | LGCLVKDYFPEPVTVSWNSGALTSGVHTFPAVLQSSGL | |
| | YSLSSVVTVPSSSLGTQTYICNVNHKPSNTKVDKKVEPK | |
| | SC | |
| Metabolite 1 | SDAGSPLGLAGSGGGSEVQLVESGGGLVQPGGSLKLSC | 5 |
| PC-1 | AASGFTFNKYAMNWVRQAPGKGLEWVARIRSKYNNY | |
| | ATYYADSVKDRFTISRDDSKNTAYLQMNNLKTEDTAV | |
| | YYCVRHGNFGNSYISYWAYWGQGTLVTVSSGGGGSGG | |
| | GGSGGGGSQTVVTQEPSLTVSPGGTVTLTCGSSTGAVT | |
| | SGNYPNWVQQKPGQAPRGLIGGTKFLAPGTPARFSGSL | |
| | LGGKAALTLSGVQPEDEAEYYCVLWYSNRWVFGGGT | |
| | KLTVLGGGGSDIQMTQSPSSLSASVGDRVTITCRASQGI | |
| | SNYLAWYQQKTGKVPKFLIYEASTLQSGVPSRFSGGGS | |
| | GTDFTLTISSLQPEDVATYYCQNYNSAPFTFGPGTKVDI | |
| | KRTVAAPSVFIFPPSDEQLKSGTASVVCLLNNFYPREAK | |
| | VQWKVDNALQSGNSQESVTEQDSKDSTYSLSSTLTLSK | |
| | ADYEKHKVYACEVTHQGLSSPVTKSFNRGEC | |
| Metabolite 2 | LAGSGGGSEVQLVESGGGLVQPGGSLKLSCAASGFTFN | 6 |
| PC-1 | KYAMNWVRQAPGKGLEWVARIRSKYNNYATYYADSV | |
| | KDRFTISRDDSKNTAYLQMNNLKTEDTAVYYCVRHGN | |
| | FGNSYISYWAYWGQGTLVTVSSGGGGSGGGGSGGGGS | |
| | QTVVTQEPSLTVSPGGTVTLTCGSSTGAVTSGNYPNWV | |
| | QQKPGQAPRGLIGGTKFLAPGTPARFSGSLLGGKAALTL | |
| | SGVQPEDEAEYYCVLWYSNRWVFGGGTKLTVLGGGGS | |
| | DIQMTQSPSSLSASVGDRVTITCRASQGISNYLAWYQQK | |
| | TGKVPKFLIYEASTLQSGVPSRFSGGGSGTDFTLTISSLQ | |
| | PEDVATYYCQNYNSAPFTFGPGTKVDIKRTVAAPSVFIF | |
| | PPSDEQLKSGTASVVCLLNNFYPREAKVQWKVDNALQ | |
| | SGNSQESVTEQDSKDSTYSLSSTLTLSKADYEKHKVYA | |
| | CEVTHQGLSSPVTKSFNRGEC | |

### Disulfide Bond(s)

In some embodiments, the recombinant polypeptide comprises at least one disulfide bond formed by a pair of cysteine residues. In some embodiments, the recombinant polypeptide comprises at least one disulfide bond formed by a pair of cysteine residues in the first chain. In some embodiments, the recombinant polypeptide comprises at least one disulfide bond formed by a pair of cysteine residues in the second chain. In some embodiments, the recombinant polypeptide comprises at least one disulfide bond formed by a pair of cysteine residues in the first chain and the second chain. In some embodiments, the recombinant polypeptide comprises at least two, at least three, or at least four disulfide bonds formed by pairs of cysteine residues comprising the pair of cysteine residues. In some embodiments, the recombinant polypeptide comprises at least two disulfide bonds formed by pairs of cysteine residues comprising the pair of cysteine residues. In some embodiments, the recombinant polypeptide comprises at least three disulfide bonds formed by pairs of cysteine residues comprising the pair of cysteine residues. In some embodiments, the recombinant polypeptide comprises at least four disulfide bonds formed by pairs of cysteine residues comprising the pair of cysteine residues. In some embodiments, the recombinant polypeptide comprises at least five disulfide bonds formed by pairs of cysteine residues comprising the pair of cysteine residues. In some embodiments, the recombinant polypeptide comprises at least six disulfide bonds formed by pairs of cysteine residues comprising the pair of cysteine residues. In some embodiments, the recombinant polypeptide comprises at least seven disulfide bonds formed by pairs of cysteine residues comprising the pair of cysteine residues. In some embodiments, the recombinant polypeptide comprises at least eight disulfide bonds formed by pairs of cysteine residues comprising the pair of cysteine residues.

In some embodiments, a pair of cysteine residues that form a disulfide bond corresponds to amino acid positions: (i) Cysteine 22 and Cysteine 96 of SEQ ID NO: 1, or (ii) Cysteine 138 and Cysteine 148 of SEQ ID NO: 1, or (iii) Cysteine 199 and Cysteine 275 of SEQ ID NO: 1, or (iv) Cysteine 339 and Cysteine 407 of SEQ ID NO: 1, or (v) Cysteine 454 and Cysteine 519 of SEQ ID NO: 1, or (vi) Cysteine 565 and Cysteine 625 of SEQ ID NO: 1, or (vii) Cysteine 645 of SEQ ID NO: 1 and Cysteine 226 of SEQ ID NO: 2, or (viii) Cysteine 22 and Cysteine 96 of SEQ ID NO: 2, or (ix) Cysteine 150 and Cysteine 206 of SEQ ID NO: 2.

In some embodiments of the recombinant peptide, at least one disulfide bond formed by a pair of cysteine residues is formed by cysteine residues selected from and corresponding to positions Cysteine 22, Cysteine 96, Cysteine 138, Cysteine 148, Cysteine 199, Cysteine 275, Cysteine 339, Cysteine 407, Cysteine 454, Cysteine 519, Cysteine 565, or Cysteine 625 of SEQ ID NO: 1. In some embodiments of the recombinant polypeptide, at least one disulfide bond formed by a pair of cysteine residues is formed by cysteine residues selected from and corresponding to positions Cysteine 645 of SEQ ID NO: 1 and Cysteine 226 of SEQ ID NO: 2. In some embodiments of the recombinant peptide, at least one disulfide bond formed by a pair of cysteine residues is formed by cysteine residues selected from and corresponding to positions Cysteine 22, Cysteine 96, Cysteine 150, Cysteine 206 of SEQ ID NO: 2. The presence of disulfide bond(s) may be determined by mass spectrometry (MS).

In some embodiments, the recombinant polypeptide comprises at least one cysteine residue that is a free sulfhydryl. The presence of free sulfhydryl(s) may be determined by mass spectrometry (MS).

In some embodiments, the isolated recombinant polypeptide complex provides a maximum plasma concentration (Cmax) in a subject within about 0.001 to about 1 hour after intravenous administration, e.g., about 0.001, 0.002, 0.003, 0.004, 0.005, 0.006, 0.007, 0.008, 0.009, 0.010, 0.011, 0.012, 0.013, 0.014, 0.015, 0.016, 0.017, 0.018, 0.019, 0.020, 0.021, 0.022, 0.023, 0.024, 0.025, 0.026, 0.027, 0.028, 0.029, 0.030, 0.031, 0.032, 0.033, 0.034, 0.035, 0.036, 0.037, 0.038, 0.039, 0.040, 0.041, 0.042, 0.043, 0.044, 0.045, 0.046, 0.047, 0.048, 0.049, 0.050, 0.051, 0.052, 0.053, 0.054, 0.055, 0.056, 0.057, 0.058, 0.059, 0.060, 0.061, 0.062, 0.063, 0.064, 0.065, 0.066, 0.067, 0.068, 0.069, 0.070, 0.071, 0.072, 0.073, 0.074, 0.075, 0.076, 0.077, 0.078, 0.079, 0.080, 0.081, 0.082, 0.083, 0.084, 0.085, 0.086, 0.087, 0.088, 0.089, 0.090, 0.091, 0.092, 0.093, 0.094, 0.095, 0.096, 0.097, 0.098, 0.099, 0.100, 0.105, 0.110, 0.115, 0.120, 0.125, 0.130, 0.135, 0.140, 0.145, 0.150, 0.155, 0.160, 0.165, 0.170, 0.175, 0.180, 0.185, 0.190, 0.195, 0.200, 0.205, 0.210, 0.215, 0.220, 0.225, 0.230, 0.235, 0.240, 0.245, 0.250, 0.255, 0.260, 0.265, 0.270, 0.275, 0.280, 0.285, 0.290, 0.295, 0.300, 0.305, 0.310, 0.315, 0.320, 0.325, 0.330, 0.335, 0.340, 0.345, 0.350, 0.355, 0.360, 0.365, 0.370, 0.375, 0.380, 0.385, 0.390, 0.395, 0.400, 0.405, 0.410, 0.415, 0.420, 0.425, 0.430, 0.435, 0.440, 0.445, 0.450, 0.455, 0.460, 0.465, 0.470, 0.475, 0.480, 0.485, 0.490, 0.495, 0.500, 0.505, 0.510, 0.515, 0.520, 0.525, 0.530, 0.535, 0.540, 0.545, 0.550, 0.555, 0.560, 0.565, 0.570, 0.575, 0.580, 0.585, 0.590, 0.595, 0.600, 0.605, 0.610, 0.615, 0.620, 0.625, 0.630, 0.635, 0.640, 0.645, 0.650, 0.655, 0.660, 0.665, 0.670, 0.675, 0.680, 0.685, 0.690, 0.695, 0.700, 0.705, 0.710, 0.715, 0.720, 0.725, 0.730, 0.735, 0.740, 0.745, 0.750, 0.755, 0.760, 0.765, 0.770, 0.775, 0.780, 0.785, 0.790, 0.795, 0.800, 0.805, 0.810, 0.815, 0.820, 0.825, 0.830, 0.835, 0.840, 0.845, 0.850, 0.855, 0.860, 0.865, 0.870, 0.875, 0.880, 0.885, 0.890, 0.895, 0.900, 0.905, 0.910, 0.915, 0.920, 0.925, 0.930, 0.935, 0.940, 0.945, 0.950, 0.955, 0.960, 0.965, 0.970, 0.975, 0.980, 0.985, 0.990, 0.995, or about 1.0 hour, or any time therebetween. In some embodiments, the isolated recombinant polypeptide complex provides a Cmax in a subject within about 0.1 hour after intravenous administration.

In some embodiments, the isolated recombinant polypeptide complex provides a Cmax of about 2000 to 4000 ng/ml in a subject within about 0.1 hour after intravenous administration of a dose of about 0.1 mg/kg, e.g., about 2000 to 4000 ng/ml, 2000 to 3500 ng/ml, 2000 to 3000 ng/ml, 2000 to 2500 ng/ml, 2500 to 4000 ng/ml, 2500 to 3500 ng/ml, 2500 to 3000 ng/ml, 3000 to 4000 ng/ml, 3000 to 3500 ng/ml, or about 3500 to 4000 ng/ml, or any concentration therebetween. In some embodiments, the isolated recombinant polypeptide complex provides a Cmax of about 2500 to 3500 ng/ml in a subject within about 0.1 hour after intravenous administration of a dose of about 0.1 mg/kg.

In some embodiments, the isolated recombinant polypeptide complex provides a Cmax of about 7000 to 12000 ng/ml in a subject within about 0.1 hour after intravenous administration of a dose of about 0.3 mg/kg, e.g., about 7000 to 12000 ng/ml, 7000 to 11500 ng/ml, 7000 to 11000 ng/ml, 7000 to 10500 ng/ml, 7000 to 10000 ng/ml, 7000 to 9500 ng/ml, 7000 to 9000 ng/ml, 7000 to 8500 ng/ml, 7000 to 8000 ng/ml, 7000 to 7500 ng/ml, 7500 to 12000 ng/ml, 7500 to 11500 ng/ml, 7500 to 11000 ng/ml, 7500 to 10500 ng/ml, 7500 to 10000 ng/ml, 7500 to 9500 ng/ml, 7500 to 9000 ng/ml, 7500 to 8500 ng/ml, 7500 to 8000 ng/ml, 8000 to 12000 ng/ml, 8000 to 11500 ng/ml, 8000 to 11000 ng/ml, 8000 to 10500 ng/ml, 8000 to 10000 ng/ml, 8000 to 9500 ng/ml, 8000 to 9000 ng/ml, 8000 to 8500 ng/ml, 8500 to 12000 ng/ml, 8500 to 11500 ng/ml, 8500 to 11000 ng/ml, 8500 to 10500 ng/ml, 8500 to 10000 ng/ml, 8500 to 9500 ng/ml, 8500 to 9000 ng/ml, 9000 to 12000 ng/ml, 9000 to 11500 ng/ml, 9000 to 11000 ng/ml, 9000 to 10500 ng/ml, 9000 to 10000 ng/ml, 9000 to 9500 ng/ml, 9500 to 12000 ng/ml, 9500 to 11500 ng/ml, 9500 to 11000 ng/ml, 9500 to 10500 ng/ml, 9500 to 10000 ng/ml, 10000 to 12000 ng/ml, 10000 to 11500 ng/ml, 10000 to 11000 ng/ml, 10000 to 10500 ng/ml, 10500 to 12000 ng/ml, 10500 to 11500 ng/ml, 10500 to 11000 ng/ml, 11000 to 12000 ng/ml, 11000 to 11500 ng/ml, or about 11500 to 12000 ng/ml, or any range therebetween. In some embodiments, the isolated recombinant polypeptide complex provides a Cmax of about 7500 to 10500 ng/ml in a subject within about 0.1 hour after intravenous administration of a dose of about 0.3 mg/kg.

In some embodiments, the isolated recombinant polypeptide complex provides a Cmax of about 32500 to 57500 ng/ml in a subject within about 0.1 hour after intravenous administration of a dose of about 1.5 mg/kg, e.g., about 32500 to 57500 ng/ml, 32500 to 55000 ng/ml, 32500 to 52500 ng/ml, 32500 to 50000 ng/ml, 32500 to 47500 ng/ml, 32500 to 45000 ng/ml, 32500 to 42500 ng/ml, 32500 to 40000 ng/ml, 32500 to 37500 ng/ml, 32500 to 35000 ng/ml, 35000 to 57500 ng/ml, 35000 to 55000 ng/ml, 35000 to 52500 ng/ml, 35000 to 50000 ng/ml, 35000 to 47500 ng/ml, 35000 to 45000 ng/ml, 35000 to 42500 ng/ml, 35000 to 40000 ng/ml, 35000 to 37500 ng/ml, 37500 to 75000 ng/ml, 37500 to 72500 ng/ml, 37500 to 70000 ng/ml, 37500 to 67500 ng/ml, 37500 to 65000 ng/ml, 37500 to 62500 ng/ml, 37500 to 60000 ng/ml, 37500 to 57500 ng/ml, 37500 to 55000 ng/ml, 37500 to 52500 ng/ml, 37500 to 50000 ng/ml, 37500 to 47500 ng/ml, 37500 to 45000 ng/ml, 37500 to 42500 ng/ml, 37500 to 40000 ng/ml, 40000 to 57500 ng/ml, 40000 to 55000 ng/ml, 40000 to 52500 ng/ml, 40000 to 50000 ng/ml, 40000 to 47500 ng/ml, 40000 to 45000 ng/ml, 40000 to 42500 ng/ml, 42500 to 57500 ng/ml, 42500 to 55000 ng/ml, 42500 to 52500 ng/ml, 42500 to 50000 ng/ml, 42500 to 47500 ng/ml, 42500 to 45000 ng/ml, 45000 to 57500 ng/ml, 45000 to 55000 ng/ml, 45000 to 52500 ng/ml, 45000 to 50000 ng/ml, 45000 to 47500 ng/ml, 47500 to 57500 ng/ml, 47500 to 55000 ng/ml, 47500 to 52500 ng/ml, 47500 to 50000 ng/ml, 50000 to 57500 ng/ml, 50000 to 55000 ng/ml, 50000 to 52500 ng/ml, 52500 to 57500 ng/ml, 52500 to 55000 ng/ml, or about 55000 to 57500 ng/ml, or concentration range therebetween. In some embodiments, the isolated recombinant polypeptide complex provides a Cmax of about 37500 to 52500 ng/ml in a subject within about 0.1 hour after intravenous administration of a dose of about 1.5 mg/kg.

In some embodiments, the isolated recombinant polypeptide complex provides a half maximum plasma concentration in a subject in about 50 to about 130 hours (T_{1/2}) after intravenous administration, e.g., about 50 to about 130 hours, about 50 to about 120 hours, about 50 to about 110 hours, about 50 to about 100 hours, about 50 to about 90 hours, about 50 to about 80 hours, about 50 to about 70 hours, about 50 to about 60 hours, about 60 to about 130 hours, about 60 to about 120 hours, about 60 to about 110 hours, about 60 to about 100 hours, about 60 to about 90 hours, about 60 to about 80 hours, about 60 to about 70 hours, 70 to about 130 hours, about 70 to about 120 hours, about 70 to about 110 hours, about 70 to about 100 hours, about 70 to about 90 hours, about 70 to about 80 hours, about 80 to about 130 hours, about 80 to about 120 hours, about 80 to about 110 hours, about 80 to about 100 hours, about 80 to about 90 hours, about 90 to about 130 hours, about 90 to about 120 hours, about 90 to about 110 hours, about 90 to about 100 hours, about 100 to about 130 hours, about 100 to about 120 hours, about 100 to about 110 hours, about 110 to about 130 hours, about 110 to about 120 hours, or about 120 to about 130 hours, or any duration therebetween.

In some embodiments, the value of Cmax correlates with the dose of the isolated recombinant polypeptide complex that is administered. In some embodiments, the value of Cmax is proportional to the dose of the isolated recombinant polypeptide complex that is administered.

In some embodiments, less than about 10% of the isolated recombinant polypeptide complex degrades each day in the serum of a subject, e.g., less than about 10%, 9.9%, 9.8%, 9.7%, 9.6%, 9.5%, 9.4%, 9.3%, 9.2%, 9.1%, 9%, 8.9%, 8.8%, 8.7%, 8.6%, 8.5%, 8.4%, 8.3%, 8.2%, 8.1%, 8%, 7.9%, 7.8%, 7.7%, 7.6%, 7.5%, 7.4%, 7.3%, 7.2%, 7.1%, 7%, 6.9%, 6.8%, 6.7%, 6.6%, 6.5%, 6.4%, 6.3%, 6.2%, 6.1%, 6%, 5.9%, 5.8%, 5.7%, 5.6%, 5.5%, 5.4%, 5.3%, 5.2%, 5.1%, 5%, 4.9%, 4.8%, 4.7%, 4.6%, 4.5%, 4.4%, 4.3%, 4.2%, 4.1%, 4%, 3.9%, 3.8%, 3.7%, 3.6%, 3.5%, 3.4%, 3.3%, 3.2%, 3.1%, 3%, 2.9%, 2.8%, 2.7%, 2.6%, 2.5%, 2.4%, 2.3%, 2.2%, 2.1%, 2%, 1.9%, 1.8%, 1.7%, 1.6%, 1.5%, 1.4%, 1.3%, 1.2%, 1.1%, 1%, 0.9%, 0.8%, 0.7%, 0.6%, 0.5%, 0.4%, 0.3%, 0.2%, or less than about 0.1%, or any percentage therebetween. In some embodiments, less than 1% of the isolated recombinant polypeptide complex degrades each day in the serum of a subject.

In some embodiments, the subject is diagnosed with cancer. In some embodiments, the cancer comprises prostate cancer. In some embodiments, the cancer comprises metastatic castration-resistant prostate cancer (mCRPC).

In some embodiments, the isolated recombinant polypeptide complex is cleaved by a tumor specific protease to generate an enzymatic product of the isolated recombinant polypeptide complex after the administering. In some embodiments, the tumor specific protease comprises two or more proteases. In some embodiments, the isolated recombinant polypeptide complex is cleaved by a first protease of the two or more proteases to generate a first metabolic product of the isolated recombinant polypeptide complex. In some embodiments, the isolated recombinant polypeptide complex is cleaved by a second protease of the two or more proteases to generate a second metabolic product of the isolated recombinant polypeptide complex. In some embodiments, the first protease comprises a serine protease. In some embodiments, the second protease comprises a matrix metalloprotease. In some embodiments, the serine protease comprises recombinant human matriptase (MTSP1). In some embodiments, the matrix metalloprotease comprises recombinant human matrix metalloprotease 9 (MMP9).

In some embodiments, the first metabolic product of the isolated recombinant polypeptide complex comprises an amino acid sequence with at least 80% sequence identity to SEQ ID NO: 5. In some embodiments, the first metabolic product of the isolated recombinant polypeptide complex comprises an amino acid sequence with at least 81% sequence identity to SEQ ID NO: 5. In some embodiments, the first metabolic product of the isolated recombinant polypeptide complex comprises an amino acid sequence with at least 82% sequence identity to SEQ ID NO: 5. In some embodiments, the first metabolic product of the isolated recombinant polypeptide complex comprises an amino acid sequence with at least 83% sequence identity to SEQ ID NO: 5. In some embodiments, the first metabolic product of the isolated recombinant polypeptide complex comprises an amino acid sequence with at least 84% sequence identity to SEQ ID NO: 5. In some embodiments, the first metabolic product of the isolated recombinant polypeptide complex comprises an amino acid sequence with at least 85% sequence identity to SEQ ID NO: 5. In some embodiments, the first metabolic product of the isolated recombinant polypeptide complex comprises an amino acid sequence with at least 86% sequence identity to SEQ ID NO: 5. In some embodiments, the first metabolic product of the isolated recombinant polypeptide complex comprises an amino acid sequence with at least 87% sequence identity to SEQ ID NO: 5. In some embodiments, the first metabolic product of the isolated recombinant polypeptide complex comprises an amino acid sequence with at least 88% sequence identity to SEQ ID NO: 5. In some embodiments, the first metabolic product of the isolated recombinant polypeptide complex comprises an amino acid sequence with at least 89% sequence identity to SEQ ID NO: 5. In some embodiments, the first metabolic product of the isolated recombinant polypeptide complex comprises an amino acid sequence with at least 90% sequence identity to SEQ ID NO: 5. In some embodiments, the first metabolic product of the isolated recombinant polypeptide complex comprises an amino acid sequence with at least 91% sequence identity to SEQ ID NO: 5. In some embodiments, the first metabolic product of the isolated recombinant polypeptide complex comprises an amino acid sequence with at least 92% sequence identity to SEQ ID NO: 5. In some embodiments, the first metabolic product of the isolated recombinant polypeptide complex comprises an amino acid sequence with at least 93% sequence identity to SEQ ID NO: 5. In some embodiments, the first metabolic product of the isolated recombinant polypeptide complex comprises an amino acid sequence with at least 94% sequence identity to SEQ ID NO: 5. In some embodiments, the first metabolic product of the isolated recombinant polypeptide complex comprises an amino acid sequence with at least 95% sequence identity to SEQ ID NO: 5. In some embodiments, the first metabolic product of the isolated recombinant polypeptide complex comprises an amino acid sequence with at least 96% sequence identity to SEQ ID NO: 5. In some embodiments, the first metabolic product of the isolated recombinant polypeptide complex comprises an amino acid sequence with at least 97% sequence identity to SEQ ID NO: 5. In some embodiments, the first metabolic product of the isolated recombinant polypeptide complex comprises an amino acid sequence with at least 98% sequence identity to SEQ ID NO: 5. In some embodiments, the first metabolic product of the isolated recombinant polypeptide complex comprises an amino acid sequence with at least 99% sequence identity to SEQ ID NO: 5. In some embodiments, the first metabolic product of the isolated recombinant polypeptide complex comprises the amino acid sequence of SEQ ID NO: 5.

In some embodiments, the second metabolic product of the isolated recombinant polypeptide complex comprises an amino acid sequence with at least 80% sequence identity to SEQ ID NO: 6. In some embodiments, the second metabolic product of the isolated recombinant polypeptide complex comprises an amino acid sequence with at least 81% sequence identity to SEQ ID NO: 6. In some embodiments, the second metabolic product of the isolated recombinant polypeptide complex comprises an amino acid sequence with at least 82% sequence identity to SEQ ID NO: 6. In some embodiments, the second metabolic product of the isolated recombinant polypeptide complex comprises an amino acid sequence with at least 83% sequence identity to SEQ ID NO: 6. In some embodiments, the second metabolic product of the isolated recombinant polypeptide complex comprises an amino acid sequence with at least 84% sequence identity to SEQ ID NO: 6. In some embodiments, the second metabolic product of the isolated recombinant polypeptide complex comprises an amino acid sequence with at least 85% sequence identity to SEQ ID NO: 6. In some embodiments, the second metabolic product of the isolated recombinant polypeptide complex comprises an amino acid sequence with at least 86% sequence identity to SEQ ID NO: 6. In some embodiments, the second metabolic product of the isolated recombinant polypeptide complex comprises an amino acid sequence with at least 87% sequence identity to SEQ ID NO: 6. In some embodiments, the second metabolic product of the isolated recombinant polypeptide complex comprises an amino acid sequence with at least 88% sequence identity to SEQ ID NO: 6. In some embodiments, the second metabolic product of the isolated recombinant polypeptide complex comprises an amino acid sequence with at least 89% sequence identity to SEQ ID NO: 6. In some embodiments, the second metabolic product of the isolated recombinant polypeptide complex comprises an amino acid sequence with at least 90% sequence identity to SEQ ID NO: 6. In some embodiments, the second metabolic product of the isolated recombinant polypeptide complex comprises an amino acid sequence with at least 91% sequence identity to SEQ ID NO: 6. In some embodiments, the second metabolic product of the isolated recombinant polypeptide complex comprises an amino acid sequence with at least 92% sequence identity to SEQ ID NO: 6. In some embodiments, the second metabolic product of the isolated recombinant polypeptide complex comprises an amino acid sequence with at least 93% sequence identity to SEQ ID NO: 6. In some embodiments, the second metabolic product of the isolated recombinant polypeptide complex comprises an amino acid sequence with at least 94% sequence identity to SEQ ID NO: 6. In some embodiments, the second metabolic product of the isolated recombinant polypeptide complex comprises an amino acid sequence with at least 95% sequence identity to SEQ ID NO: 6. In some embodiments, the second metabolic product of the isolated recombinant polypeptide complex comprises an amino acid sequence with at least 96% sequence identity to SEQ ID NO: 6. In some embodiments, the second metabolic product of the isolated recombinant polypeptide complex comprises an amino acid sequence with at least 97% sequence identity to SEQ ID NO: 6. In some embodiments, the second metabolic product of the isolated recombinant polypeptide complex comprises an amino acid sequence with at least 98% sequence identity to SEQ ID NO: 6. In some embodiments, the second metabolic product of the isolated recombinant polypeptide complex comprises an amino acid sequence with at least 99% sequence identity to SEQ ID NO: 6. In some embodiments, the second metabolic product of the isolated recombinant polypeptide complex comprises the amino acid sequence of SEQ ID NO: 6.

In some embodiments, the subject is a non-human primate or human. In some embodiments, the non-human primate is cynomolgus monkey.

Disclosed herein, in another aspect, is an isolated polypeptide comprising an amino acid sequence with at least 80% sequence identity to SEQ ID NO: 5 and the isolated polypeptide is 484 amino acids in length.

Disclosed herein, in another aspect, is an isolated polypeptide comprising an amino acid sequence with at least 81% sequence identity to SEQ ID NO: 5 and the isolated polypeptide is 484 amino acids in length.

Disclosed herein, in another aspect, is an isolated polypeptide comprising an amino acid sequence with at least 82% sequence identity to SEQ ID NO: 5 and the isolated polypeptide is 484 amino acids in length.

Disclosed herein, in another aspect, is an isolated polypeptide comprising an amino acid sequence with at least 83% sequence identity to SEQ ID NO: 5 and the isolated polypeptide is 484 amino acids in length.

Disclosed herein, in another aspect, is an isolated polypeptide comprising an amino acid sequence with at least 84% sequence identity to SEQ ID NO: 5 and the isolated polypeptide is 484 amino acids in length.

Disclosed herein, in another aspect, is an isolated polypeptide comprising an amino acid sequence with at least 85% sequence identity to SEQ ID NO: 5 and the isolated polypeptide is 484 amino acids in length.

Disclosed herein, in another aspect, is an isolated polypeptide comprising an amino acid sequence with at least 86% sequence identity to SEQ ID NO: 5 and the isolated polypeptide is 484 amino acids in length.

Disclosed herein, in another aspect, is an isolated polypeptide comprising an amino acid sequence with at least 87% sequence identity to SEQ ID NO: 5 and the isolated polypeptide is 484 amino acids in length.

Disclosed herein, in another aspect, is an isolated polypeptide comprising an amino acid sequence with at least 88% sequence identity to SEQ ID NO: 5 and the isolated polypeptide is 484 amino acids in length.

Disclosed herein, in another aspect, is an isolated polypeptide comprising an amino acid sequence with at least 89% sequence identity to SEQ ID NO: 5 and the isolated polypeptide is 484 amino acids in length.

Disclosed herein, in another aspect, is an isolated polypeptide comprising an amino acid sequence with at least 90% sequence identity to SEQ ID NO: 5 and the isolated polypeptide is 484 amino acids in length.

Disclosed herein, in another aspect, is an isolated polypeptide comprising an amino acid sequence with at least 91% sequence identity to SEQ ID NO: 5 and the isolated polypeptide is 484 amino acids in length.

Disclosed herein, in another aspect, is an isolated polypeptide comprising an amino acid sequence with at least 92% sequence identity to SEQ ID NO: 5 and the isolated polypeptide is 484 amino acids in length.

Disclosed herein, in another aspect, is an isolated polypeptide comprising an amino acid sequence with at least 93% sequence identity to SEQ ID NO: 5 and the isolated polypeptide is 484 amino acids in length.

Disclosed herein, in another aspect, is an isolated polypeptide comprising an amino acid sequence with at least 94% sequence identity to SEQ ID NO: 5 and the isolated polypeptide is 484 amino acids in length.

Disclosed herein, in another aspect, is an isolated polypeptide comprising an amino acid sequence with at least 95% sequence identity to SEQ ID NO: 5 and the isolated polypeptide is 484 amino acids in length.

Disclosed herein, in another aspect, is an isolated polypeptide comprising an amino acid sequence with at least 96% sequence identity to SEQ ID NO: 5 and the isolated polypeptide is 484 amino acids in length.

Disclosed herein, in another aspect, is an isolated polypeptide comprising an amino acid sequence with at least 97% sequence identity to SEQ ID NO: 5 and the isolated polypeptide is 484 amino acids in length.

Disclosed herein, in another aspect, is an isolated polypeptide comprising an amino acid sequence with at least 98% sequence identity to SEQ ID NO: 5 and the isolated polypeptide is 484 amino acids in length.

Disclosed herein, in another aspect, is an isolated polypeptide comprising an amino acid sequence with at least 99% sequence identity to SEQ ID NO: 5 and the isolated polypeptide is 484 amino acids in length.

Disclosed herein, in another aspect, is an isolated polypeptide comprising the amino acid sequence of SEQ ID NO: 5 and the isolated polypeptide is 484 amino acids in length.

Disclosed herein, in another aspect, is an isolated polypeptide comprising an amino acid sequence with at least 80% sequence identity to SEQ ID NO: 6 and the isolated polypeptide is 476 amino acids in length.

Disclosed herein, in another aspect, is an isolated polypeptide comprising an amino acid sequence with at least 81% sequence identity to SEQ ID NO: 6 and the isolated polypeptide is 476 amino acids in length.

Disclosed herein, in another aspect, is an isolated polypeptide comprising an amino acid sequence with at least 82% sequence identity to SEQ ID NO: 6 and the isolated polypeptide is 476 amino acids in length.

Disclosed herein, in another aspect, is an isolated polypeptide comprising an amino acid sequence with at least 83% sequence identity to SEQ ID NO: 6 and the isolated polypeptide is 476 amino acids in length.

Disclosed herein, in another aspect, is an isolated polypeptide comprising an amino acid sequence with at least 84% sequence identity to SEQ ID NO: 6 and the isolated polypeptide is 476 amino acids in length.

Disclosed herein, in another aspect, is an isolated polypeptide comprising an amino acid sequence with at least 85% sequence identity to SEQ ID NO: 6 and the isolated polypeptide is 476 amino acids in length.

Disclosed herein, in another aspect, is an isolated polypeptide comprising an amino acid sequence with at least 86% sequence identity to SEQ ID NO: 6 and the isolated polypeptide is 476 amino acids in length.

Disclosed herein, in another aspect, is an isolated polypeptide comprising an amino acid sequence with at least 87% sequence identity to SEQ ID NO: 6 and the isolated polypeptide is 476 amino acids in length.

Disclosed herein, in another aspect, is an isolated polypeptide comprising an amino acid sequence with at least 88% sequence identity to SEQ ID NO: 6 and the isolated polypeptide is 476 amino acids in length.

Disclosed herein, in another aspect, is an isolated polypeptide comprising an amino acid sequence with at least 89% sequence identity to SEQ ID NO: 6 and the isolated polypeptide is 476 amino acids in length.

Disclosed herein, in another aspect, is an isolated polypeptide comprising an amino acid sequence with at least 90% sequence identity to SEQ ID NO: 6 and the isolated polypeptide is 476 amino acids in length.

Disclosed herein, in another aspect, is an isolated polypeptide comprising an amino acid sequence with at least 91% sequence identity to SEQ ID NO: 6 and the isolated polypeptide is 476 amino acids in length.

Disclosed herein, in another aspect, is an isolated polypeptide comprising an amino acid sequence with at least 92% sequence identity to SEQ ID NO: 6 and the isolated polypeptide is 476 amino acids in length.

Disclosed herein, in another aspect, is an isolated polypeptide comprising an amino acid sequence with at least 93% sequence identity to SEQ ID NO: 6 and the isolated polypeptide is 476 amino acids in length.

Disclosed herein, in another aspect, is an isolated polypeptide comprising an amino acid sequence with at least 94% sequence identity to SEQ ID NO: 6 and the isolated polypeptide is 476 amino acids in length.

Disclosed herein, in another aspect, is an isolated polypeptide comprising an amino acid sequence with at least 95% sequence identity to SEQ ID NO: 6 and the isolated polypeptide is 476 amino acids in length.

Disclosed herein, in another aspect, is an isolated polypeptide comprising an amino acid sequence with at least 96% sequence identity to SEQ ID NO: 6 and the isolated polypeptide is 476 amino acids in length.

Disclosed herein, in another aspect, is an isolated polypeptide comprising an amino acid sequence with at least 97% sequence identity to SEQ ID NO: 6 and the isolated polypeptide is 476 amino acids in length.

Disclosed herein, in another aspect, is an isolated polypeptide comprising an amino acid sequence with at least 98% sequence identity to SEQ ID NO: 6 and the isolated polypeptide is 476 amino acids in length.

Disclosed herein, in another aspect, is an isolated polypeptide comprising an amino acid sequence with at least 99% sequence identity to SEQ ID NO: 6 and the isolated polypeptide is 476 amino acids in length.

Disclosed herein, in another aspect, is an isolated polypeptide comprising the amino acid sequence of SEQ ID NO: 6 and the isolated polypeptide is 476 amino acids in length.

### Amino Acid Modifications

In some embodiments, at least one glutamine of SEQ ID NO: 1 or SEQ ID NO:2 is cyclized to form a pyroglutamine. In some embodiments, the pyroglutamine is on the N-terminal of SEQ ID NO: 2. In some embodiments, the recombinant polypeptide comprises a pyroglutamine residue at an amino acid position corresponding to Glutamine 1 of SEQ ID NO: 2. The presence and amount of pyroglutamine residue(s) may be determined by mass spectrometry (MS).

### Thermal Stability

In some embodiments, the recombinant polypeptide is characterized by a melting temperature (Tₘ) of about 70° C., 71° C., 72° C., 73° C., 74° C., 75° C., 76° C., 77° C., 78° C., 79° C., or 80° C., or a range between any two of the foregoing values. In some embodiments, the recombinant polypeptide is characterized by a melting temperature (Tₘ) from about 70° C. to about 81° C. In some embodiments, the recombinant polypeptide is characterized by a melting temperature (Tₘ) from about 71° C. to about 72° C. In some embodiments, the recombinant polypeptide is characterized by a melting temperature (Tₘ) from about 78° C. to about 80° C. In some embodiments, the recombinant polypeptide is characterized by a melting temperature (Tₘ) from about 71.4° C. In some embodiments, the recombinant polypeptide is characterized by a melting temperature (Tₘ) from about 79.5° C. In some embodiments, the recombinant polypeptide is characterized by a melting onset temperature (Tₒₙₛₑₜ) from about 55° C. to about 60° C. In some embodiments, the recombinant polypeptide is characterized by a melting onset temperature (Tₒₙₛₑₜ) from about 58° C. to about 60° C. In some embodiments, the recombinant polypeptide is characterized by a melting onset temperature (Tₒₙₛₑₜ) from about 59° C. The melting temperature (Tₘ) may be determined by Differential Scanning Calorimetry (DSC). The melting onset temperature (Tₒₙₛₑₜ) may be determined by Differential Scanning Calorimetry (DSC).

### Structure Composition

In some embodiments the recombinant polypeptide has a secondary structure composition comprising a β-sheet or random coil. The secondary structure composition can comprise a β-sheet. The secondary structure composition can comprise a random coil.

In some embodiments, the recombinant polypeptide is characterized by a near UV circular dichroism peak at a wavelength less than or equal to 300 nm, 295 nm, 290 nm, 285 nm, 280 nm, or 275 nm. In some embodiments, the recombinant polypeptide is characterized by a near UV circular dichroism peak at a wavelength greater than or equal to 275 nm, 280 nm, 285 nm, 290 nm, or 300 nm. In some embodiments, the recombinant polypeptide is characterized by a near UV circular dichroism peak at a wavelength between 275 nm and 285 nm, between 280 nm and 290 nm, between 285 nm and 295 nm, or between 290 nm and 300 nm.

In some embodiments, the recombinant polypeptide is characterized by a far UV circular dichroism peak at a wavelength less than or equal to 220 nm, 210 nm, or 205 nm. In some embodiments, the recombinant polypeptide is characterized by a far UV circular dichroism peak at a wavelength greater than or equal to 205 nm, 210 nm, or 220 nm. In some embodiments, the recombinant polypeptide is characterized by a far UV circular dichroism peak at a wavelength between 200 nm and 210 nm or between 205 nm and 220 nm.

### One or More Characteristics

A polypeptide or recombinant polypeptide described herein may have one or more characteristics as described hereinabove in this section.

In some embodiments, the recombinant polypeptide has at least one, at least two, at least three, at least four, at least five, at least six, or all seven of the characteristics (a)-(g):
(a) at least one disulfide bond formed by a pair of cysteine residues in the first chain or the second chain or by a pair of cysteine residues in the first chain and the second chain; or
(b) a secondary structure composition comprising a β-sheet or a random coil;
(c) at least one pyroglutamine in the second chain;
(d) a melting temperature (Tm) between of about 65° C. to about 85° C. when the isolated recombinant polypeptide complex is formulated at a concentration of 1.0 mg/mL in 10 mM His, 8 (w/v) % sucrose, 0.01 (w/v) % polysorbate-20, pH 6.3;
(e) a far UV circular dichroism peak at a wavelength between 190 nm and 205 nm when the isolated recombinant polypeptide complex is formulated at a concentration of 0.1 mg/mL in 10 mM potassium phosphate buffer pH 7.0;
(f) a far UV circular dichroism dip at a wavelength between 210 nm and 220 nm when the isolated recombinant polypeptide complex is formulated at a concentration of 0.1 mg/mL in 10 mM potassium phosphate buffer pH 7.0; or
(g) a near UV circular dichroism peak at a wavelength between 250 nm and 300 nm when the isolated recombinant polypeptide complex is formulated at a concentration of 1.0 mg/mL in 10 mM His, 8% (w/v) sucrose, 0.01% (w/v) polysorbate-20, pH 6.3

In some embodiments, the recombinant polypeptide comprises at least one (e.g., one, two, three, four, five, six, or seven) of the characteristics (a)-(g). In some embodiments, the recombinant polypeptide comprises at least two (e.g., two, three, four, five, six, or seven) of the characteristics (a)-(g). In some embodiments, the recombinant polypeptide comprises at least three (e.g., three, four, five, six, or seven) of the characteristics (a)-(g). In some embodiments, the recombinant polypeptide comprises at least four (e.g., four, five, six or seven) of the characteristics (a)-(g). In some embodiments, the recombinant polypeptide comprises at least five (e.g., five, six or seven) of the characteristics (a)-(g). In some embodiments, the recombinant polypeptide comprises at least six (e.g., six or seven) of the characteristics (a)-(g). In some embodiments, the recombinant polypeptide comprises all seven of the characteristics (a)-(g).

### Formulations

Disclosed herein includes formulation(s) comprising a population of polypeptides or recombinant polypeptides, such as comprising any one or a combination the polypeptide(s) or recombinant polypeptide(s) as described herein.

In some embodiments of the formulation, at least about 80%, 85%, 90%, or 95% (e.g., by mole or by mass) of the polypeptides of the population comprise at least one pyroglutamine residue (such as any described hereinabove). In some embodiments, at least about 80% (e.g., by mole or by mass) of the polypeptides of the population comprise at least one pyroglutamine residue, e.g., at an amino acid position corresponding to Glutamine 1 of SEQ ID NO: 2. In some embodiments, at least about 85% (e.g., by mole or by mass) of the polypeptides of the population comprise at least one pyroglutamine residue, e.g., at an amino acid position corresponding to Glutamine 1 of SEQ ID NO: 2. In some embodiments, at least about 90% (e.g., by mole or by mass) of the polypeptides of the population comprise at least one pyroglutamine residue, e.g., at an amino acid position corresponding to Glutamine 1 of SEQ ID NO: 2. In some embodiments, at least about 95% (e.g., by mole or by mass) of the polypeptides of the population comprise at least one pyroglutamine residue, e.g., at an amino acid position corresponding to Glutamine 1 of SEQ ID NO: 2.

In some embodiments of the formulation, at least 90%, 91%, 92%, 93%, 94%, 95%, 96%, 97%, 98%, or 99% (e.g., by mole or by mass) of the polypeptides of the population is monomeric. In some embodiments of the formulation, less than or equal to 5%, 4%, 3%, 2%, 1%, 0.5%, or 0.1% (e.g., by mole or by mass) of the polypeptides of the population is aggregated.

In some embodiments of the formulation, the polypeptides of the population comprises at least about 85%, 86%, 87%, 88%, 89%, 90%, 91%, 92%, 93%, 94%, 95%, 96%, 97%, 98%, or 99% sequence identity to SEQ ID NO: 1. In some embodiments, the polypeptides of the population comprises at least about 85% sequence identity to SEQ ID NO: 1. In some embodiments, the polypeptides of the population comprises at least about 90% sequence identity to SEQ ID NO: 1. In some embodiments, the polypeptides of the population comprises at least about 95% sequence identity to SEQ ID NO: 1. In some embodiments, the polypeptides of the population comprises at least about 99% sequence identity to SEQ ID NO: 1. In some embodiments, the polypeptides of the population comprises an amino acid sequence according to SEQ ID NO: 1.

In some embodiments of the formulation, the polypeptides of the population comprises at least about 85%, 86%, 87%, 88%, 89%, 90%, 91%, 92%, 93%, 94%, 95%, 96%, 97%, 98%, or 99% sequence identity to SEQ ID NO: 2. In some embodiments, the polypeptides of the population comprises at least about 85% sequence identity to SEQ ID NO: 2. In some embodiments, the polypeptides of the population comprises at least about 90% sequence identity to SEQ ID NO: 2. In some embodiments, the polypeptides of the population comprises at least about 95% sequence identity to SEQ ID NO: 2. In some embodiments, the polypeptides of the population comprises at least about 99% sequence identity to SEQ ID NO: 2. In some embodiments, the polypeptides of the population comprises an amino acid sequence according to SEQ ID NO: 2.

Disclosed herein is a pharmaceutical composition comprising one or more pharmaceutically acceptable excipients, wherein the one or more pharmaceutically acceptable excipients comprise histidine, sucrose, polysorbate-20, sodium phosphate, citrate, glutamate, glycine, arginine, sorbitol, arginine hydrochloride, acetate, sodium chloride, potassium chloride, magnesium chloride, and calcium chloride. Disclosed herein is a pharmaceutical composition, wherein the pharmaceutical composition comprises the recombinant polypeptide (such as any described herein), sodium phosphate monobasic monohydrate, sodium phosphate dibasic, heptahydrate, sodium chloride, potassium chloride, histidine, citrate, acetate, sucrose, polysorbate-20, polysorbate-80, magnesium chloride hexahydrate and calcium chloride dihydrate. The pharmaceutical composition can have a pH less than or equal to 7.0, 6.5, 6.0, or 5.5. The pharmaceutical composition can have a pH greater than or equal to 5.5, 6.0, 6.5, or 7.0. The pharmaceutical composition can comprise greater than or equal to 5 mM, 6 mM, 7 mM, 8 mM, 9 mM, or 10 mM Histidine. The pharmaceutical composition can comprise less than or equal to 10 mM, 9 mM, 8 mM, 7 mM, 6 mM, or 5 mM Histidine. The pharmaceutical composition can comprise greater than or equal to 5% (w/v), 6% (w/v), 7% (w/v), or 8% (w/v) sucrose. The pharmaceutical composition can comprise less than or equal to 9% (w/v), 8% (w/v), 7% (w/v), 6% (w/v), or 5% (w/v) sucrose. The pharmaceutical composition can comprise less than or equal to 0.05% (w/v) polysorbate 20, 0.04% (w/v) polysorbate 20, 0.03% (w/v) polysorbate 20, 0.02% (w/v) polysorbate 20, or 0.01% polysorbate 20. The pharmaceutical composition can comprise greater than or equal to 0.01% (w/v) polysorbate 20, 0.02% (w/v) polysorbate 20, 0.03% (w/v) polysorbate 20, 0.04% (w/v) polysorbate 20, or 0.05% polysorbate 20. Disclosed herein is a pharmaceutical composition, wherein the pharmaceutical composition comprises the recombinant polypeptide (such as any described herein), 10 mM Histidine, 8% (w/v) sucrose, and 0.01% (w/v) polysorbate-20.

Disclosed herein, in another aspect, is a pharmaceutical composition. In some embodiments, the pharmaceutical composition comprises: (a) an isolated recombinant polypeptide complex disclosed herein; and (b) a pharmaceutically acceptable excipient.

In some embodiments, the pharmaceutically acceptable excipient comprises a buffer, a stabilizing agent, a tonicity agent, a surfactant, or combinations thereof. In some embodiments, the buffer comprises an amino acid or a derivative thereof. In some embodiments, the amino acid or the derivative thereof comprises L-histidine, L-histidine monohydrochloride monohydrate, or combinations thereof. In some embodiments, the stabilizing agent comprises sugar. In some embodiments, the sugar comprises sucrose. In some embodiments, the tonicity agent comprises sugar. In some embodiments, the sugar comprises sucrose. In some embodiments, the surfactant comprises a polysorbate. In some embodiments, the surfactant comprises polysorbate 20, polysorbate 40, polysorbate 60, or polysorbate 80. In some embodiments, the surfactant comprises polysorbate 20. In some embodiments, the total amount of L-histidine in the pharmaceutical composition is about 1 mM, 1.1 mM, 1.2 mM, 1.3 mM, 1.4 mM, 1.5 mM, 1.6 mM, 1.7 mM, 1.8 mM, 1.9 mM, 2 mM, 2.1 mM, 2.2 mM, 2.3 mM, 2.4 mM, 2.5 mM, 2.6 mM, 2.7 mM, 2.8 mM, 2.9 mM, 3 mM, 3.1 mM, 3.2 mM, 3.3 mM, 3.4 mM, 3.5 mM, 3.6 mM, 3.7 mM, 3.8 mM, 3.9 mM, 4 mM, 4.1 mM, 4.2 mM, 4.3 mM, 4.4 mM, 4.5 mM, 4.6 mM, 4.7 mM, 4.8 mM, 4.9 mM, 5 mM, 5.1 mM, 5.2 mM, 5.3 mM, 5.4 mM, 5.5 mM, 5.6 mM, 5.7 mM, 5.8 mM, 5.9 mM, 6 mM, 6.1 mM, 6.2 mM, 6.3 mM, 6.4 mM, 6.5 mM, 6.6 mM, 6.7 mM, 6.8 mM, 6.9 mM, 7 mM, 7.1 mM, 7.2 mM, 7.3 mM, 7.4 mM, 7.5 mM, 7.6 mM, 7.7 mM, 7.8 mM, 7.9 mM, 8 mM, 8.1 mM, 8.2 mM, 8.3 mM, 8.4 mM, 8.5 mM, 8.6 mM, 8.7 mM, 8.8 mM, 8.9 mM, 9 mM, 9.1 mM, 9.2 mM, 9.3 mM, 9.4 mM, 9.5 mM, 9.6 mM, 9.7 mM, 9.8 mM, 9.9 mM, 10 mM, 10.1 mM, 10.2 mM, 10.3 mM, 10.4 mM, 10.5 mM, 10.6 mM, 10.7 mM, 10.8 mM, 10.9 mM, 11 mM, 11.1 mM, 11.2 mM, 11.3 mM, 11.4 mM, 11.5 mM, 11.6 mM, 11.7 mM, 11.8 mM, 11.9 mM, 12 mM, 12.1 mM, 12.2 mM, 12.3 mM, 12.4 mM, 12.5 mM, 12.6 mM, 12.7 mM, 12.8 mM, 12.9 mM, 13 mM, 13.1 mM, 13.2 mM, 13.3 mM, 13.4 mM, 13.5 mM, 13.6 mM, 13.7 mM, 13.8 mM, 13.9 mM, 14 mM, 14.1 mM, 14.2 mM, 14.3 mM, 14.4 mM, 14.5 mM, 14.6 mM, 14.7 mM, 14.8 mM, 14.9 mM, 15 mM, 15.1 mM, 15.2 mM, 15.3 mM, 15.4 mM, 15.5 mM, 15.6 mM, 15.7 mM, 15.8 mM, 15.9 mM, 16 mM, 16.1 mM, 16.2 mM, 16.3 mM, 16.4 mM, 16.5 mM, 16.6 mM, 16.7 mM, 16.8 mM, 16.9 mM, 17 mM, 17.1 mM, 17.2 mM, 17.3 mM, 17.4 mM, 17.5 mM, 17.6 mM, 17.7 mM, 17.8 mM, 17.9 mM, 18 mM, 18.1 mM, 18.2 mM, 18.3 mM, 18.4 mM, 18.5 mM, 18.6 mM, 18.7 mM, 18.8 mM, 18.9 mM, 19 mM, 19.1 mM, 19.2 mM, 19.3 mM, 19.4 mM, 19.5 mM, 19.6 mM, 19.7 mM, 19.8 mM, 19.9 mM, or 20 mM, or any concentration therebetween. In some embodiments, the total amount of L-histidine in the pharmaceutical composition is about 10 mM. In some embodiments, the total amount of L-histidine in the pharmaceutical composition is in the form L-histidine. In some embodiments, the total amount of L-histidine in the pharmaceutical composition is in the form of L-histidine monohydrochloride monohydrate. In some embodiments, the total amount of L-histidine in the pharmaceutical composition is in the forms of both L-histidine and L-histidine monohydrochloride monohydrate. In some embodiments, the molar ratio of L-histidine to L-histidine monohydrochloride monohydrate is about 1:1, 1:2, 1:3, 1:4, 1:5, 1:6, 2:1, 2:3, 2:5, 3:1, 3:2, 3:4, 3:5, 4:1, 4:3, 4:5, 5:1, 5:2, 5:3, 5:4, 5:6, 6:1, or 6:5. In some embodiments, the molar ratio of L-histidine to L-histidine monohydrochloride monohydrate is about 3:2. In some embodiments, the pharmaceutical composition comprises sucrose in an amount of about 1% (w/v), 1.5% (w/v), 2% (w/v), 2.5% (w/v), 3% (w/v), 3.5% (w/v), 4% (w/v), 4.5% (w/v), 5% (w/v), 5.5% (w/v), 6% (w/v), 6.5% (w/v), 7% (w/v), 7.5% (w/v), 8% (w/v), 8.5% (w/v), 9% (w/v), 9.5% (w/v), 10% (w/v), 10.5% (w/v), 11% (w/v), 11.5% (w/v), 12% (w/v), 12.5% (w/v), 13% (w/v), 13.5% (w/v), 14% (w/v), 14.5% (w/v), 15% (w/v), 15.5% (w/v), 16% (w/v), 16.5% (w/v), 17% (w/v), 17.5% (w/v), 18% (w/v), 18.5% (w/v), 19% (w/v), 19.5% (w/v), or 20% (w/v), or any concentration therebetween. In some embodiments, the pharmaceutical composition comprises sucrose in an amount of about 8% (w/v).

In some embodiments, the pharmaceutical composition comprises polysorbate 20 in an amount of at least 0.001% (w/v), 0.0015% (w/v), 0.002% (w/v), 0.0025% (w/v), 0.003% (w/v), 0.0035% (w/v), 0.004% (w/v), 0.0045% (w/v), 0.005% (w/v), 0.0055% (w/v), 0.006% (w/v), 0.0065% (w/v), 0.007% (w/v), 0.0075% (w/v), 0.008% (w/v), 0.0085% (w/v), 0.009% (w/v), 0.0095% (w/v), 0.01% (w/v), 0.0105% (w/v), 0.011% (w/v), 0.0115% (w/v), 0.012% (w/v), 0.0125% (w/v), 0.013% (w/v), 0.0135% (w/v), 0.014% (w/v), 0.0145% (w/v), 0.015% (w/v), 0.0155% (w/v), 0.016% (w/v), 0.0165% (w/v), 0.017% (w/v), 0.0175% (w/v), 0.018% (w/v), 0.0185% (w/v), 0.019% (w/v), 0.0195% (w/v), 0.02% (w/v), 0.0205% (w/v), 0.021% (w/v), 0.0215% (w/v), 0.022% (w/v), 0.0225% (w/v), 0.023% (w/v), 0.0235% (w/v), 0.024% (w/v), 0.0245% (w/v), 0.025% (w/v), 0.0255% (w/v), 0.026% (w/v), 0.0265% (w/v), 0.027% (w/v), 0.0275% (w/v), 0.028% (w/v), 0.0285% (w/v), 0.029% (w/v), 0.0295% (w/v), 0.03% (w/v), 0.0305% (w/v), 0.031% (w/v), 0.0315% (w/v), 0.032% (w/v), 0.0325% (w/v), 0.033% (w/v), 0.0335% (w/v), 0.034% (w/v), 0.0345% (w/v), 0.035% (w/v), 0.0355% (w/v), 0.036% (w/v), 0.0365% (w/v), 0.037% (w/v), 0.0375% (w/v), 0.038% (w/v), 0.0385% (w/v), 0.039% (w/v), 0.0395% (w/v), 0.04% (w/v), 0.0405% (w/v), 0.041% (w/v), 0.0415% (w/v), 0.042% (w/v), 0.0425% (w/v), 0.043% (w/v), 0.0435% (w/v), 0.044% (w/v), 0.0445% (w/v), 0.045% (w/v), 0.0455% (w/v), 0.046% (w/v), 0.0465% (w/v), 0.047% (w/v), 0.0475% (w/v), 0.048% (w/v), 0.0485% (w/v), 0.049% (w/v), 0.0495% (w/v), 0.05% (w/v), 0.055% (w/v), 0.06% (w/v), 0.065% (w/v), 0.07% (w/v), 0.075% (w/v), 0.08% (w/v), 0.085% (w/v), 0.09% (w/v), 0.095% (w/v), or about 0.1% (w/v), or any concentration therebetween. In some embodiments, the pharmaceutical composition comprises polysorbate 20 in an amount of at least 0.01% (w/v). In some embodiments, the pharmaceutical composition comprises polysorbate 20 in an amount of about 0.01% (w/v). In some embodiments, the pharmaceutical composition comprises about 6 mM L-histidine. In some embodiments, the pharmaceutical composition comprises about 4 mM L-histidine monohydrochloride monohydrate. In some embodiments, the pharmaceutical composition comprises about 8% (w/v) sucrose. In some embodiments, the pharmaceutical composition comprises about 0.01% (w/v) polysorbate 20. In some embodiments, the pharmaceutical composition comprises about 6 mM L-histidine, about 4 mM L-histidine monohydrochloride monohydrate, about 8% (w/v) sucrose, and about 0.01% (w/v) polysorbate 20.

In some embodiments, the pharmaceutical composition comprises the isolated recombinant polypeptide complex in an amount of about 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1 mg/ml, 1.1 mg/ml, 1.2 mg/ml, 1.3 mg/ml, 1.4 mg/ml, 1.5 mg/ml, 1.6 mg/ml, 1.7 mg/ml, 1.8 mg/ml, 1.9 mg/ml, 2 mg/ml, 2.1 mg/ml, 2.2 mg/ml, 2.3 mg/ml, 2.4 mg/ml, 2.5 mg/ml, 2.6 mg/ml, 2.7 mg/ml, 2.8 mg/ml, 2.9 mg/ml, 3 mg/ml, 3.1 mg/ml, 3.2 mg/ml, 3.3 mg/ml, 3.4 mg/ml, 3.5 mg/ml, 3.6 mg/ml, 3.7 mg/ml, 3.8 mg/ml, 3.9 mg/ml, 4 mg/ml, 4.1 mg/ml, 4.2 mg/ml, 4.3 mg/ml, 4.4 mg/ml, 4.5 mg/ml, 4.6 mg/ml, 4.7 mg/ml, 4.8 mg/ml, 4.9 mg/ml, 5 mg/ml, 5.1 mg/ml, 5.2 mg/ml, 5.3 mg/ml, 5.4 mg/ml, 5.5 mg/ml, 5.6 mg/ml, 5.7 mg/ml, 5.8 mg/ml, 5.9 mg/ml, 6 mg/ml, 6.1 mg/ml, 6.2 mg/ml, 6.3 mg/ml, 6.4 mg/ml, 6.5 mg/ml, 6.6 mg/ml, 6.7 mg/ml, 6.8 mg/ml, 6.9 mg/ml, 7 mg/ml, 7.1 mg/ml, 7.2 mg/ml, 7.3 mg/ml, 7.4 mg/ml, 7.5 mg/ml, 7.6 mg/ml, 7.7 mg/ml, 7.8 mg/ml, 7.9 mg/ml, 8 mg/ml, 8.1 mg/ml, 8.2 mg/ml, 8.3 mg/ml, 8.4 mg/ml, 8.5 mg/ml, 8.6 mg/ml, 8.7 mg/ml, 8.8 mg/ml, 8.9 mg/ml, 9 mg/ml, 9.1 mg/ml, 9.2 mg/ml, 9.3 mg/ml, 9.4 mg/ml, 9.5 mg/ml, 9.6 mg/ml, 9.7 mg/ml, 9.8 mg/ml, 9.9 mg/ml, 10 mg/ml, 10.1 mg/ml, 10.2 mg/ml, 10.3 mg/ml, 10.4 mg/ml, 10.5 mg/ml, 10.6 mg/ml, 10.7 mg/ml, 10.8 mg/ml, 10.9 mg/ml, 11 mg/ml, 11.1 mg/ml, 11.2 mg/ml, 11.3 mg/ml, 11.4 mg/ml, 11.5 mg/ml, 11.6 mg/ml, 11.7 mg/ml, 11.8 mg/ml, 11.9 mg/ml, 12 mg/ml, 12.1 mg/ml, 12.2 mg/ml, 12.3 mg/ml, 12.4 mg/ml, 12.5 mg/ml, 12.6 mg/ml, 12.7 mg/ml, 12.8 mg/ml, 12.9 mg/ml, 13 mg/ml, 13.1 mg/ml, 13.2 mg/ml, 13.3 mg/ml, 13.4 mg/ml, 13.5 mg/ml, 13.6 mg/ml, 13.7 mg/ml, 13.8 mg/ml, 13.9 mg/ml, 14 mg/ml, 14.1 mg/ml, 14.2 mg/ml, 14.3 mg/ml, 14.4 mg/ml, 14.5 mg/ml, 14.6 mg/ml, 14.7 mg/ml, 14.8 mg/ml, 14.9 mg/ml, 15 mg/ml, 15.1 mg/ml, 15.2 mg/ml, 15.3 mg/ml, 15.4 mg/ml, 15.5 mg/ml, 15.6 mg/ml, 15.7 mg/ml, 15.8 mg/ml, 15.9 mg/ml, 16 mg/ml, 16.1 mg/ml, 16.2 mg/ml, 16.3 mg/ml, 16.4 mg/ml, 16.5 mg/ml, 16.6 mg/ml, 16.7 mg/ml, 16.8 mg/ml, 16.9 mg/ml, 17 mg/ml, 17.1 mg/ml, 17.2 mg/ml, 17.3 mg/ml, 17.4 mg/ml, 17.5 mg/ml, 17.6 mg/ml, 17.7 mg/ml, 17.8 mg/ml, 17.9 mg/ml, 18 mg/ml, 18.1 mg/ml, 18.2 mg/ml, 18.3 mg/ml, 18.4 mg/ml, 18.5 mg/ml, 18.6 mg/ml, 18.7 mg/ml, 18.8 mg/ml, 18.9 mg/ml, 19 mg/ml, 19.1 mg/ml, 19.2 mg/ml, 19.3 mg/ml, 19.4 mg/ml, 19.5 mg/ml, 19.6 mg/ml, 19.7 mg/ml, 19.8 mg/ml, 19.9 mg/ml, or 20 mg/ml, or any concentration therebetween. In some embodiments, the pharmaceutical composition comprises about 2 mg/ml of the isolated recombinant polypeptide complex.

In some embodiments, the pharmaceutical composition comprises a pH of about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or about 9.0, or any value therebetween. In some embodiments, the pharmaceutical composition comprises a pH between about 5 and about 7. In some embodiments, the pharmaceutical composition comprises a pH of about 6.3.

Disclosed herein, in another aspect, is a pharmaceutical composition comprising an isolated polypeptide comprising an amino acid sequence with at least 80% sequence identity to the amino acid sequence of SEQ ID NO: 5 and the isolated polypeptide is 484 amino acids in length, and pharmaceutically acceptable excipients.

Disclosed herein, in another aspect, is a pharmaceutical composition comprising an isolated polypeptide comprising an amino acid sequence with at least 81% sequence identity to the amino acid sequence of SEQ ID NO: 5 and the isolated polypeptide is 484 amino acids in length, and pharmaceutically acceptable excipients.

Disclosed herein, in another aspect, is a pharmaceutical composition comprising an isolated polypeptide comprising an amino acid sequence with at least 82% sequence identity to the amino acid sequence of SEQ ID NO: 5 and the isolated polypeptide is 484 amino acids in length, and pharmaceutically acceptable excipients.

Disclosed herein, in another aspect, is a pharmaceutical composition comprising an isolated polypeptide comprising an amino acid sequence with at least 83% sequence identity to the amino acid sequence of SEQ ID NO: 5 and the isolated polypeptide is 484 amino acids in length, and pharmaceutically acceptable excipients.

Disclosed herein, in another aspect, is a pharmaceutical composition comprising an isolated polypeptide comprising an amino acid sequence with at least 84% sequence identity to the amino acid sequence of SEQ ID NO: 5 and the isolated polypeptide is 484 amino acids in length, and pharmaceutically acceptable excipients.

Disclosed herein, in another aspect, is a pharmaceutical composition comprising an isolated polypeptide comprising an amino acid sequence with at least 85% sequence identity to the amino acid sequence of SEQ ID NO: 5 and the isolated polypeptide is 484 amino acids in length, and pharmaceutically acceptable excipients.

Disclosed herein, in another aspect, is a pharmaceutical composition comprising an isolated polypeptide comprising an amino acid sequence with at least 86% sequence identity to the amino acid sequence of SEQ ID NO: 5 and the isolated polypeptide is 484 amino acids in length, and pharmaceutically acceptable excipients.

Disclosed herein, in another aspect, is a pharmaceutical composition comprising an isolated polypeptide comprising an amino acid sequence with at least 87% sequence identity to the amino acid sequence of SEQ ID NO: 5 and the isolated polypeptide is 484 amino acids in length, and pharmaceutically acceptable excipients.

Disclosed herein, in another aspect, is a pharmaceutical composition comprising an isolated polypeptide comprising an amino acid sequence with at least 88% sequence identity to the amino acid sequence of SEQ ID NO: 5 and the isolated polypeptide is 484 amino acids in length, and pharmaceutically acceptable excipients.

Disclosed herein, in another aspect, is a pharmaceutical composition comprising an isolated polypeptide comprising an amino acid sequence with at least 89% sequence identity to the amino acid sequence of SEQ ID NO: 5 and the isolated polypeptide is 484 amino acids in length, and pharmaceutically acceptable excipients.

Disclosed herein, in another aspect, is a pharmaceutical composition comprising an isolated polypeptide comprising an amino acid sequence with at least 90% sequence identity to the amino acid sequence of SEQ ID NO: 5 and the isolated polypeptide is 484 amino acids in length, and pharmaceutically acceptable excipients.

Disclosed herein, in another aspect, is a pharmaceutical composition comprising an isolated polypeptide comprising an amino acid sequence with at least 91% sequence identity to the amino acid sequence of SEQ ID NO: 5 and the isolated polypeptide is 484 amino acids in length, and pharmaceutically acceptable excipients.

Disclosed herein, in another aspect, is a pharmaceutical composition comprising an isolated polypeptide comprising an amino acid sequence with at least 92% sequence identity to the amino acid sequence of SEQ ID NO: 5 and the isolated polypeptide is 484 amino acids in length, and pharmaceutically acceptable excipients.

Disclosed herein, in another aspect, is a pharmaceutical composition comprising an isolated polypeptide comprising an amino acid sequence with at least 93% sequence identity to the amino acid sequence of SEQ ID NO: 5 and the isolated polypeptide is 484 amino acids in length, and pharmaceutically acceptable excipients.

Disclosed herein, in another aspect, is a pharmaceutical composition comprising an isolated polypeptide comprising an amino acid sequence with at least 94% sequence identity to the amino acid sequence of SEQ ID NO: 5 and the isolated polypeptide is 484 amino acids in length, and pharmaceutically acceptable excipients.

Disclosed herein, in another aspect, is a pharmaceutical composition comprising an isolated polypeptide comprising an amino acid sequence with at least 95% sequence identity to the amino acid sequence of SEQ ID NO: 5 and the isolated polypeptide is 484 amino acids in length, and pharmaceutically acceptable excipients.

Disclosed herein, in another aspect, is a pharmaceutical composition comprising an isolated polypeptide comprising an amino acid sequence with at least 96% sequence identity to the amino acid sequence of SEQ ID NO: 5 and the isolated polypeptide is 484 amino acids in length, and pharmaceutically acceptable excipients.

Disclosed herein, in another aspect, is a pharmaceutical composition comprising an isolated polypeptide comprising an amino acid sequence with at least 97% sequence identity to the amino acid sequence of SEQ ID NO: 5 and the isolated polypeptide is 484 amino acids in length, and pharmaceutically acceptable excipients.

Disclosed herein, in another aspect, is a pharmaceutical composition comprising an isolated polypeptide comprising an amino acid sequence with at least 98% sequence identity to the amino acid sequence of SEQ ID NO: 5 and the isolated polypeptide is 484 amino acids in length, and pharmaceutically acceptable excipients.

Disclosed herein, in another aspect, is a pharmaceutical composition comprising an isolated polypeptide comprising an amino acid sequence with at least 99% sequence identity to the amino acid sequence of SEQ ID NO: 5 and the isolated polypeptide is 484 amino acids in length, and pharmaceutically acceptable excipients.

Disclosed herein, in another aspect, is a pharmaceutical composition comprising an isolated polypeptide comprising the amino acid sequence of the amino acid sequence of SEQ ID NO: 5 and the isolated polypeptide is 484 amino acids in length, and pharmaceutically acceptable excipients.

Disclosed herein, in another aspect, is a pharmaceutical composition comprising an isolated polypeptide comprising an amino acid sequence with at least 80% sequence identity to the amino acid sequence of SEQ ID NO: 6 and the isolated polypeptide is 476 amino acids in length, and pharmaceutically acceptable excipients.

Disclosed herein, in another aspect, is a pharmaceutical composition comprising an isolated polypeptide comprising an amino acid sequence with at least 81% sequence identity to the amino acid sequence of SEQ ID NO: 6 and the isolated polypeptide is 476 amino acids in length, and pharmaceutically acceptable excipients.

Disclosed herein, in another aspect, is a pharmaceutical composition comprising an isolated polypeptide comprising an amino acid sequence with at least 82% sequence identity to the amino acid sequence of SEQ ID NO: 6 and the isolated polypeptide is 476 amino acids in length, and pharmaceutically acceptable excipients.

Disclosed herein, in another aspect, is a pharmaceutical composition comprising an isolated polypeptide comprising an amino acid sequence with at least 83% sequence identity to the amino acid sequence of SEQ ID NO: 6 and the isolated polypeptide is 476 amino acids in length, and pharmaceutically acceptable excipients.

Disclosed herein, in another aspect, is a pharmaceutical composition comprising an isolated polypeptide comprising an amino acid sequence with at least 84% sequence identity to the amino acid sequence of SEQ ID NO: 6 and the isolated polypeptide is 476 amino acids in length, and pharmaceutically acceptable excipients.

Disclosed herein, in another aspect, is a pharmaceutical composition comprising an isolated polypeptide comprising an amino acid sequence with at least 85% sequence identity to the amino acid sequence of SEQ ID NO: 6 and the isolated polypeptide is 476 amino acids in length, and pharmaceutically acceptable excipients.

Disclosed herein, in another aspect, is a pharmaceutical composition comprising an isolated polypeptide comprising an amino acid sequence with at least 86% sequence identity to the amino acid sequence of SEQ ID NO: 6 and the isolated polypeptide is 476 amino acids in length, and pharmaceutically acceptable excipients.

Disclosed herein, in another aspect, is a pharmaceutical composition comprising an isolated polypeptide comprising an amino acid sequence with at least 87% sequence identity to the amino acid sequence of SEQ ID NO: 6 and the isolated polypeptide is 476 amino acids in length, and pharmaceutically acceptable excipients.

Disclosed herein, in another aspect, is a pharmaceutical composition comprising an isolated polypeptide comprising an amino acid sequence with at least 88% sequence identity to the amino acid sequence of SEQ ID NO: 6 and the isolated polypeptide is 476 amino acids in length, and pharmaceutically acceptable excipients.

Disclosed herein, in another aspect, is a pharmaceutical composition comprising an isolated polypeptide comprising an amino acid sequence with at least 89% sequence identity to the amino acid sequence of SEQ ID NO: 6 and the isolated polypeptide is 476 amino acids in length, and pharmaceutically acceptable excipients.

Disclosed herein, in another aspect, is a pharmaceutical composition comprising an isolated polypeptide comprising an amino acid sequence with at least 90% sequence identity to the amino acid sequence of SEQ ID NO: 6 and the isolated polypeptide is 476 amino acids in length, and pharmaceutically acceptable excipients.

Disclosed herein, in another aspect, is a pharmaceutical composition comprising an isolated polypeptide comprising an amino acid sequence with at least 91% sequence identity to the amino acid sequence of SEQ ID NO: 6 and the isolated polypeptide is 476 amino acids in length, and pharmaceutically acceptable excipients.

Disclosed herein, in another aspect, is a pharmaceutical composition comprising an isolated polypeptide comprising an amino acid sequence with at least 92% sequence identity to the amino acid sequence of SEQ ID NO: 6 and the isolated polypeptide is 476 amino acids in length, and pharmaceutically acceptable excipients.

Disclosed herein, in another aspect, is a pharmaceutical composition comprising an isolated polypeptide comprising an amino acid sequence with at least 93% sequence identity to the amino acid sequence of SEQ ID NO: 6 and the isolated polypeptide is 476 amino acids in length, and pharmaceutically acceptable excipients.

Disclosed herein, in another aspect, is a pharmaceutical composition comprising an isolated polypeptide comprising an amino acid sequence with at least 94% sequence identity to the amino acid sequence of SEQ ID NO: 6 and the isolated polypeptide is 476 amino acids in length, and pharmaceutically acceptable excipients.

Disclosed herein, in another aspect, is a pharmaceutical composition comprising an isolated polypeptide comprising an amino acid sequence with at least 95% sequence identity to the amino acid sequence of SEQ ID NO: 6 and the isolated polypeptide is 476 amino acids in length, and pharmaceutically acceptable excipients.

Disclosed herein, in another aspect, is a pharmaceutical composition comprising an isolated polypeptide comprising an amino acid sequence with at least 96% sequence identity to the amino acid sequence of SEQ ID NO: 6 and the isolated polypeptide is 476 amino acids in length, and pharmaceutically acceptable excipients.

Disclosed herein, in another aspect, is a pharmaceutical composition comprising an isolated polypeptide comprising an amino acid sequence with at least 97% sequence identity to the amino acid sequence of SEQ ID NO: 6 and the isolated polypeptide is 476 amino acids in length, and pharmaceutically acceptable excipients.

Disclosed herein, in another aspect, is a pharmaceutical composition comprising an isolated polypeptide comprising an amino acid sequence with at least 98% sequence identity to the amino acid sequence of SEQ ID NO: 6 and the isolated polypeptide is 476 amino acids in length, and pharmaceutically acceptable excipients.

Disclosed herein, in another aspect, is a pharmaceutical composition comprising an isolated polypeptide comprising an amino acid sequence with at least 99% sequence identity to the amino acid sequence of SEQ ID NO: 6 and the isolated polypeptide is 476 amino acids in length, and pharmaceutically acceptable excipients.

Disclosed herein, in another aspect, is a pharmaceutical composition comprising an isolated polypeptide comprising the amino acid sequence of the amino acid sequence of SEQ ID NO: 6 and the isolated polypeptide is 476 amino acids in length, and pharmaceutically acceptable excipients.

Disclosed herein, in another aspect, is method for treating cancer comprising administering to a subject in need thereof an effective amount of a pharmaceutical composition disclosed herein.

In some embodiments, the cancer comprises mCRPC. In some embodiments, the subject is human.

### Kits

Provided herein, in some embodiments, is a kit comprising a recombinant polypeptide (such as any described herein) or a composition (such as any described herein), a container, and a label or package insert on or associated with the container.

### Methods of Treatment

In some embodiments, are methods of treating cancer in a subject need in need thereof comprising administering to the subject an isolated recombinant polypeptide complex as described herein. In some embodiments, the cancer has cells that express PSMA. In some instances, the cancer is a solid tumor cancer. In some embodiments, the cancer is lung, breast (e.g. HER2+; ER/PR+; TNBC), cervical, ovarian, colorectal, pancreatic or gastric.

In some embodiments, are methods of treating prostate cancer in a subject in need thereof comprising administering to the subject an isolated recombinant polypeptide complex as described herein. In some embodiments, are methods of treating metastatic castrate-resistant prostate cancer (mCRPC) in a subject need in need thereof comprising administering to the subject an isolated recombinant polypeptide complex as described herein.

Provided herein, in some embodiments, are methods of treating metastatic castration-resistance prostate cancer (mCRPC) in a subject in need thereof comprising administering to the subject a recombinant polypeptide (such as any described herein) or a formulation (such as any described herein). In some embodiments, the subject has at least one symptom of mCRPC. In some embodiments, the subject has received a diagnosis of mCRPC.

For administration to a subject, the recombinant polypeptide as disclosed herein, may be provided in a pharmaceutical composition together with one or more pharmaceutically acceptable carriers or excipients. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms may be ensured by the inclusion of various antibacterial and antifungal agents.

The pharmaceutical composition may be in any suitable form, (depending upon the desired method of administration). It may be provided in unit dosage form, may be provided in a sealed container and may be provided as part of a kit. Such a kit may include instructions for use. It may include a plurality of said unit dosage forms.

The pharmaceutical composition may be adapted for administration by any appropriate route, including a parenteral (e.g., subcutaneous, intramuscular, or intravenous) route. Such compositions may be prepared by any method known in the art of pharmacy, for example by mixing the active ingredient with the carrier(s) or excipient(s) under sterile conditions. In some embodiments, a pharmaceutical composition disclosed herein is administered intravenously to a subject in need thereof.

Dosages of the substances of the present disclosure can vary between wide limits, depending upon the disease or disorder to be treated, the age and condition of the individual to be treated, etc. and a physician will ultimately determine appropriate dosages to be used.

Disclosed herein, in another aspect is a method for treating cancer comprising administering to a subject in need thereof an effective amount of an isolated recombinant polypeptide complex disclosed herein. In some embodiments, the method comprises administering the isolated recombinant polypeptide complex to the subject at a first dose and a second dose. In some embodiments, the second dose is equal to or higher than the first dose. In some embodiments, the first or second dose is at least 100 μg. In some embodiments, the first or second dose is about 100 μg.

In some embodiments, the method comprises administering the isolated recombinant polypeptide complex to the subject at a first dose, a second dose and a third dose. In some embodiments, the second dose is equal to or higher than the first dose. In some embodiments, the third dose is equal to or higher than the second dose. In some embodiments, the first, second, or third dose is at least 100 μg. In some embodiments, the first, second, or third dose is about 100 μg.

In some embodiments, the method comprises a first treatment course and a second treatment course. In some embodiments, the first dose is administered to the subject in the first treatment course. In some embodiments, the second dose is administered to the subject in the second treatment course. In some embodiments, the method comprises a 21-day treatment course. In some embodiments, the first dose is administered to the subject in the first week of the treatment course. In some embodiments, the second dose is administered to the subject in the second week of the treatment course. In some embodiments, the third dose is administered to the subject in the third week of the treatment course. In some embodiments, the method comprises a first 21-day treatment course and a second 21-day treatment course. In some embodiments, the first dose of the second 21-day treatment course is equal to or higher than the first dose of the first 21-day treatment course. In some embodiments, the second dose of the second 21-day treatment course is equal to or higher than the second dose of the first 21-day treatment course. In some embodiments, the third dose of the second 21-day treatment course is equal to or higher than the third dose of the first 21-day treatment course. In some embodiments, the method comprises a 21-day treatment cycle comprising: (a) administering the isolated recombinant polypeptide complex to the subject at a first dose in the first week of the treatment cycle; (b) administering the isolated recombinant polypeptide complex to the subject at a second dose in the second week of the treatment cycle; and (c) administering the isolated recombinant polypeptide complex to the subject at a third dose in the third week of the treatment cycle. In some embodiments, the method comprises: (a) administering the isolated recombinant polypeptide complex to the subject at the first dose on the first day of the 21-day treatment cycle; (b) administering the isolated recombinant polypeptide complex to the subject at the second dose on the eighth day of the 21-day treatment cycle; and (c) administering the isolated recombinant polypeptide complex to the subject at the third dose on the fifteenth day of the 21-day treatment cycle. In some embodiments, the treatment cycle is repeated 6 times over 18 weeks.

In some embodiments, the first, second, or third dose is at least 100 μg. In some embodiments, the first, second, or third dose is about 100 μg. In some embodiments, the second dose is equal to or higher than the first dose. In some embodiments, the third dose is equal to or higher than the second dose. In some embodiments, the third dose is equal to or higher than the first dose.

In some embodiments, the method comprises a first 21-day treatment cycle and a second 21-day treatment cycle. In some embodiments, the first dose of the second 21-day treatment cycle is equal to or higher than the first dose of the first 21-day treatment cycle. In some embodiments, the second dose of the second 21-day treatment cycle is equal to or higher than the second dose of the first 21-day treatment cycle. In some embodiments, the third dose of the second 21-day treatment cycle is equal to or higher than the third dose of the 21-day first treatment cycle. In some embodiments, the administering comprises administering through intravenous infusion. In some embodiments, the cancer comprises prostate cancer. In some embodiments, the cancer comprises mCRPC. In some embodiments, the method further comprises treating the subject with a therapy for an infusion-related reaction before the administering. In some embodiments, the therapy for an infusion-related reaction comprises an antipyretic drug, an antihistamine drug, an antiemetic drug, or a corticosteroid. In some embodiments, the therapy for an infusion-related reaction comprises acetaminophen, paracetamol, or diphenhydramine. In some embodiments, the method further comprises treating the subject with a corticosteroid before the step of (a) administering the isolated recombinant polypeptide complex to the subject at a first dose in the first week of the 21-day treatment cycle. In some embodiments, the method further comprises treating the subject with a corticosteroid before the step of (a) administering the isolated recombinant polypeptide complex to the subject at the first dose on the first day of the 21-day treatment cycle. In some embodiments, the method further comprises treating the subject with a therapy for cytokine release syndrome (CRS) before or after the administering. In some embodiments, the therapy for CRS comprises an intravenous hydration procedure, an oxygen treatment, a corticosteroid, an immunosuppressant, a vasopressor, or an antiepileptic drug. In some embodiments, the oxygen treatment comprises mechanical ventilation. In some embodiments, the immunosuppressant comprises an IL-6 receptor inhibitor. In some embodiments, the IL-6 receptor inhibitor comprises tocilizumab. In some embodiments, the therapy for CRS comprises a high dose of a corticosteroid.

In some embodiments, the isolated recombinant polypeptide complex is cleaved by a tumor specific protease to generate an enzymatic product of the isolated recombinant polypeptide complex after the administering. In some embodiments, the tumor specific protease comprises two or more proteases. In some embodiments, the isolated recombinant polypeptide complex is cleaved by a first protease of the two or more proteases to generate a first metabolic product of the isolated recombinant polypeptide complex. In some embodiments, the isolated recombinant polypeptide complex is cleaved by a second protease of the two or more proteases to generate a second metabolic product of the isolated recombinant polypeptide complex. In some embodiments, the first protease comprises a serine protease. In some embodiments, the second protease comprises a matrix metalloprotease. In some embodiments, the serine protease comprises recombinant human matriptase (MTSP1). In some embodiments, the matrix metalloprotease comprises recombinant human matrix metalloprotease 9 (MMP9).

In some embodiments, the first metabolic product of the isolated recombinant polypeptide complex comprises an amino acid sequence with at least 80% sequence identity to the amino acid sequence of SEQ ID NO: 5. In some embodiments, the first metabolic product of the isolated recombinant polypeptide complex comprises an amino acid sequence with at least 81% sequence identity to the amino acid sequence of SEQ ID NO: 5. In some embodiments, the first metabolic product of the isolated recombinant polypeptide complex comprises an amino acid sequence with at least 82% sequence identity to the amino acid sequence of SEQ ID NO: 5. In some embodiments, the first metabolic product of the isolated recombinant polypeptide complex comprises an amino acid sequence with at least 83% sequence identity to the amino acid sequence of SEQ ID NO: 5. In some embodiments, the first metabolic product of the isolated recombinant polypeptide complex comprises an amino acid sequence with at least 84% sequence identity to the amino acid sequence of SEQ ID NO: 5. In some embodiments, the first metabolic product of the isolated recombinant polypeptide complex comprises an amino acid sequence with at least 85% sequence identity to the amino acid sequence of SEQ ID NO: 5. In some embodiments, the first metabolic product of the isolated recombinant polypeptide complex comprises an amino acid sequence with at least 86% sequence identity to the amino acid sequence of SEQ ID NO: 5. In some embodiments, the first metabolic product of the isolated recombinant polypeptide complex comprises an amino acid sequence with at least 87% sequence identity to the amino acid sequence of SEQ ID NO: 5. In some embodiments, the first metabolic product of the isolated recombinant polypeptide complex comprises an amino acid sequence with at least 88% sequence identity to the amino acid sequence of SEQ ID NO: 5. In some embodiments, the first metabolic product of the isolated recombinant polypeptide complex comprises an amino acid sequence with at least 89% sequence identity to the amino acid sequence of SEQ ID NO: 5. In some embodiments, the first metabolic product of the isolated recombinant polypeptide complex comprises an amino acid sequence with at least 90% sequence identity to the amino acid sequence of SEQ ID NO: 5. In some embodiments, the first metabolic product of the isolated recombinant polypeptide complex comprises an amino acid sequence with at least 91% sequence identity to the amino acid sequence of SEQ ID NO: 5. In some embodiments, the first metabolic product of the isolated recombinant polypeptide complex comprises an amino acid sequence with at least 92% sequence identity to the amino acid sequence of SEQ ID NO: 5. In some embodiments, the first metabolic product of the isolated recombinant polypeptide complex comprises an amino acid sequence with at least 93% sequence identity to the amino acid sequence of SEQ ID NO: 5. In some embodiments, the first metabolic product of the isolated recombinant polypeptide complex comprises an amino acid sequence with at least 94% sequence identity to the amino acid sequence of SEQ ID NO: 5. In some embodiments, the first metabolic product of the isolated recombinant polypeptide complex comprises an amino acid sequence with at least 95% sequence identity to the amino acid sequence of SEQ ID NO: 5. In some embodiments, the first metabolic product of the isolated recombinant polypeptide complex comprises an amino acid sequence with at least 96% sequence identity to the amino acid sequence of SEQ ID NO: 5. In some embodiments, the first metabolic product of the isolated recombinant polypeptide complex comprises an amino acid sequence with at least 97% sequence identity to the amino acid sequence of SEQ ID NO: 5. In some embodiments, the first metabolic product of the isolated recombinant polypeptide complex comprises an amino acid sequence with at least 98% sequence identity to the amino acid sequence of SEQ ID NO: 5. In some embodiments, the first metabolic product of the isolated recombinant polypeptide complex comprises an amino acid sequence with at least 99% sequence identity to the amino acid sequence of SEQ ID NO: 5. In some embodiments, the first metabolic product of the isolated recombinant polypeptide complex comprises the amino acid sequence of SEQ ID NO: 5.

In some embodiments, the second metabolic product of the isolated recombinant polypeptide complex comprises an amino acid sequence with at least 80% sequence identity to the amino acid sequence of SEQ ID NO: 6. In some embodiments, the second metabolic product of the isolated recombinant polypeptide complex comprises an amino acid sequence with at least 81% sequence identity to the amino acid sequence of SEQ ID NO: 6. In some embodiments, the second metabolic product of the isolated recombinant polypeptide complex comprises an amino acid sequence with at least 82% sequence identity to the amino acid sequence of SEQ ID NO: 6. In some embodiments, the second metabolic product of the isolated recombinant polypeptide complex comprises an amino acid sequence with at least 83% sequence identity to the amino acid sequence of SEQ ID NO: 6. In some embodiments, the second metabolic product of the isolated recombinant polypeptide complex comprises an amino acid sequence with at least 84% sequence identity to the amino acid sequence of SEQ ID NO: 6. In some embodiments, the second metabolic product of the isolated recombinant polypeptide complex comprises an amino acid sequence with at least 85% sequence identity to the amino acid sequence of SEQ ID NO: 6. In some embodiments, the second metabolic product of the isolated recombinant polypeptide complex comprises an amino acid sequence with at least 86% sequence identity to the amino acid sequence of SEQ ID NO: 6. In some embodiments, the second metabolic product of the isolated recombinant polypeptide complex comprises an amino acid sequence with at least 87% sequence identity to the amino acid sequence of SEQ ID NO: 6. In some embodiments, the second metabolic product of the isolated recombinant polypeptide complex comprises an amino acid sequence with at least 88% sequence identity to the amino acid sequence of SEQ ID NO: 6. In some embodiments, the second metabolic product of the isolated recombinant polypeptide complex comprises an amino acid sequence with at least 89% sequence identity to the amino acid sequence of SEQ ID NO: 6. In some embodiments, the second metabolic product of the isolated recombinant polypeptide complex comprises an amino acid sequence with at least 90% sequence identity to the amino acid sequence of SEQ ID NO: 6. In some embodiments, the second metabolic product of the isolated recombinant polypeptide complex comprises an amino acid sequence with at least 91% sequence identity to the amino acid sequence of SEQ ID NO: 6. In some embodiments, the second metabolic product of the isolated recombinant polypeptide complex comprises an amino acid sequence with at least 92% sequence identity to the amino acid sequence of SEQ ID NO: 6. In some embodiments, the second metabolic product of the isolated recombinant polypeptide complex comprises an amino acid sequence with at least 93% sequence identity to the amino acid sequence of SEQ ID NO: 6. In some embodiments, the second metabolic product of the isolated recombinant polypeptide complex comprises an amino acid sequence with at least 94% sequence identity to the amino acid sequence of SEQ ID NO: 6. In some embodiments, the second metabolic product of the isolated recombinant polypeptide complex comprises an amino acid sequence with at least 95% sequence identity to the amino acid sequence of SEQ ID NO: 6. In some embodiments, the second metabolic product of the isolated recombinant polypeptide complex comprises an amino acid sequence with at least 96% sequence identity to the amino acid sequence of SEQ ID NO: 6. In some embodiments, the second metabolic product of the isolated recombinant polypeptide complex comprises an amino acid sequence with at least 97% sequence identity to the amino acid sequence of SEQ ID NO: 6. In some embodiments, the second metabolic product of the isolated recombinant polypeptide complex comprises an amino acid sequence with at least 98% sequence identity to the amino acid sequence of SEQ ID NO: 6. In some embodiments, the second metabolic product of the isolated recombinant polypeptide complex comprises an amino acid sequence with at least 99% sequence identity to the amino acid sequence of SEQ ID NO: 6. In some embodiments, the second metabolic product of the isolated recombinant polypeptide complex comprises the amino acid sequence of SEQ ID NO: 6.

In some embodiments, the subject is a non-human primate or human. In some embodiments, the non-human primate is cynomolgus monkey.

Disclosed herein, in another aspect, is an isolated polypeptide comprising an amino acid sequence with at least 80% sequence identity to the amino acid sequence of SEQ ID NO: 5 and the isolated polypeptide is 484 amino acids in length.

Disclosed herein, in another aspect, is an isolated polypeptide comprising an amino acid sequence with at least 81% sequence identity to the amino acid sequence of SEQ ID NO: 5 and the isolated polypeptide is 484 amino acids in length.

Disclosed herein, in another aspect, is an isolated polypeptide comprising an amino acid sequence with at least 82% sequence identity to the amino acid sequence of SEQ ID NO: 5 and the isolated polypeptide is 484 amino acids in length.

Disclosed herein, in another aspect, is an isolated polypeptide comprising an amino acid sequence with at least 83% sequence identity to the amino acid sequence of SEQ ID NO: 5 and the isolated polypeptide is 484 amino acids in length.

Disclosed herein, in another aspect, is an isolated polypeptide comprising an amino acid sequence with at least 84% sequence identity to the amino acid sequence of SEQ ID NO: 5 and the isolated polypeptide is 484 amino acids in length.

Disclosed herein, in another aspect, is an isolated polypeptide comprising an amino acid sequence with at least 85% sequence identity to the amino acid sequence of SEQ ID NO: 5 and the isolated polypeptide is 484 amino acids in length.

Disclosed herein, in another aspect, is an isolated polypeptide comprising an amino acid sequence with at least 86% sequence identity to the amino acid sequence of SEQ ID NO: 5 and the isolated polypeptide is 484 amino acids in length.

Disclosed herein, in another aspect, is an isolated polypeptide comprising an amino acid sequence with at least 87% sequence identity to the amino acid sequence of SEQ ID NO: 5 and the isolated polypeptide is 484 amino acids in length.

Disclosed herein, in another aspect, is an isolated polypeptide comprising an amino acid sequence with at least 88% sequence identity to the amino acid sequence of SEQ ID NO: 5 and the isolated polypeptide is 484 amino acids in length.

Disclosed herein, in another aspect, is an isolated polypeptide comprising an amino acid sequence with at least 89% sequence identity to the amino acid sequence of SEQ ID NO: 5 and the isolated polypeptide is 484 amino acids in length.

Disclosed herein, in another aspect, is an isolated polypeptide comprising an amino acid sequence with at least 90% sequence identity to the amino acid sequence of SEQ ID NO: 5 and the isolated polypeptide is 484 amino acids in length.

Disclosed herein, in another aspect, is an isolated polypeptide comprising an amino acid sequence with at least 91% sequence identity to the amino acid sequence of SEQ ID NO: 5 and the isolated polypeptide is 484 amino acids in length.

Disclosed herein, in another aspect, is an isolated polypeptide comprising an amino acid sequence with at least 92% sequence identity to the amino acid sequence of SEQ ID NO: 5 and the isolated polypeptide is 484 amino acids in length.

Disclosed herein, in another aspect, is an isolated polypeptide comprising an amino acid sequence with at least 93% sequence identity to the amino acid sequence of SEQ ID NO: 5 and the isolated polypeptide is 484 amino acids in length.

Disclosed herein, in another aspect, is an isolated polypeptide comprising an amino acid sequence with at least 94% sequence identity to the amino acid sequence of SEQ ID NO: 5 and the isolated polypeptide is 484 amino acids in length.

Disclosed herein, in another aspect, is an isolated polypeptide comprising an amino acid sequence with at least 95% sequence identity to the amino acid sequence of SEQ ID NO: 5 and the isolated polypeptide is 484 amino acids in length.

Disclosed herein, in another aspect, is an isolated polypeptide comprising an amino acid sequence with at least 96% sequence identity to the amino acid sequence of SEQ ID NO: 5 and the isolated polypeptide is 484 amino acids in length.

Disclosed herein, in another aspect, is an isolated polypeptide comprising an amino acid sequence with at least 97% sequence identity to the amino acid sequence of SEQ ID NO: 5 and the isolated polypeptide is 484 amino acids in length.

Disclosed herein, in another aspect, is an isolated polypeptide comprising an amino acid sequence with at least 98% sequence identity to the amino acid sequence of SEQ ID NO: 5 and the isolated polypeptide is 484 amino acids in length.

Disclosed herein, in another aspect, is an isolated polypeptide comprising an amino acid sequence with at least 99% sequence identity to the amino acid sequence of SEQ ID NO: 5 and the isolated polypeptide is 484 amino acids in length.

Disclosed herein, in another aspect, is an isolated polypeptide comprising the amino acid sequence of SEQ ID NO: 5 and the isolated polypeptide is 484 amino acids in length.

Disclosed herein, in another aspect, is an isolated polypeptide comprising an amino acid sequence with at least 80% sequence identity to the amino acid sequence of SEQ ID NO: 6 and the isolated polypeptide is 476 amino acids in length.

Disclosed herein, in another aspect, is an isolated polypeptide comprising an amino acid sequence with at least 81% sequence identity to the amino acid sequence of SEQ ID NO: 6 and the isolated polypeptide is 476 amino acids in length.

Disclosed herein, in another aspect, is an isolated polypeptide comprising an amino acid sequence with at least 82% sequence identity to the amino acid sequence of SEQ ID NO: 6 and the isolated polypeptide is 476 amino acids in length.

Disclosed herein, in another aspect, is an isolated polypeptide comprising an amino acid sequence with at least 83% sequence identity to the amino acid sequence of SEQ ID NO: 6 and the isolated polypeptide is 476 amino acids in length.

Disclosed herein, in another aspect, is an isolated polypeptide comprising an amino acid sequence with at least 84% sequence identity to the amino acid sequence of SEQ ID NO: 6 and the isolated polypeptide is 476 amino acids in length.

Disclosed herein, in another aspect, is an isolated polypeptide comprising an amino acid sequence with at least 85% sequence identity to the amino acid sequence of SEQ ID NO: 6 and the isolated polypeptide is 476 amino acids in length.

Disclosed herein, in another aspect, is an isolated polypeptide comprising an amino acid sequence with at least 86% sequence identity to the amino acid sequence of SEQ ID NO: 6 and the isolated polypeptide is 476 amino acids in length.

Disclosed herein, in another aspect, is an isolated polypeptide comprising an amino acid sequence with at least 87% sequence identity to the amino acid sequence of SEQ ID NO: 6 and the isolated polypeptide is 476 amino acids in length.

Disclosed herein, in another aspect, is an isolated polypeptide comprising an amino acid sequence with at least 88% sequence identity to the amino acid sequence of SEQ ID NO: 6 and the isolated polypeptide is 476 amino acids in length.

Disclosed herein, in another aspect, is an isolated polypeptide comprising an amino acid sequence with at least 89% sequence identity to the amino acid sequence of SEQ ID NO: 6 and the isolated polypeptide is 476 amino acids in length.

Disclosed herein, in another aspect, is an isolated polypeptide comprising an amino acid sequence with at least 90% sequence identity to the amino acid sequence of SEQ ID NO: 6 and the isolated polypeptide is 476 amino acids in length.

Disclosed herein, in another aspect, is an isolated polypeptide comprising an amino acid sequence with at least 91% sequence identity to the amino acid sequence of SEQ ID NO: 6 and the isolated polypeptide is 476 amino acids in length.

Disclosed herein, in another aspect, is an isolated polypeptide comprising an amino acid sequence with at least 92% sequence identity to the amino acid sequence of SEQ ID NO: 6 and the isolated polypeptide is 476 amino acids in length.

Disclosed herein, in another aspect, is an isolated polypeptide comprising an amino acid sequence with at least 93% sequence identity to the amino acid sequence of SEQ ID NO: 6 and the isolated polypeptide is 476 amino acids in length.

Disclosed herein, in another aspect, is an isolated polypeptide comprising an amino acid sequence with at least 94% sequence identity to the amino acid sequence of SEQ ID NO: 6 and the isolated polypeptide is 476 amino acids in length.

Disclosed herein, in another aspect, is an isolated polypeptide comprising an amino acid sequence with at least 95% sequence identity to the amino acid sequence of SEQ ID NO: 6 and the isolated polypeptide is 476 amino acids in length.

Disclosed herein, in another aspect, is an isolated polypeptide comprising an amino acid sequence with at least 96% sequence identity to the amino acid sequence of SEQ ID NO: 6 and the isolated polypeptide is 476 amino acids in length.

Disclosed herein, in another aspect, is an isolated polypeptide comprising an amino acid sequence with at least 97% sequence identity to the amino acid sequence of SEQ ID NO: 6 and the isolated polypeptide is 476 amino acids in length.

Disclosed herein, in another aspect, is an isolated polypeptide comprising an amino acid sequence with at least 98% sequence identity to the amino acid sequence of SEQ ID NO: 6 and the isolated polypeptide is 476 amino acids in length.

Disclosed herein, in another aspect, is an isolated polypeptide comprising an amino acid sequence with at least 99% sequence identity to the amino acid sequence of SEQ ID NO: 6 and the isolated polypeptide is 476 amino acids in length.

Disclosed herein, in another aspect, is an isolated polypeptide comprising the amino acid sequence of SEQ ID NO: 6 and the isolated polypeptide is 476 amino acids in length.

### Production of Antibodies

In some embodiments, polypeptides described herein (e.g., antibodies and its binding fragments) are produced using any method known in the art to be useful for the synthesis of polypeptides (e.g., antibodies), in particular, by chemical synthesis or by recombinant expression, and are preferably produced by recombinant expression techniques.

In some instances, an antibody or its binding fragment thereof is expressed recombinantly, and the nucleic acid encoding the antibody or its binding fragment is assembled from chemically synthesized oligonucleotides (e.g., as described in Kutmeier et al., 1994, BioTechniques 17:242), which involves the synthesis of overlapping oligonucleotides containing portions of the sequence encoding the antibody, annealing and ligation of those oligonucleotides, and then amplification of the ligated oligonucleotides by PCR

Alternatively, a nucleic acid molecule encoding an antibody is optionally generated from a suitable source (e.g., an antibody cDNA library, or cDNA library generated from any tissue or cells expressing the immunoglobulin) by PCR amplification using synthetic primers hybridizable to the 3′ and 5′ ends of the sequence or by cloning using an oligonucleotide probe specific for the particular gene sequence.

In some instances, an antibody or its binding is optionally generated by immunizing an animal, such as a mouse, to generate polyclonal antibodies or, more preferably, by generating monoclonal antibodies, e.g., as described by Kohler and Milstein (1975, Nature 256:495-497) or, as described by Kozbor et al. (1983, Immunology Today 4:72) or Cole et al. (1985 in Monoclonal Antibodies and Cancer Therapy, Alan R. Liss, Inc., pp. 77-96). Alternatively, a clone encoding at least the Fab portion of the antibody is optionally obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

In some embodiments, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity are used. A chimeric antibody is a molecule in which different portions are derived from different animal species, such as those having a variable region derived from a murine monoclonal antibody and a human immunoglobulin constant region.

In some embodiments, techniques described for the production of single chain antibodies (U.S. Pat. No. 4,694,778; Bird, 1988, Science 242:423-42; Huston et al., 1988, Proc. Natl. Acad. Sci. USA 85:5879-5883; and Ward et al., 1989, Nature 334:544-54) are adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli are also optionally used (Skerra et al., 1988, Science 242:1038-1041).

In some embodiments, an expression vector comprising the nucleotide sequence of an antibody or fragment thereof or the nucleotide sequence of an antibody or fragment thereof is transferred to a host cell by conventional techniques (e.g., electroporation, liposomal transfection, and calcium phosphate precipitation), and the transfected cells are then cultured by conventional techniques to produce the antibody. In specific embodiments, the expression of the antibody is regulated by a constitutive, an inducible or a tissue, specific promoter.

In some embodiments, a variety of host-expression vector systems is utilized to express an antibody, or its binding fragment described herein. Such host-expression systems represent vehicles by which the coding sequences of the antibody is produced and subsequently purified, but also represent cells that are, when transformed or transfected with the appropriate nucleotide coding sequences, express an antibody or its binding fragment in situ. These include, but are not limited to, microorganisms such as bacteria (e.g., E. coli and B. subtilis) transformed with recombinant bacteriophage DNA, plasmid DNA or cosmid DNA expression vectors containing an antibody or its binding fragment coding sequences; yeast (e.g., Saccharomyces Pichia) transformed with recombinant yeast expression vectors containing an antibody or its binding fragment coding sequences; insect cell systems infected with recombinant virus expression vectors (e.g., baculovirus) containing an antibody or its binding fragment coding sequences; plant cell systems infected with recombinant virus expression vectors (e.g., cauliflower mosaic virus (CaMV) and tobacco mosaic virus (TMV)) or transformed with recombinant plasmid expression vectors (e.g., Ti plasmid) containing an antibody or its binding fragment coding sequences; or mammalian cell systems (e.g., COS, CHO, BH, HEK293, 293T, 3T3 cells) harboring recombinant expression constructs containing promoters derived from the genome of mammalian cells (e.g., metallothionein promoter) or from mammalian viruses (e.g. the adenovirus late promoter; the vaccinia virus 7.5K promoter).

For long-term, high-yield production of recombinant proteins, stable expression is preferred. In some instances, cell lines that stably express an antibody are optionally engineered. Rather than using expression vectors that contain viral origins of replication, host cells are transformed with DNA controlled by appropriate expression control elements (e.g., promoter, enhancer, sequences, transcription terminators, polyadenylation sites, etc.), and a selectable marker. Following the introduction of the foreign DNA, engineered cells are then allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn are cloned and expanded into cell lines. This method can advantageously be used to engineer cell lines which express the antibody or its binding fragments.

In some instances, a number of selection systems are used, including but not limited to blasticidin, zeocin, the herpes simplex virus thymidine kinase (Wigler et al., 1977, Cell 11:223), hypoxanthine-guanine phosphoribosyltransferase (Szybalska & Szybalski, 192, Proc. Natl. Acad. Sci. USA 48:202), and adenine phosphoribosyltransferase (Lowy et al., 1980, Cell 22:817) genes are employed in tk-, hgprt- or aprt-cells, respectively. Also, antimetabolite resistance are used as the basis of selection for the following genes: dhfr, which confers resistance to methotrexate (Wigler et al., 1980, Proc. Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May 1993, TIB TECH 11(5):155-215) and hygro, which confers resistance to hygromycin (Santerre et al., 1984, Gene 30:147). Methods commonly known in the art of recombinant DNA technology which can be used are described in Ausubel et al. (eds., 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.; Colberre-Garapin et al., 1981, J. Mol. Biol. 150:1).

In some instances, the expression levels of an recombinant polypeptide are increased by vector amplification (for a review, see Bebbington and Hentschel, the use of vectors based on gene amplification for the expression of cloned genes in mammalian cells in DNA cloning, Vol. 3. (Academic Press, New York, 1987)). When a marker in the vector system expressing a recombinant polypeptide is amplifiable, an increase in the level of inhibitor present in culture of host cell will increase the number of copies of the marker gene. Since the amplified region is associated with the nucleotide sequence of the recombinant polypeptide, production of the recombinant polypeptide will also increase (Crouse et al., 1983, Mol. Cell Biol. 3:257).

In some instances, any method known in the art for purification of an recombinant polypeptide is used, for example, by chromatography (e.g., ion exchange, affinity, particularly by affinity for the specific antigen after Protein A, and sizing column chromatography), centrifugation, differential solubility, or by any other standard technique for the purification of proteins.

### Expression Vectors

In some embodiments, vectors include any suitable vectors derived from either a eukaryotic or prokaryotic sources. In some cases, vectors are obtained from bacteria (e.g. E. coli), insects, yeast (e.g. Pichia pastoris), algae, or mammalian sources. Exemplary bacterial vectors include pACYC177, pASK75, pBAD vector series, pBADM vector series, pET vector series, pETM vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Exemplary insect vectors include pFastBac1, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1, or pPolh-MAT2.

In some cases, yeast vectors include Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pAO815 Pichia vector, pFLD1 Pichia pastoris vector, pGAPZA, B, & C Pichia pastoris vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.

Exemplary algae vectors include pChlamy-4 vector or MCS vector.

Examples of mammalian vectors include transient expression vectors or stable expression vectors. Mammalian transient expression vectors may include pRK5, p3xFLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3xFLAG-CMV 7.1, pFLAG-CMV 20, p3xFLAG-Myc-CMV 24, pCMV-FLAG-MAT1, pCMV-FLAG-MAT2, pBICEP-CMV 3, or pBICEP-CMV 4. Mammalian stable expression vector may include pFLAG-CMV 3, p3xFLAG-CMV 9, p3xFLAG-CMV 13, pFLAG-Myc-CMV 21, p3xFLAG-Myc-CMV 25, pFLAG-CMV 4, p3xFLAG-CMV 10, p3xFLAG-CMV 14, pFLAG-Myc-CMV 22, p3xFLAG-Myc-CMV 26, pBICEP-CMV 1, or pBICEP-CMV 2.

In some instances, a cell-free system is a mixture of cytoplasmic and/or nuclear components from a cell and is used for in vitro nucleic acid synthesis. In some cases, a cell-free system utilizes either prokaryotic cell components or eukaryotic cell components. Sometimes, a nucleic acid synthesis is obtained in a cell-free system based on for example Drosophila cell, Xenopus egg, or HeLa cells. Exemplary cell-free systems include, but are not limited to, E. coli S30 Extract system, E. coli T7 S30 system, or PURExpress®.

Host Cells

In some embodiments, a host cell includes any suitable cell such as a naturally derived cell or a genetically modified cell. In some instances, a host cell is a production host cell. In some instances, a host cell is a eukaryotic cell. In other instances, a host cell is a prokaryotic cell. In some cases, a eukaryotic cell includes fungi (e.g., yeast cells), animal cell or plant cell. In some cases, a prokaryotic cell is a bacterial cell. Examples of bacterial cell include gram-positive bacteria or gram-negative bacteria. Sometimes the gram-negative bacteria is anaerobic, rod-shaped, or both.

In some instances, gram-positive bacteria include Actinobacteria, Firmicutes or Tenericutes. In some cases, gram-negative bacteria include Aquificae, Deinococcus-Thermus, Fibrobacteres-Chlorobi/Bacteroidetes (FCB group), Fusobacteria, Gemmatimonadetes, Nitrospirae, Planctomycetes-Verrucomicrobia/Chlamydiae (PVC group), Proteobacteria, Spirochaetes or Synergistetes. Other bacteria can be Acidobacteria, Chloroflexi, Chrysiogenetes, Cyanobacteria, Deferribacteres, Dictyoglomi, Thermodesulfobacteria orThermotogae. A bacterial cell can be Escherichia coli, Clostridium botulinum, or Coli bacilli.

Exemplary prokaryotic host cells include, but are not limited to, BL21, Mach1™, DH10B™, TOP10, DH5α, DH10Bac™, OmniMax™, MegaX™, DH12S™, INV120, TOP10F′, INVαF, TOP10/P3, ccdB Survival, PIR1, PIR2, Stbl2™, Stbl3™, or Stbl4™.

In some instances, animal cells include a cell from a vertebrate or from an invertebrate. In some cases, an animal cell includes a cell from a marine invertebrate, fish, insects, amphibian, reptile, or mammal. In some cases, a fungus cell includes a yeast cell, such as brewer's yeast, baker's yeast, or wine yeast.

Fungi include ascomycetes such as yeast, mold, filamentous fungi, basidiomycetes, or zygomycetes. In some instances, yeast includes Ascomycota or Basidiomycota. In some cases, Ascomycota includes Saccharomycotina (true yeasts, e.g. Saccharomyces cerevisiae (baker's yeast)) or Taphrinomycotina (e.g. Schizosaccharomycetes (fission yeasts)). In some cases, Basidiomycota includes Agaricomycotina (e.g. Tremellomycetes) or Pucciniomycotina (e.g. Microbotryomycetes).

Exemplary yeast or filamentous fungi include, for example, the genus: Saccharomyces, Schizosaccharomyces, Candida, Pichia, Hansenula, Kluyveromyces, Zygosaccharomyces, Yarrowia, Trichosporon, Rhodosporidi, Aspergillus, Fusarium, or Trichoderma. Exemplary yeast or filamentous fungi include, for example, the species: Saccharomyces cerevisiae, Schizosaccharomyces pombe, Candida utilis, Candida boidini, Candida albicans, Candida tropicalis, Candida stellatoidea, Candida glabrata, Candida krusei, Candida parapsilosis, Candida guilliermondii, Candida viswanathii, Candida lusitaniae, Rhodotorula mucilaginosa, Pichia metanolica, Pichia angusta, Pichia pastoris, Pichia anomala, Hansenula polymorpha, Kluyveromyces lactis, Zygosaccharomyces rouxii, Yarrowia lipolytica, Trichosporon pullulans, Rhodosporidium toru-Aspergillus niger, Aspergillus nidulans, Aspergillus awamori, Aspergillus oryzae, Trichoderma reesei, Yarrowia lipolytica, Brettanomyces bruxellensis, Candida stellata, Schizosaccharomyces pombe, Torulaspora delbrueckii, Zygosaccharomyces bailii, Cryptococcus neoformans, Cryptococcus gattii, or Saccharomyces boulardii.

Exemplary yeast host cells include, but are not limited to, Pichia pastoris yeast strains such as GS115, KM71H, SMD1168, SMD1168H, and X-33; and Saccharomyces cerevisiae yeast strain such as INVSc1.

In some instances, additional animal cells include cells obtained from a mollusk, arthropod, annelid or sponge. In some cases, an additional animal cell is a mammalian cell, e.g., from a primate, ape, equine, bovine, porcine, canine, feline or rodent. In some cases, a rodent includes mouse, rat, hamster, gerbil, hamster, chinchilla, fancy rat, or guinea pig.

Exemplary mammalian host cells include, but are not limited to, 293A cell line, 293FT cell line, 293F cells, 293 H cells, CHO DG44 cells, CHO-S cells, CHO-K1 cells, FUT8 KO CHOK1, ExpiCHO-S cells, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, and T-REx™-HeLa cell line.

In some instances, a mammalian host cell is a stable cell line, or a cell line that has incorporated a genetic material of interest into its own genome and has the capability to express the product of the genetic material after many generations of cell division. In some cases, a mammalian host cell is a transient cell line, or a cell line that has not incorporated a genetic material of interest into its own genome and does not have the capability to express the product of the genetic material after many generations of cell division.

Exemplary insect host cells include, but are not limited to, Drosophila S2 cells, Sf9 cells, Sf21 cells, High Five™ cells, and expresSF+® cells.

In some instances, plant cells include a cell from algae. Exemplary insect cell lines include, but are not limited to, strains from Chlamydomonas reinhardtii 137c, or Synechococcus elongatus PPC 7942.

Articles of Manufacture

In another aspect of the disclosure, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic.

The label or package insert indicates that the composition is used for treating the condition of choice. The article of manufacture in this embodiment of the disclosure may further comprise a package insert indicating that the compositions can be used to treat a particular condition.

Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### Example 1: PC-1 Characterization and Toxicity Studies

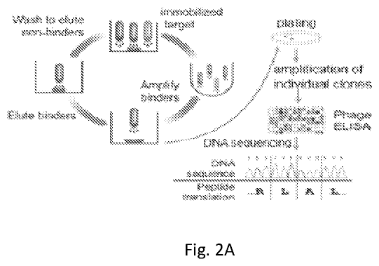

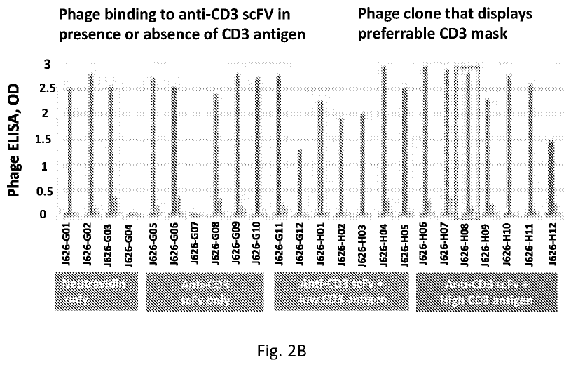

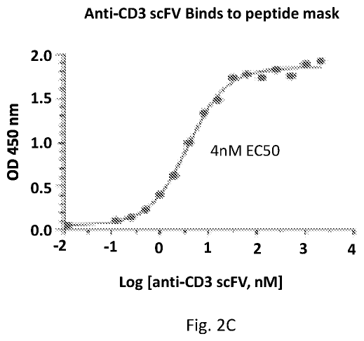

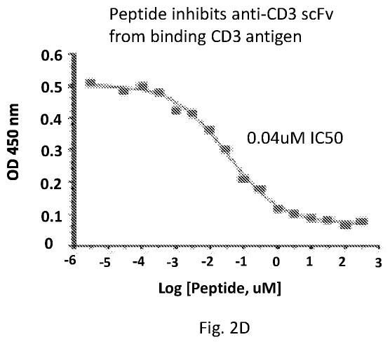

FIGS. 2A-2D illustrate mask discovery by peptide phage display. Phage displaying peptide libraries were screened for binding to surface-immobilized anti-CD3 scFv. After several bind, elute, and amplify cycles, clonal phages were screened for CD3 competitive binding by ELISA. Selected clonal phage sequences were synthesized as peptides and screened for binding and inhibition properties against anti-CD3 scFv. Peptide inhibitors were then incorporated into polypeptide complex designs.

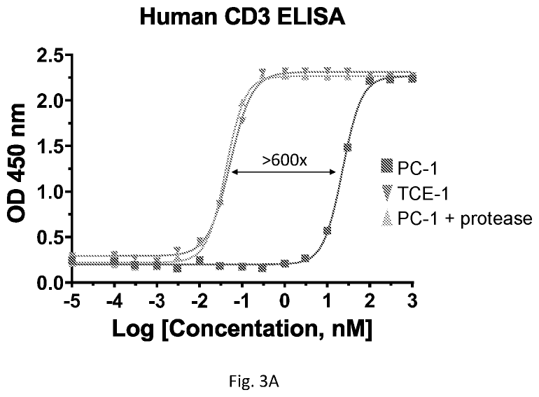

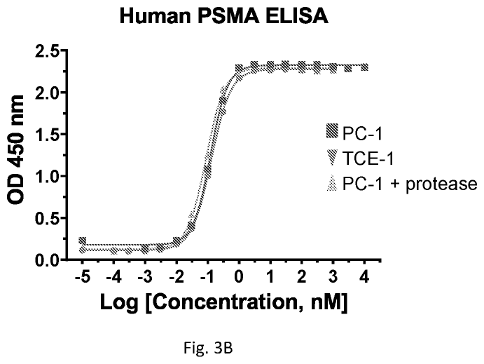

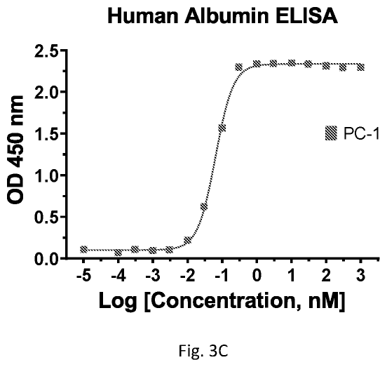

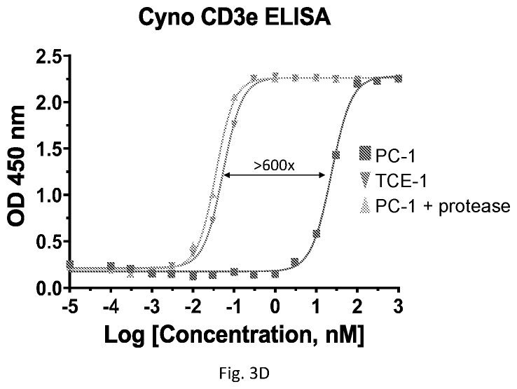

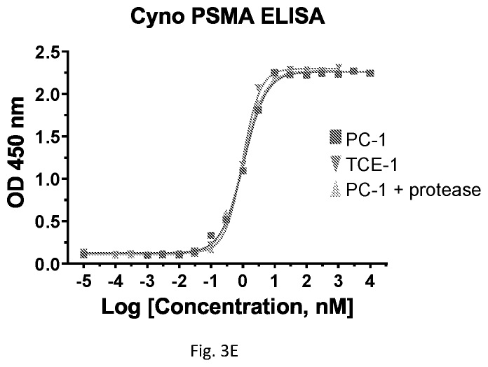

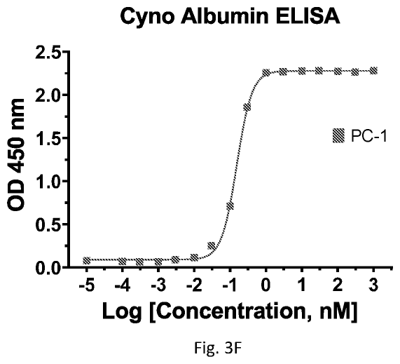

FIGS. 3A-3F illustrate binding of PC-1 to CD3 is cleavage and dose-dependent. PC-1 CD3 target engagement is cleavage dependent where masking reduces CD3 binding by >600 fold. Treatment of PC-1 with protease enzyme enables potent CD3 binding comparable to non-masked TCE-1. PC-1 exhibits potent binding to human and monkey PSMA and albumin.

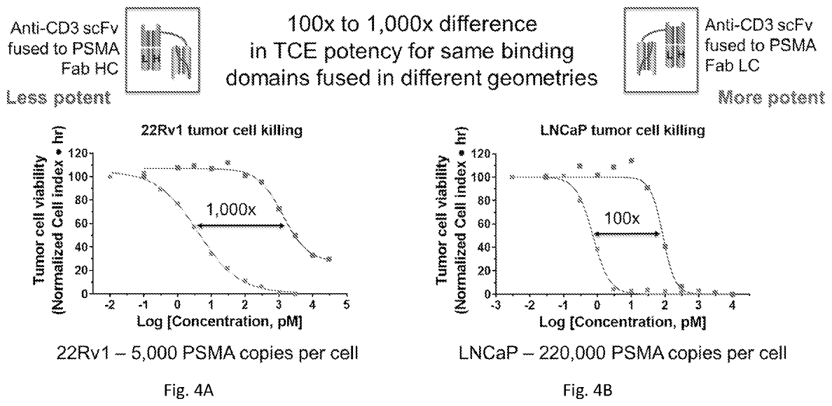

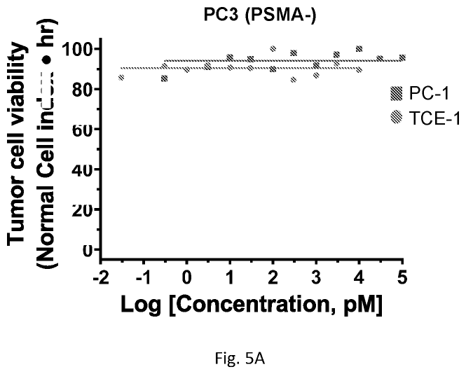

FIGS. 4A-4B illustrate TCE-1 potency depends on structure and orientation. TCE-1 activity depends on the connecting geometry of PSMA and CD3 binding domains.

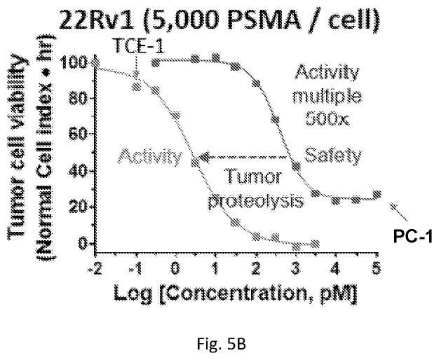

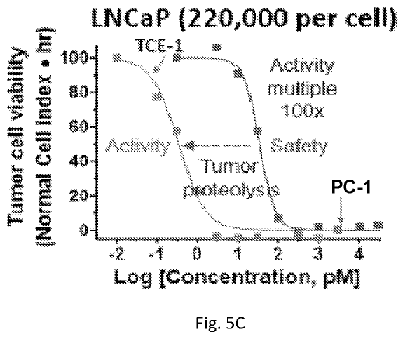

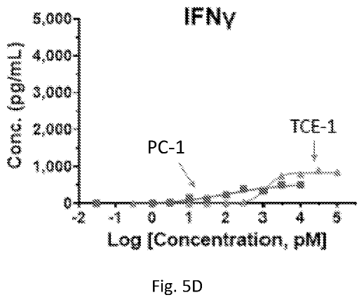

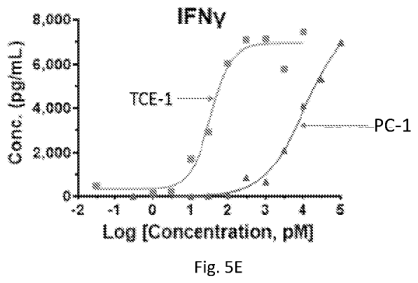

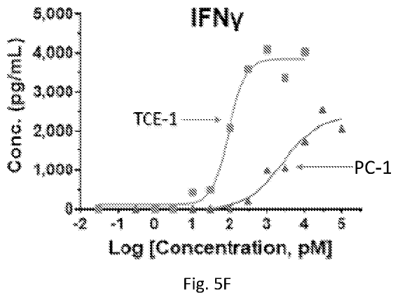

FIGS. 5A-5F illustrate activity of PC-1 is cleavage, dose- and PSMA expression-dependent. Masking of the PC-1 CD3-binding domain reduces its capacity to induce cytokine release. Functional activity in prostate cancer and T cell co-culture assays is dependent on masking and PSMA expression. PC-1 demonstrates a robust T cell functional shift that demonstrates potential for an enhanced safety profile.

Table 2 illustrates cleavable linkers feature rapid proteolysis and high serum stability. Linkers are cleaved faster than that harbored by Pacmilimab (CL-1) and maintain stability in human serum. Serum proteolytic activity is greater than blood—represents a conservative assessment of in vivo stability. The cleavable linkers are cleaved rapidly by a panel of recombinant tumor proteases which results in enhanced de-masking in the TME and anti-tumor activity. PC-1 exhibits high stability in healthy and mCRPC human donor serum with ≤1% cleavage per day. While proteolytic cleavage of PC-1 in the TME is expected to drive anti-tumor activity, a critical safety feature of PC-1 is its stability in the blood compartment, where maintenance of masking is expected to mitigate the safety risks associated with potential healthy tissue toxicity and cytokine release syndrome.

TABLE 2
Apparent Cleavage Rate of PC-1 Harboring Different Cleavable Linkers

| Tumor | CL-1 | | | | |
|---|---|---|---|---|---|
| Proteases | (ISSGLLSGRSDNH) | CL-2 | CL-3 | CL-4 | CL-5 |
| MMP2 | Y | Y | R | R | R |
| MMP7 | B | B | Y | R | R |
| MMP9 | B | B | Y | R | R |
| MMP13 | Y | Y | R | R | R |
| MMP14 | Y | Y | Y | Y | Y |
| uPa | G | G | G | G | G |
| MTSP1 | G | G | Y | Y | Y |
| Hepsin | R | R | Y | Y | Y |

Relative PC-1 serum stability cleavage rate - % per day

| | CL-1 | | | | |
|---|---|---|---|---|---|
| | (ISSGLLSGRSDNH) | CL-2 | CL-3 | CL-4 | CL-5 |
| Human | Stable; <1% | Stable; <1% | Stable; <1% | Stable; <1% | Stable; <1% |
| serum | per day | per day | per day | per day | per day |

Relative PC-1 (CL-5) serum stability in
donor serum samples (cleavage rate - % per day)

| Normal pooled | Normal pooled | |
|---|---|---|
| human serum | cyno serum | mCRPC donor serum (n = 6) |
| <1% | 6% | 1% |

Rates:
R is more than 10⁵ M⁻¹ S⁻¹;
Y is between 10⁴ M⁻¹ S⁻¹ and 10⁵ M⁻¹ S⁻¹;
G is between 2.5 × 10³ M⁻¹ S⁻¹ and 10⁴ M⁻¹ S⁻¹;
B is equal or less than 2.5 × 10³ M⁻¹ S⁻¹

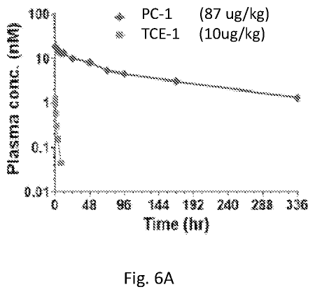

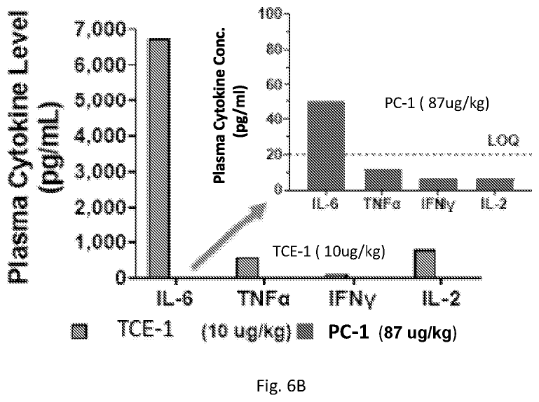

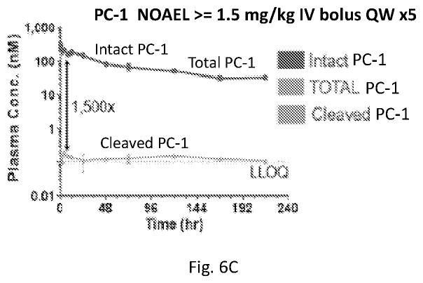

FIGS. 6A-6C and Table 3 illustrate PC-1 has extended half-life and enhanced safety profile in NHPs.

TABLE 3
Pharmacokinetics of PC-1

| PK Parameter | PC-1 | TCE |
|---|---|---|
| Dose (ug/kg) | 87 | 10 |
| Cmax (nM) | 18 | 1.7 |
| T1/2(hr) | 119 | 2 |
| Vd (L) | 0.18 | 0.2 |
| CL(ml/hr/kg) | 0.34 | 25 |

PC-1 toxicity studies support enhanced PK, safety, and drug design. PC-1 administered at 1.5 mg/kg IV bolus QW×5 in cyno monkeys achieved high exposures and long half-life without clinical signs or notable changes in clinical pathology measurements. PC-1 was highly stable in vivo with minimally detectable cleavage. Cleaved PC-1 approached LLoQ while intact (masked) PC-1 achieved 1,500× exposure multiple above its minimally detectable metabolite. Total (masked+unmasked) and intact PC-1 exhibited overlapping PK profiles further supporting PC-1 in vivo stability. Rapid clearance of TCE minimizes cleaved PC-1 accumulation and in turn minimizes risk of on-target healthy tissue toxicities and cytokine release.

PC-1 PSMA-GLP toxicity study summary in non-human primates illustrate once weekly dosing, 0.1, 0.3, 1.5 mg/kg, with a 4-week recovery. No microscopic histopathology findings were observed. Lack of TCE accumulation in vivo mitigated on-target healthy tissue toxicities and minimized cytokine release. Clinical chemistry, hematology and pathology data package support No-Observed-Adverse-effect-level (NOAEL)≥1.5 mg/kg/dose.

PC-1 exhibits enhanced safety and PK properties relative to TCE-1. The critical safety feature of PC-1 is a tumor protease-cleavable, inhibitory peptide mask, which decreases PC-1 binding to human CD3 by >600×, restricting T cell activation to the TME. In vitro, PC-1 exhibits up to 500× decrease in potency to activate T cells and induce T-cell mediated tumor cell killing relative to non-masked TCE-1. PC-1 shows an enhanced safety profile in NHPs, featuring a decrease in cytokine CRS-associated proinflammatory cytokines with NOAEL≥1.5 mg/kg/dose IV bolus QW×5. Albumin-binding domain extends the circulating half-life of PC-1 to ˜120 h in NHPs, relative to <2 h half-life of non-masked TCE, supporting the PC-1's projected once weekly clinical dosing.

Cleavage-dependent activity, half-life extended PK, potential for superior safety, and manufacturability properties of PC-1 support its further development as an attractive mCRPC therapeutic.

### Example 2: Production and Purification Process

The manufacturing process is divided into an upstream (cell culture) process, harvest and downstream (purification) process. The upstream process consists of an upstream fed-batch expression in Chinese Hamster Ovary (CHO)-K1 cell culture at a 200 L cell culture scale, through the harvest step. The downstream process uses multiple purification steps to produce a bulk drug substance. The drug substance is then subject to final formulation and addition of excipients prior to bulk filtration and fill into polycarbonate bottles.

### Summary

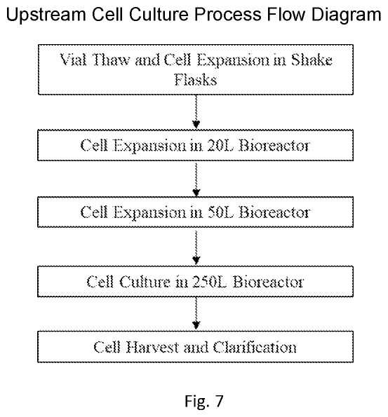

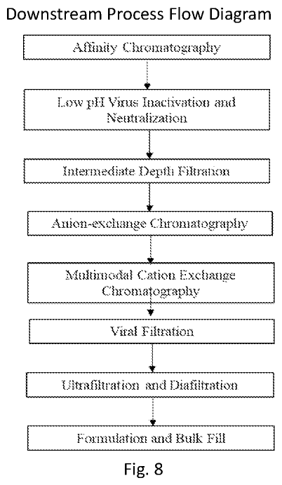

PC-1 was produced and purified following the steps shown in FIGS. 7-8. The upstream process starts with the thawing of a single vial of Master Cell Bank followed by cell culture expansion and fed-batch production of PC-1 in the harvested cell culture fluid (HCCF). The Basal Media 1 consisting of CD CHO media with glutamine, hypoxanthine and thymidine is inoculated with 1 vial of Master Cell Bank and expanded in shake flasks to a defined viable cell density of approximately (1.30-4.00)×10⁶ cells/milliliter (mL). The culture is then transferred to a 20 liter (L) disposable bioreactor filled with Basal Media 2 consisting of ActiPro media with glutamine, hypoxanthine, and thymidine, followed by culture in a 50 L single use bioreactor filled with the same media. When the viable cell density reaches an approximately viable cell density (VCD) of (3.60-6.00)×10⁶ cells/mL and viability is above 90%, the cell culture is transferred to the 250 L single-use bioreactor for fed-batch production at a 200 L cell culture process scale. The cell culture proceeds for approximately 10 days, at which point harvest is performed by depth filtration.

The downstream process starts with the capture of PC-1 from the harvested cell culture fluid (HCCF) by a Protein A affinity chromatography followed by virus inactivation at low pH, neutralization, and depth filtration steps. Host cell impurities are removed by an intermediate polishing step using anion exchange chromatography resin. PC-1 is recovered in the flow-through and it is further polished by binding to a multimodal chromatography resin with step elution. A virus-nanofiltration step is performed to remove any potential adventitious agents. The product is then subject to ultrafiltration and diafiltration with histidine buffer pH 6.3 followed by sucrose and polysorbate 20 additions to improve bulk stability. The formulated product is filtered and aseptically dispensed into single-use, sterile polycarbonate bottles and the bulk drug substance is stored at −70° C.±10° C.

### Upstream Cell Culture Process

Referring to FIG. 7, the upstream manufacturing process begins with the thaw of one vial from the Master Cell Bank. Cells are progressively expanded to achieve an appropriate cell density, using chemically defined growth medium, prior to inoculation into a 20 L disposable bioreactor unit, and subsequent 50 L and final 250 L production bioreactors. Cell density, dissolved oxygen, pH, and environmental conditions are controlled during cell expansion and culture. Upon completion of the cell culture (approximately 10 days), cells are harvested. Further details of each of the steps of FIG. 7 are provided below.

### Vial Thaw and Cell Culture in Shake Flasks

The manufacturing process is initiated when one vial from the Master Cell Bank is thawed in a 37.0° C.±1° C. water bath, and cells are aseptically transferred into a shake flask, followed by resuspension into fresh Basal Media 1 consisting of CD CHO media with glutamine, hypoxanthine and thymidine. The cell suspension is incubated at 36.5° C. with 6.0% carbon dioxide (CO₂) and a shaker speed of 120 rotations per minute (rpm) for approximately 2 days when VCD is approximately (1.10-3.00)×10⁶ cells/mL and the viability is higher than 85%. The cell culture is further sub-cultured into increasing size shake flasks with the addition of fresh Basal Media 1, at defined VCD and viability, under controlled environmental conditions. A backup inoculum expansion is generated at the N-3 stage and maintained for up to three passages.

### Cell Expansion in 20L Bioreactor

After two rounds of expansion in shake flasks and when the VCD of the cell culture reaches approximately (1.30-4.00)×10⁶ cells/mL and viability is higher than 90.0%, the cell culture is transferred to a 20 L disposable bioreactor at cell density of approximately 0.35×10⁶ cells/mL with Basal Media 2 consisting of ActiPro media with glutamine, hypoxanthine and thymidine. The culture proceeds at 36.5° C. with 6.0% CO₂ and rocker speed of approximately 20 rpm until a VCD of approximately (3.50-5.50)×10⁶ cells/mL and viability above 90%.

### Cell Expansion in 50 L Bioreactor

Cells from the 20 L bioreactor are inoculated into a 50 L single-use bioreactor filled with Basal Media 2 at a target VCD of approximately 0.40×10⁶ cells/mL and viability higher than 90%. The temperature and agitation speed are set at 36.5° C. and 140 rpm, respectively. The pH is maintained at 6.95 with CO₂ sparging and the dissolved oxygen (DO) is controlled at 40% with air/oxygen sparging. The cell culture continues to be expanded to a VCD of approximately (3.60-6.00)×10⁶ cells/mL and viability above 90%. Once this criterium is achieved, the contents are transferred into a final 250 L production bioreactor filled with Basal Media 2.

### Cell Culture in 250L Bioreactor

Cell culture is performed at the 200 L target production volume in a 250 L single-use bioreactor. After inoculation at a target VCD of approximately 1.00×10⁶ cells/mL, approximately 1.5 kilogram (kg) culture is transferred from the 250 L bioreactor to a 3 L bioreactor satellite run. The temperature is set at 36.5° C. at inoculation and shifted to 33.0° C. when VCD reaches approximately (10.00-14.00)×10⁶ cells/mL or day 5. The pH and the dissolved oxygen are monitored and controlled at approximately 6.90 and 40.0%, respectively, and the 250 L bioreactor is operated with agitation at 100 rpm and air/oxygen and CO₂ sparging. The cell culture proceeds with regular addition of feeding supplementation. The glucose concentration is tested daily, and it is maintained at approximately 6 g/L by adding 400 gram (g)/kg glucose stock solution. The culture is harvested on day 10 or when viability drops below 85%, whichever comes first.

### Cell Harvest and Clarification

When harvest criteria are met, the cell culture is harvested and clarified by depth filtration using a combination of filters to remove cells and cell debris. The depth filtration system consists of a 2-staged dual layered regenerated cellulose filters with a nominal retention rating of 6 to 30 micrometer (μm) and 0.10 to 0.85 μm, respectively. The cell culture is pumped through the filters at approximately 50 liters per square meter hour (LMH) and the initial effluent is discarded. Filtration proceeds until all of the cell culture is filtered and filters are chased with 50 millimolar (mM) Tris-acetic acid (Tris-HAc), 150 mM sodium chloride (NaCl), pH 7.4 wash buffer. The filtrate is collected into a single-use storage bag followed by 0.5/0.2 μm filtration into a single-use mixing bag. The final filtrate is mixed before proceeding to the next step.

### Downstream Purification Process

Referring to FIG. 8, the downstream process begins with the capture of PC-1 from the clarified cell culture fluid by affinity chromatography and elution at approximately pH 4.2. A virus inactivation step is then performed using a low pH process step, followed by a neutralization step and removal of any precipitate via intermediate depth filtration. Additional impurities are further removed by an intermediate polishing step using an anion exchange chromatography resin. PC-1 is recovered in the chromatographic flow-through and further polished by a bind-elute multimodal chromatography resin using a step elution scheme. A virus-nanofiltration step is performed prior to an ultrafiltration and diafiltration (UF/DF) step into histidine buffer, pH 6.3. Next, sucrose and polysorbate 20 are added at a final target concentration of 8% weight/volume (w/v) and 0.01% (w/v), respectively, to improve bulk stability. The formulated bulk is filtered and aseptically dispensed into sterile polycarbonate bottles. The drug substance is stored at −70° C.±10° C. Further details of the steps shown in FIG. 8 are provided below.

### Affinity Chromatography

The principle of this chromatography step is affinity binding using MabSelect PrismA Protein A resin to capture the target protein, PC-1, while allowing impurities to be removed by flowing through the packed column. PC-1 is then eluted from the resin using a low pH buffer. The Protein A chromatography resin is dedicated to the manufacture of PC-1 drug substance.

The column is rinsed, sanitized with 0.5 normal (N) sodium hydroxide (NaOH) and equilibrated with 50 mM Tris-HAc, 150 mM NaCl, pH 7.4 buffer. The clarified cell culture fluid is 0.5/0.2 μm filtered during loading of the column at approximately 20 to 50 g protein per liter of resin with a maximum flow rate of 300 centimeters per hour (cm/h) and minimum residence time of 5 min. Then, the column is washed with high/low salt and pH buffers to remove impurities. Bound PC-1 is eluted with 30 mM sodium acetate-acetic acid (NaAc-HAc), pH 4.2 at the same flow rate. Eluate peak collection starts at approximately 50 mAU/mm and ends at 50 milli-absorbance units per millimeter (mAU/mm) post peak. If multiple cycles are performed, the eluates are combined and mixed before proceeding to viral inactivation step.

### Low pH Virus Inactivation and Neutralization

The pH of the protein A eluate is adjusted to pH 3.6±0.1 with 1 molar (M) acetic acid (HAc) and maintained at 18-26° C., while stirring, to achieve a robust viral inactivation. After 1-2 hours at these conditions, the solution is neutralized with 1 M Tris-base to pH 5.5±0.2 and held at ambient temperature for 30-240 min before proceeding to the intermediate depth filtration step.

### Intermediate Depth Filtration

The intermediate depth filtration step removes precipitates that might have formed during the low pH virus inactivation and neutralization process steps. A regenerated cellulose depth filter with nominal 0.10 to 0.85 μm retention rating is equilibrated with 30 mM NaAc-HAc buffer before loading of the neutralized product pool at approximately 100 LMH while maintaining the filter pressure drop (dP) below 2 bar. After loading, the filters are chased with equilibration buffer and the combined filtrate is further 0.5/0.2 μm polyethersulfone (PES) filtered into a sterile mixing and storage bag before next step.

### Anion-Exchange Chromatography

The anion exchange (Capto Q) chromatography step is performed in a flow-through mode. The filtrate from the intermediate depth filtration step is adjusted to pH 6.8-7.2 and a conductivity of 3.0-8.0 millisiemens per centimeter (mS/cm) followed by a 0.5/0.2 μm PES filtration into a storage bag. An anion exchange column is sanitized with 1M NaOH and equilibrated with 50 mM Tris-HAc, 1 M NaCl, pH 7.0 followed by 50 mM Tris-HAc, pH 7.0. The loading proceeds at a maximum linear flow rate of 300 cm/h and a minimum residence time of 5 minutes and the loading capacity is maintained between 100 and 200 g/L CaptoQ resin. After loading is completed, the column is washed with 50 mM Tris-HAc, pH 7.0. The eluate is 0.5/0.2 μm PES filtered during collection which starts at approximately 50 mAU/mm during loading and ends at 50 mAU/mm post peak.

### Multimodal Cation Exchange Chromatography

The multimodal cation exchange (Capto MMC ImpRes) chromatography is used in a bind-elute mode as a polishing step to further remove impurities. Before loading, the column is rinsed, sanitized with 1M NaOH and equilibrated with 50 mM Tris-HAc, pH 7.0. The anion exchange eluate is loaded onto the column at a maximum linear flow rate of 300 cm/h and a minimum residence time of 5 minutes. The column is washed with equilibration/wash buffer until the UV₂₈₀ absorbance is below 25 mAU/mm. The wash is followed by an additional wash of 50 mM Tris-HAc, 60 mM NaCl, pH 7.0 and PC-1 is eluted from the column with a 50 mM Tris-HAc, 205 mM NaCl, pH 7.0 elution buffer at the same flow rate. Eluate collection starts at approximately ≥500 mAU/mm and ends at ≥125 mAU/mm post peak. The eluate is 0.5/0.2 μm PES filtered during collection and, if more than one cycle is performed, eluates are combined and mixed before proceeding to the viral filtration step.

### Viral Filtration

The viral filtration step removes potential viral particles and consists of a 0.5/0.2 μm pre-filter, 20 nanometer (nm) viral-retentive filter, and a 0.5/0.2 μm filter, in tandem. Prior to loading of the multimodal cation exchange eluate, the pre-filter and viral filter are equilibrated with 50 mM Tris-HAc, 205 mM NaCl pH 7.0 buffer. The maximum loading capacity of the filters is 254 liter per square meter (L/m2). During loading, the pressure differential is maintained at ≤2 bar for the pre-filter, between 0.7-1.0 bar for the viral retentive nano filter and ≤2 bar for the final 0.5/0.2 μm filter. After the loading has finished, the filters are chased with wash buffer. The final combined filtrate is mixed before proceeding with next steps.

### Ultrafiltration and Diafiltration

Ultrafiltration and diafiltration serve to adjust the in-process drug substance protein concentration and exchange buffer prior to final bulk formulation.

An Ultrafiltration/Diafiltration unit with a PES 30 kilodalton (kDa) molecular weight cut off filter cassettes is equilibrated with 50 mM Tris-HAc, 205 mM NaCl, pH 7.0 buffer until determined pH and conductivity are met. The filtrate from previous step is pumped along the membrane surface at ≤500 grams per square meter (g/m2) and is concentrated to approximately 15 grams per liter (g/L). The concentrated solution is then diafiltered with 10 mM histidine hydrochloride (His-HCl), pH 6.3 for a minimum of 6 diavolumes and until pH and conductivity criteria are met. The flux and transmembrane pressure are maintained at approximately 300 LMH and below 1.25 bar, respectively, throughout the ultrafiltration and diafiltration process. The DF pool is recovered after circulation at a low flow rate followed by a chase with 10 mM His-HCl, pH 6.3. The product is 0.5/0.2 μm filtered and the filtrate is maintained at ambient conditions for less than 3 days until the next step.

### Excipient Additions, Bulk Formulation, And Fill

During bulk formulation, sucrose and polysorbate 20 are added from stock solutions to the in-process material at a final concentration of 8% (w/v) and 0.01% (w/v) respectively, to improve product stability. This is followed by dilution with 10 mM His-HCl, pH 6.3 to the target concentration range. The formulated product is filtered through a 0.22 μm polyvinylidene difluoride (PVDF) membrane filter and approximately 4 kg of filtered product is aseptically dispensed into individual, single-use 5 L sterile polycarbonate bottles. The bulk is stored at −70° C.±10° C. All operations including additions of excipients, bulk filtration, and filling are performed in Laminar Flow Hoods.

### Example 3: Disulfide Bond Linkage Confirmation by LC-MS/MS

Disulfide bond linkages are important in protein folding and they play a significant role in both protein structure and functions. The number of disulfide bonds and their positions are important attributes for ensuring safety and efficacy of biopharmaceuticals.

In PC-1, there are 18 cysteine residues, which are cross-linked by one inter-chain disulfide bond and eight intra-chain disulfide bonds. Eight disulfide bond related peptides (DS1 to DS3 and DS5 to DS9) are expected by non-reduced Lys-C/trypsin sequential digestion. One disulfide bond related peptide (DS4) was expected by non-reduced Lys-C/trypsin/thermolysin sequential digestion. Liquid chromatography with tandem mass spectrometry (LC-MS/MS) results are provided in Table 4.

TABLE 4
LC-MS/MS Results for DS1-DS9

| | | Disulfide | | | | Theo- | | |
|---|---|---|---|---|---|---|---|---|
| SEQ | | Bond | Disulfide | Retention | | retical | PC-1 v2 | |
| ID | Peptide | Related | bond | Time | | Mass | Measured | Difference |
| NO: | No. | Sequence | linkages | (min) | PTM* | (Da) | Mass (Da) | (ppm) |
| 7 | DS1 | LSCAASGSTFYT | Chain1:C22- | 81.9 | / | 4682.2066 | 4682.2112 | 1.0 |
| | | AVMGWVR═TTL | Chain1:C96 | | | | | |
| | | YLQMNSLRPED | | | | | | |
| | | TAVYYCAAR | | | | | | |
| 8 | DS2 | GTLGLFTTADSY | Chain1:C138- | 77.1 | / | 5739.4737 | 5739.4831 | 1.6 |
| | | DYWGQGTLVT | Chain1:C148 | | | | | |
| | | VSSGGGGSGGG | | | | | | |
| | | SGGVYCGPEFD | | | | | | |
| | | ESVGCMGGGGS | | | | | | |
| | | GGGLSGR | | | | | | |
| 9 | DS3 | LSCAASGFTFNK | Chain1:C199- | 44.2 | / | 2561.1567 | 2561.1594 | 1.1 |
| | | ═TEDTAVYYCV | Chain1:C275 | | | | | |
| | | R | | | | | | |
| 10 | DS4 | LTCGSSTGAVTS | Chain1:C339- | 43.9 | / | 3896.7145 | 3896.7233 | 2.3 |
| | | GNYPNWVQQK | Chain1:C407 | | | | | |
| | | ═LSGVQPEDEA | | | | | | |
| | | EYYC | | | | | | |
| 11 | DS5a | VTITCR═FSGGG | Chain1:C454- | 76.0 | / | 5114.3590 | 5114.3616 | 0.5 |
| | | SGTDFTLTISSL | Chain1:C519 | | | | | |
| | | QPEDVATYYCQ | | | | | | |
| | | NYNSAPFTFGP | | | | | | |
| | | GTK | | | | | | |
| 12 | DS5b | LTVLGGGGSDI | | 80.3 | | 7715.6295 | 7715.6408 | 1.5 |
| | | QMTQSPSSLSAS | | | | | | |
| | | VGDRVTITCR═F | | | | | | |
| | | SGGGSGTDFTL | | | | | | |
| | | TISSLOPEDVAT | | | | | | |
| | | YYCQNYNSAPF | | | | | | |
| | | TFGPGTK | | | | | | |
| 13 | DS6 | SGTASVVCLLN | Chain1:C565- | 65.3 | / | 3555.7490 | 3555.7487 | −0.1 |
| | | NFYPR═VYACE | Chain1:C625 | | | | | |
| | | VTHQGLSSPVT | | | | | | |
| | | K | | | | | | |
| 14 | DS7a | GEC═SC | Chain1:C645- | 4.5 | / | 513.1199 | 513.1200 | 0.2 |
| | | | Chain2:C226 | | | | | |
| 15 | DS7b | SFNRGEC═SC | | 18.9 | / | 1017.3644 | 1017.3650 | 0.6 |
| 16 | DS8a | LSCAASGFAFSR | Chain2:C22- | 42.5 | / | 2474.0995 | 2474.1008 | 0.5 |
| | | ═AEDTAVYYCA | Chain2:C96 | | | | | |
| | | R | | | | | | |
| 17 | DS8b | LSCAASGFAFSR | | 55.3 | / | 3822.7552 | 3822.7596 | 1.2 |
| | | ═NTQYLQMNSL | | | | | | |
| | | RAEDTAVYYCA | | | | | | |
| | | R | | | | | | |
| 18 | DS9a | STSGGTAALGC | Chain2:C150- | 91.2 | / | 7389.6490 | 7389.6599 | 1.5 |
| | | LVK═DYFPEPVT | Chain2:C206 | | | | | |
| | | VSWNSGALTSG | | | | | | |
| | | VHTFPAVLQSS | | | | | | |
| | | GLYSLSSVVTVP | | | | | | |
| | | SSSLGTQTYICN | | | | | | |
| | | VNHK | | | | | | |
| 19 | DS9b | STSGGTAALGC | | 89.3 | / | 7916.9194 | 7916.9283 | 1.1 |
| | | LVK═DYFPEPVT | | | | | | |
| | | VSWNSGALTSG | | | | | | |
| | | VHTFPAVLQSS | | | | | | |
| | | GLYSLSSVVTVP | | | | | | |
| | | SSSLGTQTYICN | | | | | | |
| | | VNHKPSNTK | | | | | | |

*PTM = post-translational modification

### Example 4: Secondary Structure Determination by Far-UV CD

Protein far ultraviolet circular dichroism (far-UV CD) spectra can reveal the characteristic secondary structures, i.e. α-helix, β-sheet, random coil, etc. Prior to measurement, protein sample was buffer exchanged with 10 mM potassium phosphate buffer (pH 7.0) and diluted to 0.1 mg/mL. The data collection and analysis were performed with JASCO/J-815 CD spectrometer and Spectra Manager software.

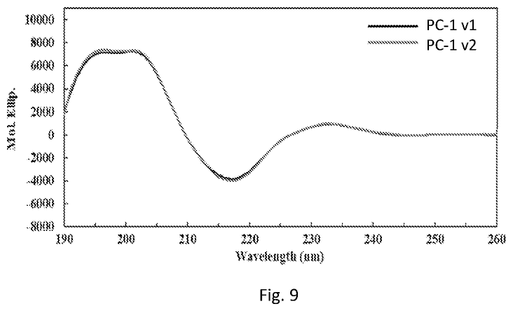

The CD spectra PC-1 in the far-UV region (190-260 nm) is shown in FIG. 9. The β-sheet and random coil were the main secondary structure composition.

### Example 5: Tertiary Structure Determination by Near UV-CD

Protein near-UV CD spectrum provides information on the protein tertiary structures. The CD spectral pattern in the 250-350 nm region is determined by the absorption, dipole orientation and the nature of the surrounding environment of the phenylalanine (250-270 nm), tyrosine (270-290 nm), and tryptophan (290-305 nm), respectively. For the measurement, the protein samples were diluted with 10 mM His, 8 (w/v) % sucrose, 0.01 (w/v) % polysorbate-20, pH 6.3 to 1.0 mg/mL. Data collection and analysis were performed by JASCO/J-815 CD spectrometer and Spectra Manager software, respectively.

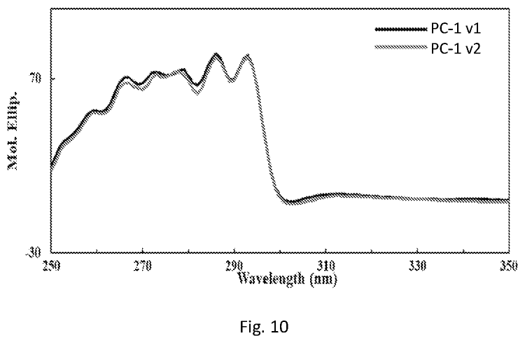

The CD spectra of PC-1 in the near-UV region (250-350 nm) is shown in FIG. 10. The Spectra similarity/structure consistency analysis was achieved by calculating the correlation coefficient of two spectra with “Quick Compare” tool of OPUS Spectroscopy Software.

### Example 6: Thermal Stability Analysis by DSC

Differential scanning calorimetry (DSC) is an experimental technique to obtain thermal transition profile of materials. It is widely used in the investigation of folding/unfolding transitions of proteins under programmed temperature increase in protein characterization. The onset (T_{Onset}) and mid-point (Tₘ) temperatures of the thermal transition (Tₘ) are commonly used as indicators of thermal stability, and the DSC thermogram reveals the thermal transition profile.

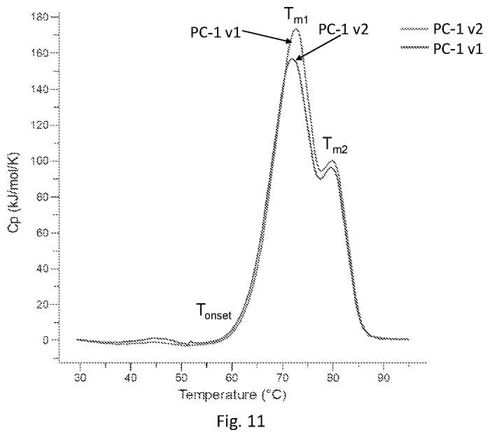

PC-1 was analyzed with MicroCal DSC from Malvern. The protein sample was diluted to 1 mg/mL with 10 mM His, 8 (w/v) % sucrose, 0.01 (w/v) % polysorbate-20, pH 6.3 before analysis. 400 microliters (μL) corresponding formulation buffer was added to a 96-well plate as the reference and 400 μL protein sample was added. The samples were heated from 10° C. to 95° C. at a heating rate of 90° C./h in the capillary DSC system. The DSC data were analyzed and fitted with MicroCal PEAQ-DSC Software 1.51. The results are shown in FIG. 11. For PC-1, the T_{Onset} was determined as 59.0° C. and the two transition mid-point temperatures (Tₘ₁ and Tₘ₂) were determined as 71.4° C. and 79.5° C.

### Example 7: Molar Mass and Size Analysis by SEC-MALS

Size exclusion chromatography coupled with multi angle light scattering (SEC-MALS) detector separates proteins based on their sizes and then measure the molecular weight of the separated components via MALS detector. The smaller proteins elute from the SEC column later while larger proteins elute at earlier retention time and results in a separation between the proteins based on their size differences. The separated components including monomer, high molecular weight species (HMWS), and low molecular weight species (LMWS) are quantified via UV detector. The absolute molar mass and size of the molecules in solution is calculated using the intensity and the angular dependence of the scattered light signal from MALS detector. The static multi-angle light scattering method characterizes the absolute molecular weight of proteins based on the principle of static light scattering, expressed in Zimm's equation. The intensity of laser scattering is directly proportional to the molecular weight and protein concentration for proteins larger than 10 nm. Therefore, those protein molecular weight can be calculated according to the relationship between scattered light intensity and angle P (θ), protein size R_{g}, as well as protein concentration c.

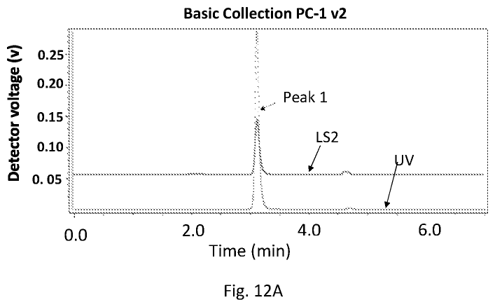

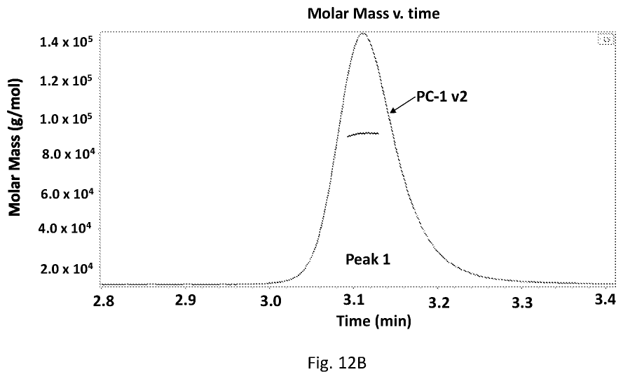

The SEC-MALS chromatograms of PC-1 are shown in FIGS. 12A-12B. The molecular weight of the main peak (peak 1) was 90.2 kDa (RT=3.111 min) which corresponds to the monomer.

### Example 8: Clinical Effects of Polypeptide Complex 1 (PC-1)

This Example illustrates the clinical effects of a polypeptide complex 1 (PC-1) disclosed herein.

PC-1 is a tumor activated T-cell engager (TRACTr) molecule designed to improve the therapeutic profile of prostate-specific membrane antigen (PSMA)-targeted T cell engagers (TCEs) in patients with metastatic castration-resistant prostate cancer (mCRPC).

### Pharmacology

Nonclinical pharmacology in vitro studies with PC-1 examined the following:
1. PC-1 binding to PSMA, cluster of differentiation 3 (CD3), and albumin antigens from mouse, rat, cynomolgus monkey, and human
2. PC-1 stability in serum of healthy human donors, mCRPC patients, and cynomolgus monkeys
3. The ability of PC-1 to induce cytokine production
4. The ability of PC-1 to induce T cell-mediated anti-tumor cytotoxic activity Results from the binding study concluded that PC-1 bound human and cynomolgus monkey PSMA, CD3, and albumin with low nanomolar affinity, while exhibiting minimal binding to mouse or rat antigens, and that CD3 binding affinity was cleavage dependent. The stability study showed that PC-1 demonstrated similar cleavage rates in healthy human serum (1%) relative to mCRPC patients' serum (2%) and that cleavage of PC-1 in cynomolgus monkey serum was 6% per day. The cytokine induction study showed that, while cleaved and non-masked versions of PC-1 exhibited dose-dependent ability to induce release of interferon γ (IFNγ), tumor necrosis factor (TNF), and interleukin (IL)-6 by immune cells, masking of PC-1 CD3-binding domain diminished its ability to induce cytokine production. This suggested that cytokine release required proteolytic PC-1 demasking. Cytokine production was not detected in the absence of target cells, suggesting that the activity of PC-1 depends on the presence of PSMA-expressing target cells. The presence of the mask also decreased the ability of PC-1 to induce T cell-mediated tumor cell killing. Additionally, the ability of PC-1 to induce T cell-mediated tumor cell killing was dose- and PSMA-dependent. The nonclinical pharmacology study examined the ability of PC-1 and PC-1-serine protease (SP) cleaved to induce cytokine release in human whole blood samples. The study examined cytokine release under 2 conditions, with soluble and wet-coated (plate-bound) test articles. No IL-2, IL-6, IL-10, TNF, and IFNγ release above the level of the untreated control (very low to no signal) was observed following stimulation with either soluble or wet-coated (plate-bound) PC-1-SP cleaved in any of the samples. No IL-2 or IFNγ cytokine release was observed in any samples following PC-1 stimulation. Following PC-1 stimulation in the plate-bound format, no IL-6, IL-10, or TNF cytokine release was observed; however, after treatment with soluble PC-1 at the highest concentrations tested (10 nM [917.4 ng/mL] and 100 nM [9174 ng/mL]), IL-6, IL-10, and TNF cytokine release was observed in a few samples. Parameters for nonclinical safety pharmacology included cardiovascular, CNS, and respiratory safety endpoints that were incorporated into a 4-week repeat-dose intravenous (IV) toxicity study in cynomolgus monkeys. There were no functional effects on the CNS or respiratory system based on clinical observations of the animals and detailed weekly examinations at doses of PC-1≤1.5 mg/kg/week. Additionally, there were no clearly defined effects from PC-1 administration on the cardiovascular systems of the animals on qualitative or quantitative electrocardiogram (ECG) parameters. Aside from a few episodes of non-test article-related arrhythmias, all ECGs were within normal limits.

### Pharmacokinetics

The pharmacokinetic (PK) properties of PC-1 were evaluated following single- and multiple-dose studies in cynomolgus monkeys. The PK of PC-1 was evaluated at 0.1 mg/kg, 0.3 mg/kg, and 1.5 mg/kg following a single IV dose to male cynomolgus monkeys. Four analytes were measured (Intact PC-1 [uncleaved PC-1], Met 1 [also referred to as PC-1-SP cleaved], Met 2 [also referred to as PC-1-MMP cleaved], and Total PC-1 [Intact (uncleaved) PC-1+Met 1+Met 2]); however, PK parameters were derived from 3 analytes (Intact PC-1, Total PC-1, and Met 1), due to Met 2 being below level of quantification (BLQ). Furthermore, clearance of the analytes was likely impacted by the presence of anti-drug antibodies (ADA), as explored further in the toxicity study below. In terms of the PK parameters, clearance of Intact and Total PC-1 remained relatively constant among the 3 dose groups. The faster clearance observed with the highest dose (1.5 mg/kg) was likely caused by the presence of ADA. The Intact PC-1 half-life (T1/2) was 111, 78.5, and 70.4 hours, at the 3 doses respectively, while the Total PC-1 T1/2 was 110, 105, and 62.1 hours, respectively. The systemic exposure as measured by area under curve (AUC0-inf) and maximum concentration (Cmax) of Intact PC-1 and Total PC-1 increased dose proportionally across all dose ranges. AUC0-last of Met 1 increased less than dose proportionally as dose levels increased from 0.1 mg/kg to 1.5 mg/kg, while Cmax increased dose proportionally across all dose ranges. The overall systemic exposure of Met 1 was minimal, and it was likely impacted by cleavage of Intact PC-1 during sample processing in the PK assay. Using the validated PK assay, sample processing induced conversion of Intact PC-1 to Met 1 of, on average, 1.87%. However, the natural cleavage in vivo is expected to be much lower. In a multi-dose toxicity study, PC-1 was administered IV at 0.1, 0.3, or 1.5 mg/kg/week to male and female monkeys, for a total of 5 administrations per animal. Minimal to no systemic exposure differences were observed between Intact PC-1 and Total PC-1.

Generally, accumulation was not observed after multiple doses, though it could not be assessed in animals with confirmed presence of ADAs. In ADA-negative animals, the median Intact PC-1 and Total PC-1 T1/2 on Day 1 and Day 22 ranged from 67.7 to 109.1 hours and 60.3 to 156.4 hours, respectively. No marked sex differences in systemic exposure as measured by Cmax and AUC0-168 h were observed on Day 1 and Day 22 at any doses in ADA-negative animals. The overall exposure of Met 1 was minimal, and it was impacted by the, on average, 1.87% cleavage of Intact PC-1 during the sample preparation step as described above. The natural in vivo cleavage is expected to be much lower. No tissue distribution studies were conducted for PC-1. No specific studies were conducted on PC-1 metabolism, as classical drug metabolic elimination does not represent an important clearance mechanism for monoclonal antibodies. Therapeutic antibodies such as PC-1 are generally catabolized into small peptides, carbohydrates, and amino acids, which are returned to the nutrient pool or excreted via the kidneys without any biological effects.

### Toxicology

The nonclinical toxicity profile of PC-1 was evaluated in a Good Laboratory Practice (GLP) 4-week, repeat-dose, IV toxicity study in cynomolgus monkeys, in in vitro and in vivo human and/or monkey cytokine release assays, and in a serum stability assay using serum from cynomolgus monkeys, healthy human donors, and patients diagnosed with mCRPC. Furthermore, immunogenicity of PC-1 was assessed in all animals during the repeat-dose toxicity study in cynomolgus monkeys. In the repeat-dose toxicity study, PC-1 was administered by IV slow bolus to male and female monkeys at doses of 0.1, 0.3, and 1.5 mg/kg/week (the control group received vehicle control) for a total of 5 administrations per animal. No changes to non-adverse hematology, clinical chemistry, and microscopic findings were observed at ≥0.1 mg/kg/week. PC-1 induced transient, dose-dependent increases in cytokines within 24 hours after the first dose at ≥0.1 mg/kg/week. Most, if not all, of the changes could be attributed to a proinflammatory response consistent with the mechanism of action of PC-1 (ie, activation of immune cell populations) and/or cytokine release observed after administration of the first dose of PC-1. Based on the lack of adverse effects noted during the dosing and recovery phases of the study, the highest non-severely toxic dose (HNSTD)/no-observed-adverse-effect level (NOAEL) was 1.5 mg/kg/week. Immunogenicity evaluation revealed that 1 out of 6 animals that received PC-10.1 mg/kg/week, 5 out of 6 animals that received 0.3 mg/kg/week, and all 10 animals that received 1.5 mg/kg/week were ADA-positive by the last dose. No animals in Group 1 (vehicle control) were found to be ADA-positive. Toxicokinetic (TK) profiles were impacted by ADA in all animals with confirmed antibodies; however, in some animals, ADAs had a lower impact on exposure after multiple doses. Furthermore, PC-1 induced dose- or concentration-dependent release of cytokines (ie IL-6, TNF, IFNγ) in vitro in the presence of tumor cells and in vivo in healthy monkeys. In vivo, cytokine release was primarily observed after the first dose and correlated with clinical signs and clinical chemistry changes indicative of cytokine release syndrome (CRS) and potential vascular leakage. PC-1 was shown to be stable in human serum with minimal cleavage in vitro. An increase in in vitro cleavage rate per day was observed in the serum from cynomolgus monkeys, but the cleavage rate was still considered to be low overall in this species.

### Phase 1 Clinical Study Design

This study is a first-in-human (FIH), Phase 1, open-label, multicenter study to assess the safety, tolerability, PK, pharmacodynamics (PD), and preliminary efficacy of PC-1 administered as a single agent in adult subjects with mCRPC. The study will be conducted in 3 parts: Dose Escalation (Part 1) with approximately 20 to 30 subjects, Cohort Backfill Expansion (Part 2) with approximately 30 subjects enrolled across three dose levels, and Dose Expansion (Part 3) with approximately 30 subjects enrolled at the recommended Phase 2 dose. Dose Escalation (Part 1) will assess the safety, tolerability, PK, PD, and preliminary efficacy of PC-1 administered by IV infusion. Cohort Backfill Expansion (Part 2) will allow for further characterization of safety and activity of dose levels. Dose Expansion (Part 3) will determine additional safety, tolerability, PK, PD, and preliminary clinical activity data with PC-1 at a dose and schedule to be determined by the Safety Review Committee after reviewing all available safety, PK, PD, and preliminary efficacy data.

Rationale for Evaluation of JANX007 in Patients with Metastatic Castration-Resistant Prostate Cancer

While TCEs are potentially a powerful class of PSMA-targeted therapies, attempts to recruit the immune system to recognize PC cells by priming them against PSMA using TCEs have thus far fallen short. Systemic activation and redirection of T cells can lead to on-target off-tumor cytotoxicity and cytokine release, resulting in severe CRS, significantly limiting dosing and ultimately efficacy (Tran et al., 2020; Bono et al., 2021). In addition, first-generation TCEs suffer from poor PK, leading to short half-lives and burdensome dosing regimens. Therefore, there is a significant unmet need to optimally leverage T cell mediated cytotoxicity in targeting tumor cells. Products that can selectively accumulate and activate within the tumor microenvironment (TME) would appear to have a significant advantage in developing a favorable risk-to-benefit profile. The PC-1-based approach is designed to offer a more focused way to activate T cells in the tumor, minimize systemic activation, and enable higher dosing, and thereby increase anti-tumor efficacy.

### Physical, Chemical, and Pharmaceutical Properties and Formulation

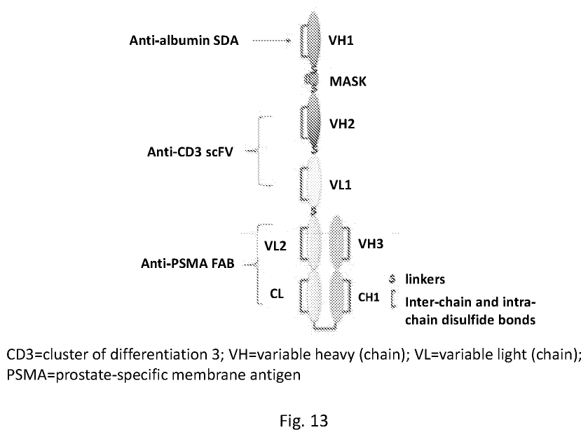

Physical and Biochemical Properties: PC-1 is a 92 kDa humanized tri-specific protein comprised of: anti-PSMA antigen-binding fragment (Fab), anti-CD3 single-chain variable fragment (scFv), and anti-albumin single domain antibody (sdAb). The molecular formula for PC-1 is C₄₀₅₀H₆₂₀₃N₁₁₀₁O₁₂₈₅S₂₇. The calculated average molecular weight of PC-1 is 91,742 Da. PC-1 TRACTr consists of two protein chains connected by a single intermolecular disulfide bond between the light chain (LC) and heavy chain (HC) of the TRACTr, and 8 intramolecular disulfide bonds. The peptide mask contains a single internal disulfide bond. The LC and HC arrangement of PC-1 TRACTr is provided in FIG. 13. The LC of the CD3 scFv is fused to the N-terminal LC of the PSMA Fab via a short flexible amino acid linker. A tandem albumin-binding sdAb, peptide mask, and tumor protease cleavable amino acid linker are fused to the amino terminus of the CD3 scFv HC. The albumin-binding sdAb is attached to the mask via a short flexible linker (FIG. 1).

### Drug Product

The PC-1 solution for injection, specifically IV infusion (referred to as PC-1 DP), is supplied as a sterile aqueous solution formulated at a nominal concentration of 2 mg/mL in 10 mM histidine, 8% (w/v) sucrose, 0.01% (w/v) polysorbate 20 (Table 5). The PC-1 drug product is contained in a 2R, Type 1 borosilicate glass vial with a 13 mm nested cap consisting of a polypropylene snap-fit cap and an embedded Flurotec® laminated elastomeric stopper plug. The formulation of the PC-1 drug product is outlined in Table 5. The physical and chemical properties of PC-1 are summarized in Table 6.

TABLE 5
Formulation Composition of PC-1 Drug Product

| | Amount | | |
|---|---|---|---|
| Component | (g) | Function | Quality Standard |
| PC-1 | 16.0 | Active | cGMP |
| | | pharmaceutical | |
| | | ingredient | |
| L-histidine | 8.312 | Buffer | Ph Eur, USP, JP |
| L-histidine | 5.541 | Buffer | Ph Eur, BP, JP |
| monohydrochloride | | | |
| monohydrate | | | |
| Sucrose | 640 | Stabilizer and | USP, NF, Ph Eur, JP, |
| | | tonicity agent | Ch.P |
| Polysorbate 20 | 0.8 | Surfactant | USP/NF, Ph Eur, JP |
| Water for injection | Q.S. | Diluent | USP |

Abbreviations:
cGMP = Current good manufacturing practice;
BP = British Pharmacopoeia;
Ch.P = Chinese Pharmacopoeia;
JP = Japanese Pharmacopoeia;
Ph Eur = European Pharmacopoeia;
Q.S. = quantity sufficient;
USP/NF = US Pharmacopeia/National Formulary

TABLE 6
Physical and Chemical Properties of JANX007 Drug Product

| Appearance (color, clarity) | Colorless 1.0 NTU | |
|---|---|---|
| Molecular weight | 91.7 | kDa |
| Melting point Tm by DSC | 72.2° | C. |
| Osmolality | 270 | mOsmol/kg |

| | |
|---|---|
| Solution pH | 6.3 |
| Isoelectric point | 9.52 |
| Potency by ELISA binding assay after | 100% relative potency |
| cleavage of CD3 mask (Relative binding | |
| when compared to the Reference Standard) | |

Abbreviations: CD3 = cluster of differentiation 3; DSC = differential scanning calorimetry; ELISA = enzyme-linked immunosorbent assay; NTU = nephelometric turbidity unit; Tm = melting temperature.

### Storage and Handling

The PC-1 DP vials are stored and shipped frozen (−20° C.).

Nonclinical Studies

Introduction: The nonclinical studies for PC-1 have been designed to support a Phase 1 clinical program in subjects diagnosed with mCRPC. Nonclinical pharmacology studies were performed in in vitro and ex vivo model systems. In vitro studies examined the following: PC-1 binding to PSMA, CD3, and albumin antigens from mouse, rat, cynomolgus monkey, and human PC-1 stability in serum of healthy human donors, mCRPC patients, and cynomolgus monkeys; The ability of PC-1 to induce cytokine production; The ability of PC-1 to induce T cell mediated anti-tumor cytotoxic activity

An ex vivo study examined the ability of PC-1 to induce cytokine release in human whole blood samples from human donors. The studies are described in further detail in the following sections. In vivo studies included a nonclinical single-dose PK study in cynomolgus monkeys. Nonclinical PK data provided the rationale for the proposed once-weekly dosing scheduling in the Phase 1 study. One GLP repeat-dose study evaluated the safety, tolerability and associated systemic exposure of PC-1 over 4 weeks of once weekly dosing (5 total administrations per animal) in cynomolgus monkeys, and assessed the reversibility of any effects following a 4-week recovery period.

### Nonclinical Pharmacology

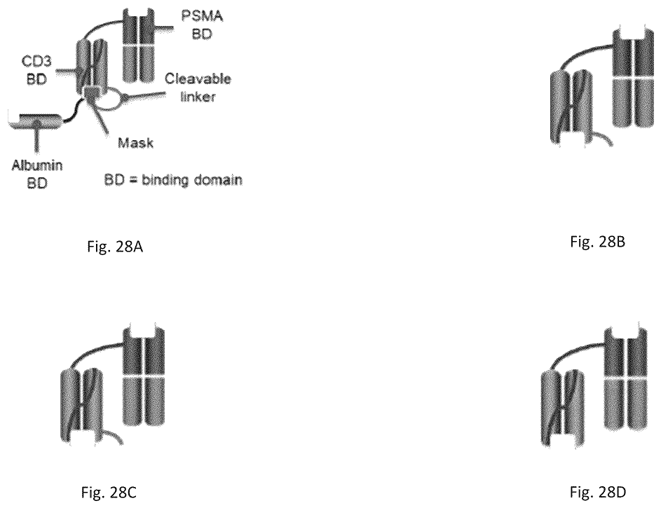

The studies were carried out with PC-1 (FIG. 28A) (uncleaved or Intact PC-1), as well as cleaved metabolites, PC-1-SP cleaved (FIG. 28B) (referred to as Met 1 in PK studies) and PC-1-MMP cleaved (FIG. 28C) (referred to as Met 2 in PK studies), and the active, non-masked molecule PC-1-TCE (FIG. 28D) (Table 7). Total PC-1 (uncleaved PC-1+Met 1+Met 2) was also measured in the PK studies. PC-1-SP cleaved and PC-1-MMP cleaved used in the in vitro pharmacology studies were derived from PC-1 by enzymatic treatment with recombinant human matriptase (MTSP1) and recombinant human matrix metalloprotease 9 (MMP9), respectively.

TABLE 7
Summary Description of PC-1 and Related Molecules

| Name | Description |
|---|---|
| PC-1 | Intact tri-specific TRACTr |
| PC-1-MMP cleaved | PC-1 that has been treated with active |
| | human recombinant MMP9 enzyme |
| PC-1-SP cleaved | PC-1 that has been treated with active |
| | human recombinant MTSP1 enzyme |
| PC-1-TCE | Recombinantly produced |
| | PSMA × CD3 bispecific protein |

Abbreviations: BD = binding domain; CD3 = cluster of differentiation 3; MMP9 = matrix metalloprotease 9; MTSP1 = matriptase; PSMA = prostate-specific membrane antigen; SP = serine protease; TCE = T-cell engager; TRACTr = tumor activated T cell engager.

### In Vitro Primary Pharmacodynamics

PC-1 Binding Affinity for Prostate-Specific Membrane Antigen, CD3, and Albumin (PC-1-001-TD): The affinity of PC-1 for mouse, rat, cynomolgus money, and human PSMA, CD3, or albumin protein was determined by standard enzyme-linked immunosorbent assay (ELISA) and was compared with the corresponding binding affinities of PC-1-SP cleaved, PC-1-MMP cleaved, and PC-1-TCE. Briefly, serial dilutions of PC-1, PC-1-SP cleaved, PC-1-MMP cleaved, or PC-1-TCE were added to plates containing immobilized mouse, rat, cynomolgus monkey, or human antigens. Bound molecules were then detected using an anti-human horseradish peroxidase-conjugated secondary antibody recognizing the PSMA-binding fragment. The resulting dose-response curves were fit to a 4-parameter variable slope regression from which the half-maximal effective concentrations (EC50s) were determined and reported as affinity (Table 8 through Table 11).

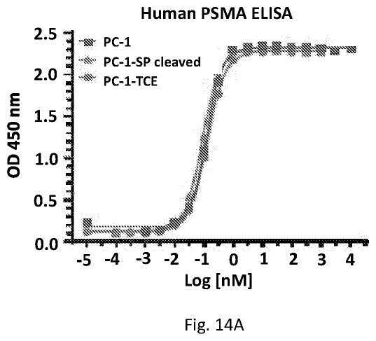

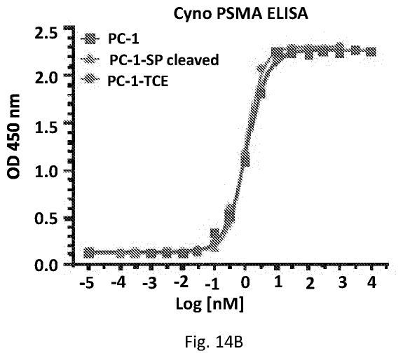

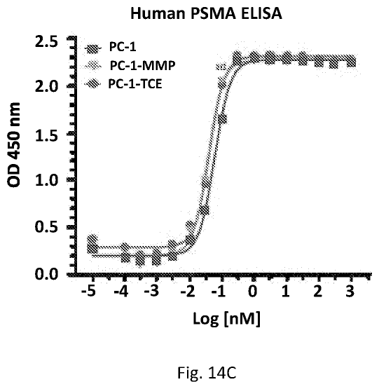

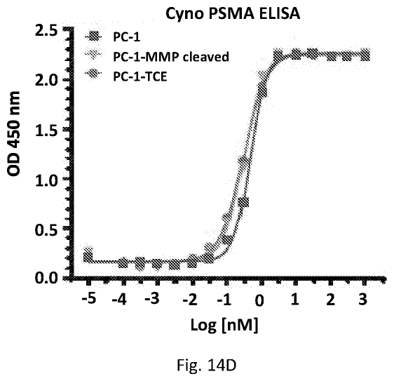

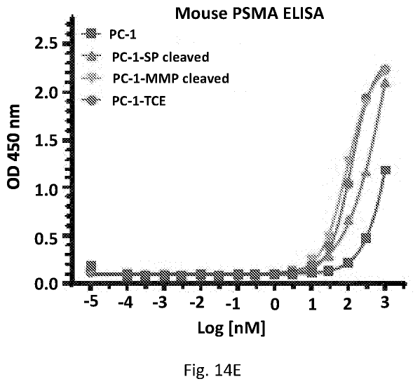

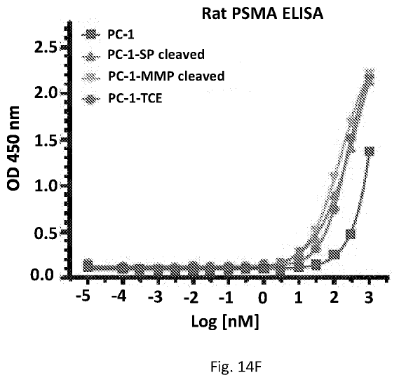

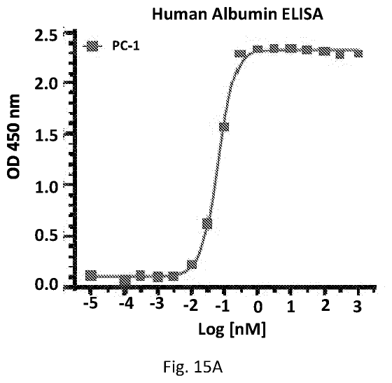

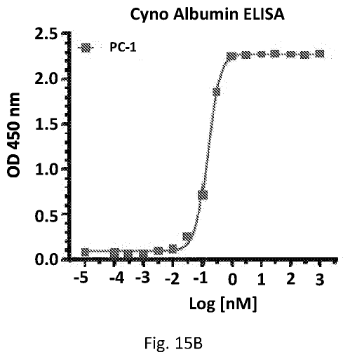

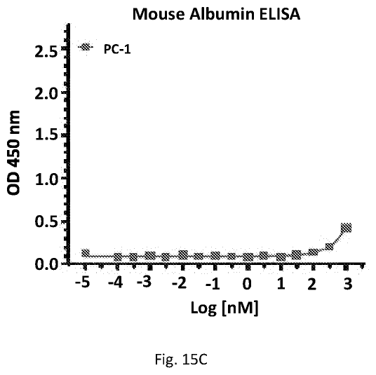

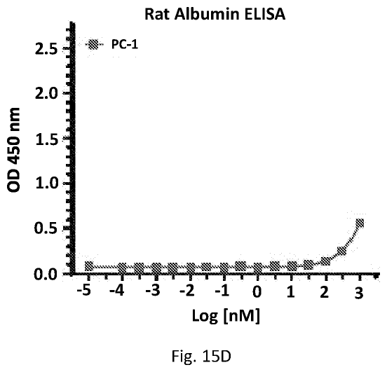

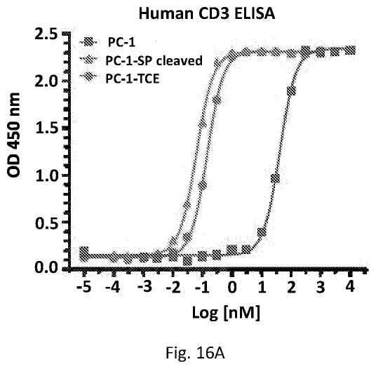

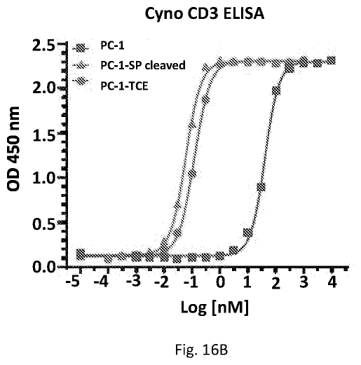

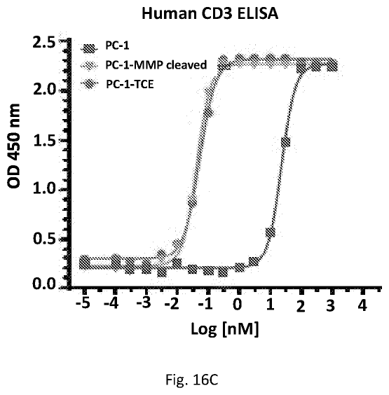

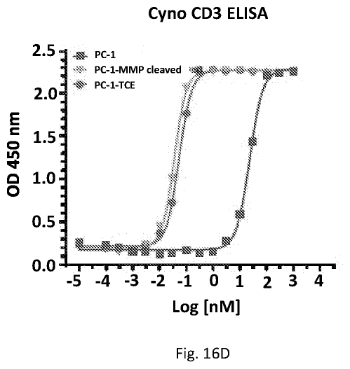

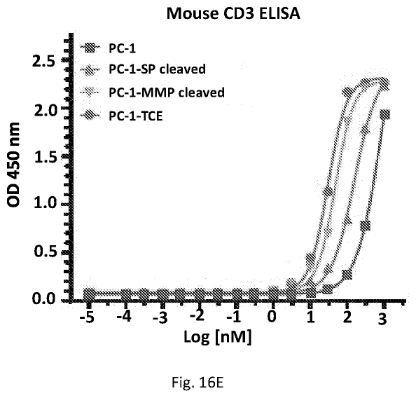

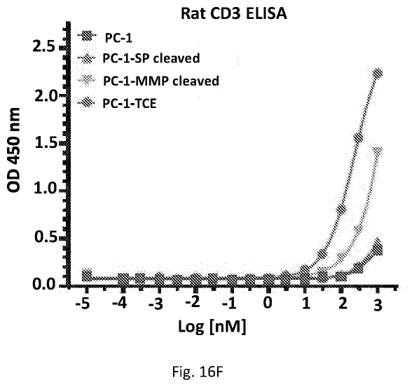

All test agents (PC-1, PC-1-SP cleaved, PC-1-MMP cleaved, and PC-1-TCE) bound to human and cynomolgus PSMA (FIGS. 14A-14F) and albumin (FIGS. 15A-15D) with low nanomolar affinity. PC-1-SP cleaved, PC-1-MMP cleaved, and PC-1-TCE exhibited low nanomolar binding to CD3. In contrast, PC-1 exhibited orders of magnitude weaker binding to CD3 due to inhibition by the peptide mask (FIGS. 16A-16F). All test agents exhibited minimal binding to respective mouse and rat antigens (FIG. 14A through FIG. 16F). The observed differences in binding of the test articles to mouse and rat antigens relative to human and cynomolgus antigens are in line with the lower percentage in sequence homology of rodent antigens relative to human and cynomolgus monkey antigens.

TABLE 8
Binding Affinity of PC-1 for PSMA, CD3, and Albumin

| | Human | Cynomolgus | Mouse | Rat |
|---|---|---|---|---|
| PC-1 | antigens | monkey antigens | antigens | antigens |
| PSMA EC50 (nM) | 0.2 | 1.1 | >100 | >100 |
| CD3 EC50 (nM) | 49 | 50 | >30 | >100 |
| Albumin EC50 (nM) | 0.1 | 0.2 | >100 | >100 |

Abbreviations: CD3 = cluster of differentiation 3; EC50 = half-maximal effective concentration; PSMA = prostate-specific membrane antigen.

TABLE 9
Binding Affinity of PC-1-SP cleaved
for PSMA, CD3, and Albumin

| PC-1-SP cleaved | Human | Cynomolgus | Mouse | Rat |
|---|---|---|---|---|
| (MTSP1 treated) | antigens | monkey antigens | antigens | antigens |
| PSMA EC50 (nM) | 0.11 | 0.97 | >100 | >100 |
| CD3 EC50 (nM) | 0.06 | 0.06 | >30 | >100 |
| Albumin EC50 (nM) | NA | NA | NA | NA |

Abbreviations:
CD3 = cluster of differentiation 3;
EC50 = half-maximal effective concentration;
MTSP1 = matriptase;
NA = not available;
PSMA = prostate-specific membrane antigen

TABLE 10
Binding Affinity of PC-1-MMP cleaved
for PSMA, CD3, and Albumin

| PC-1-MMP Cleaved | Human | Cynomolgus | Mouse | Rat |
|---|---|---|---|---|
| (MMP9 treated) | antigens | monkey antigens | antigens | antigens |
| PSMA EC50 (nM) | 0.05 | 0.42 | >100 | >100 |
| CD3 EC50 (nM) | 0.04 | 0.04 | >30 | >100 |
| Albumin EC50 (nM) | NA | NA | NA | NA |

Abbreviations: CD3 = cluster of differentiation 3; EC50 = half-maximal effective concentration; MMP9 = matrix metalloprotease 9; NA = not available; PSMA = prostate-specific membrane antigen.

TABLE 11
Binding Affinity of PC-1-TCE for PSMA, CD3, and Albumin

| | Human | Cynomolgus | Mouse | Rat |
|---|---|---|---|---|
| PC-1-TCE | antigens | monkey antigens | antigens | antigens |
| PSMA EC50 (nM) | 0.17 | 0.97 | >100 | >100 |
| CD3 EC50 (nM) | 0.05 | 0.05 | >30 | >100 |
| Albumin EC50 (nM) | NA | NA | NA | NA |

Abbreviations: CD3 = cluster of differentiation 3; EC50 = half-maximal effective concentration; NA = not available; PSMA = prostate-specific membrane antigen TCE = T cell engager.

In summary, PC-1 binds human and cynomolgus monkey PSMA and albumin with low nanomolar affinity. PC-1 CD3 binding affinity is cleavage dependent. Treatment of PC-1 with MTSP1 or MMP9 to form PC-1-SP cleaved or PC-1-MMP cleaved, respectively, results in potent CD3 binding similar to that observed for PC-1-TCE. PC-1 exhibits minimal binding to mouse or rat PSMA, CD3, and albumin. The observed differences in binding of the test articles to mouse and rat relative to human and cynomolgus PSMA, CD3, and albumin are in line with the lower percentage in sequence homology of rodent antigens relative to human and cynomolgus antigens.

Stability of PC-1 in Serum from Cynomolgus Monkeys, Healthy

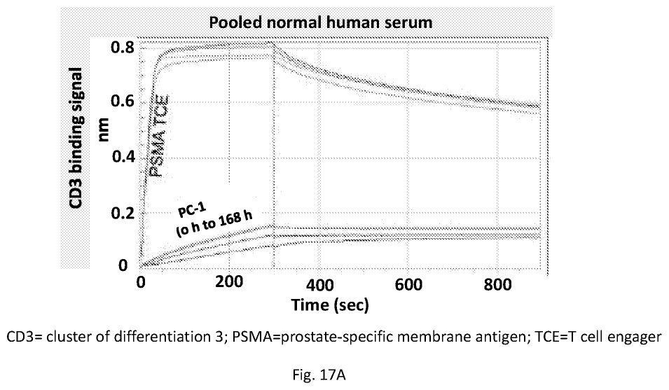

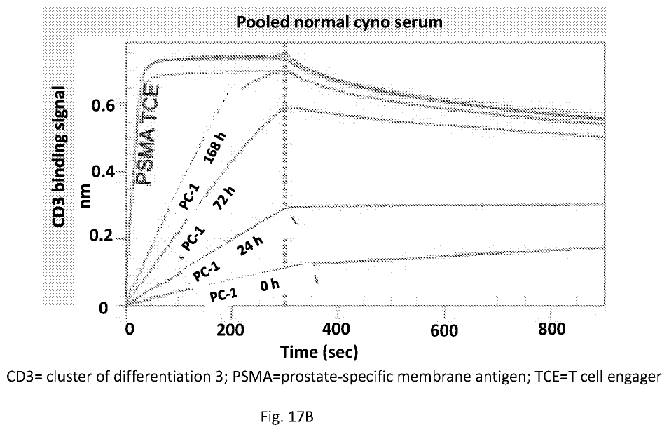

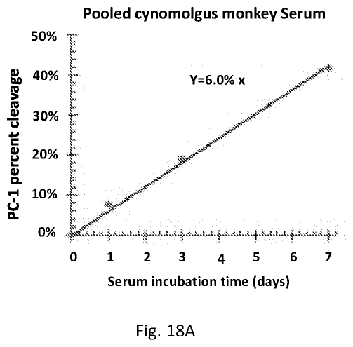

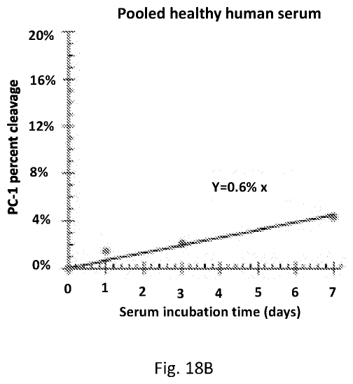

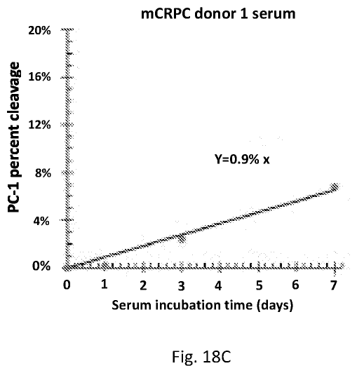

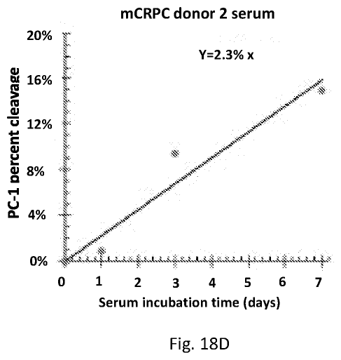

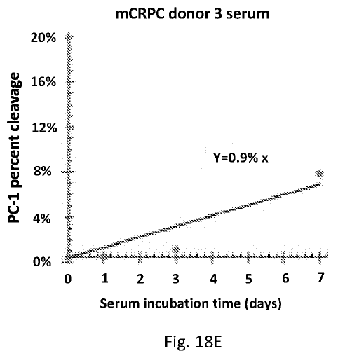

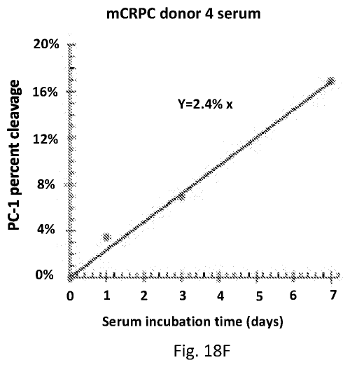

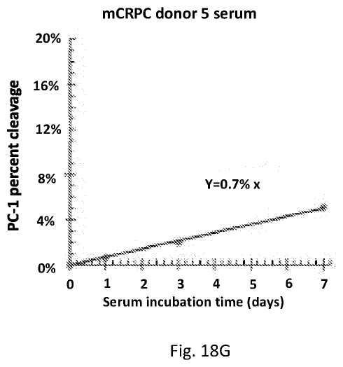

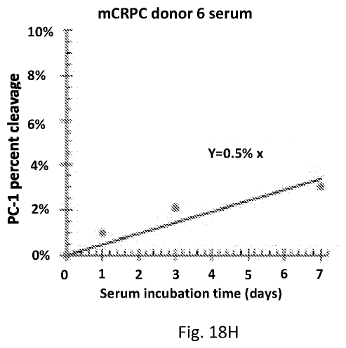

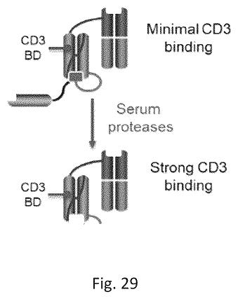

Human Donors, and Metastatic Castration-Resistant Prostate Cancer Patients (PC-1-002-TD): PC-1 stability in serum from cynomolgus monkeys, healthy human donors, and humans diagnosed with mCRPC was evaluated using kinetic binding assays with an Octet RED instrument (Octet). The Octet is an instrument used to measure the concentration of proteins and other biomolecules, measure kinetics and affinity, and screen protein-protein and protein-small molecule interactions. As illustrated in FIG. 29, serum proteases cleaves masked PC-1 with minimal CD3 binding to yield active, non-masked PC-1-TCE with strong CD3 binding. The relative concentration of cleaved PC-1 was calculated at various time points based on the initial slope of a binding curve against a CD3-coated sensor. Relative concentrations of cleaved PC-1 over time were used to calculate the cleavage rate in serum as an indicator of PC-1 stability compared with the non-masked PC-1-TCE reference sample. The resulting data are reported as percent cleavage per day (Table 12). An example of CD3 kinetic binding curve is shown in FIGS. 17A-17B. Linear regressions of percent cleavage vs time are shown in FIGS. 18A-18H. PC-1 was stable in pooled healthy human serum and mCRPC human serum measured through 168 hours. Less than 1% cleavage per day was observed in pooled healthy human serum, and 2% or less cleavage per day was observed in mCRPC serum. Somewhat higher cleavage rate of 6% per day was observed in the serum from healthy cynomolgus monkeys.

TABLE 12
Serum Stability of PC-1

| Matrix | PC-1% cleavage/day |
|---|---|
| Normal pooled human serum | <1% |
| Normal pooled cynomolgus monkey serum | 6% |
| mCRPC donor 1 serum | <1% |
| mCRPC donor 2 serum | 2% |
| mCRPC donor 3 serum | <1% |
| mCRPC donor 4 serum | 2% |
| mCRPC donor 5 serum | <1% |
| mCRPC donor 6 serum | <1% |

Abbreviations: BD = binding domain; CD3 = cluster of differentiation 3; mCRPC = metastatic castration resistant prostate cancer.

In summary, PC-1 exhibited similar cleavage rates in healthy human serum relative to the serum from mCRPC patients, with <1% and ≤52% cleavage per day, respectively. The cleavage of PC-1 in cynomolgus monkey serum was 6% per day.

PC-1-Induced Cytokine Production by Peripheral Blood Mononuclear Cells Cocultured with Target Tumor Cells Expressing Prostate-Specific Membrane Antigen

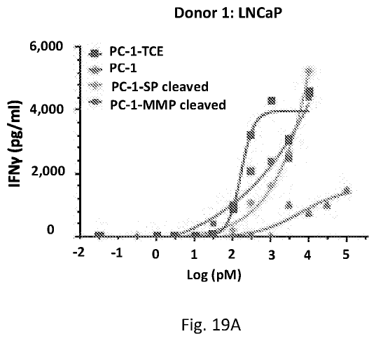

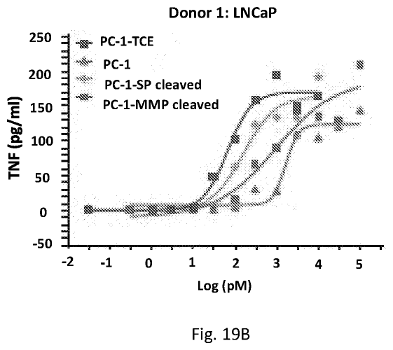

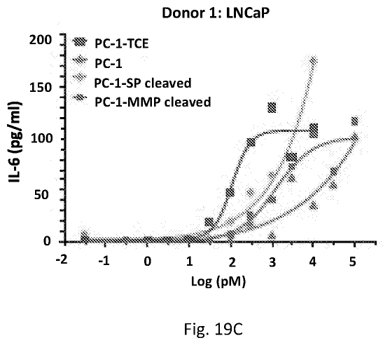

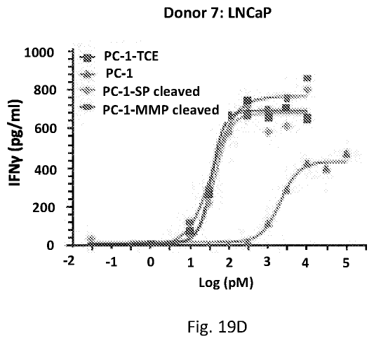

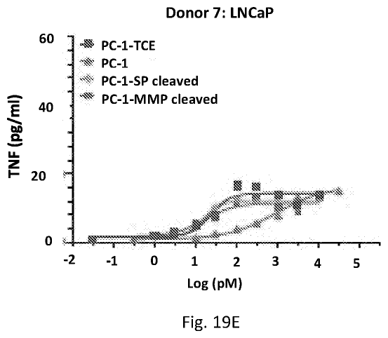

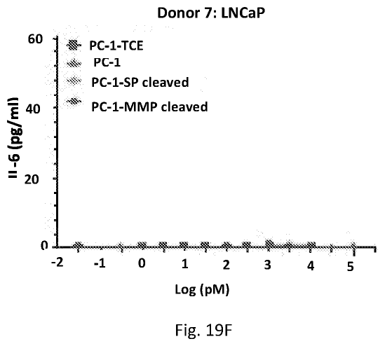

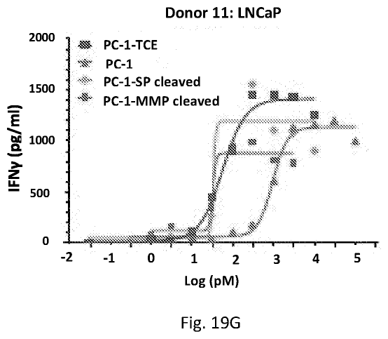

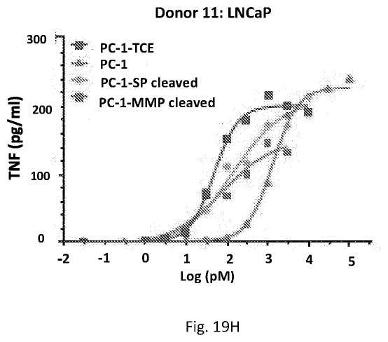

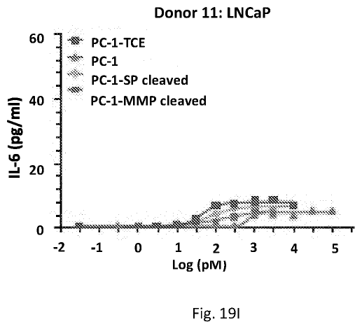

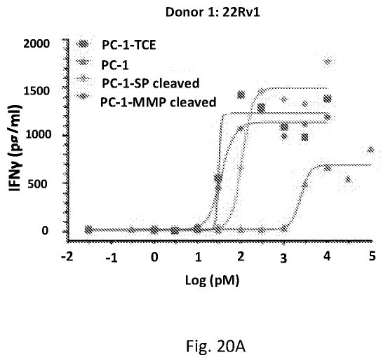

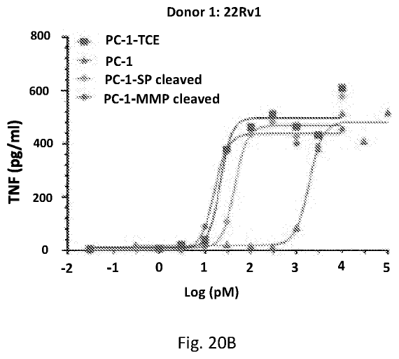

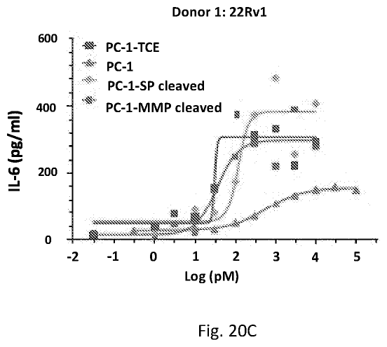

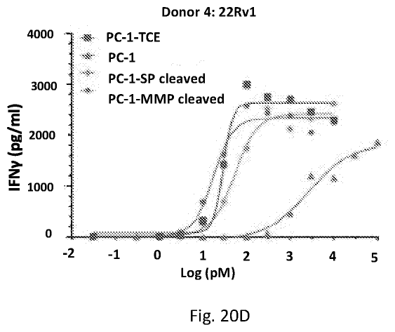

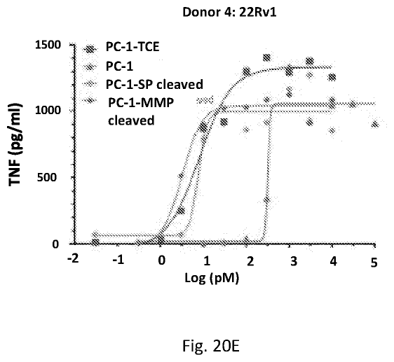

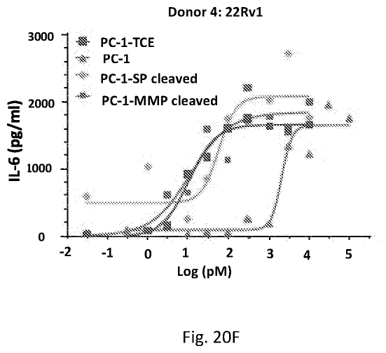

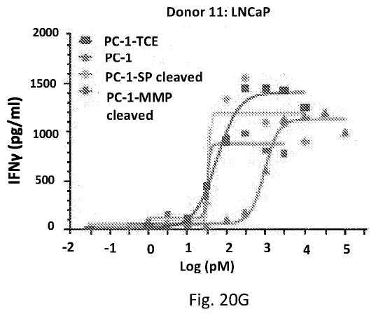

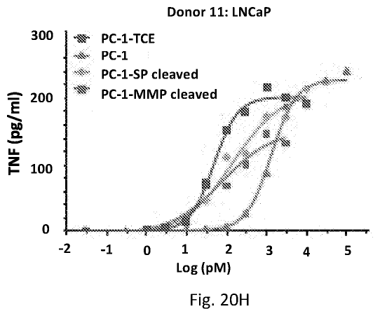

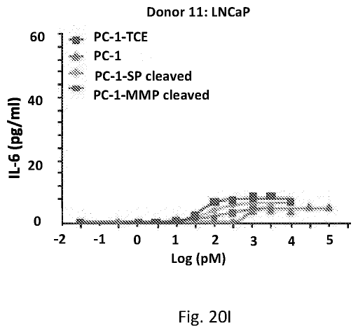

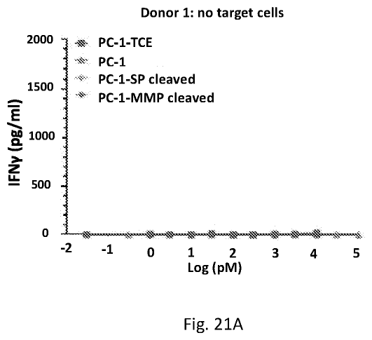

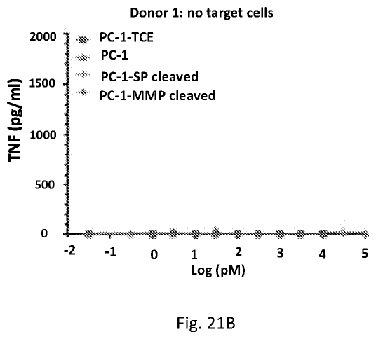

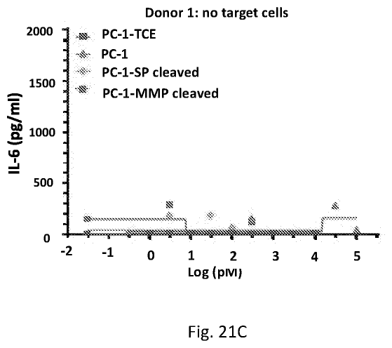

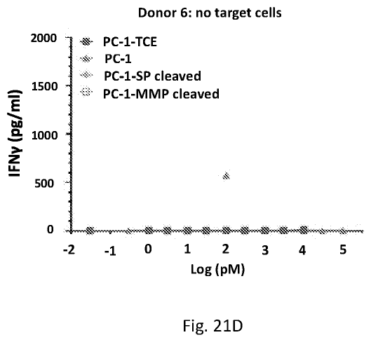

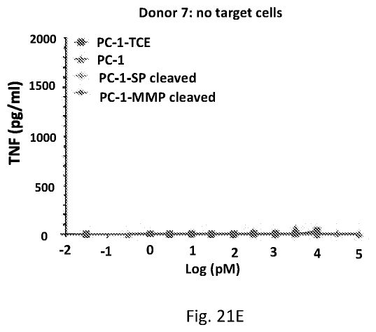

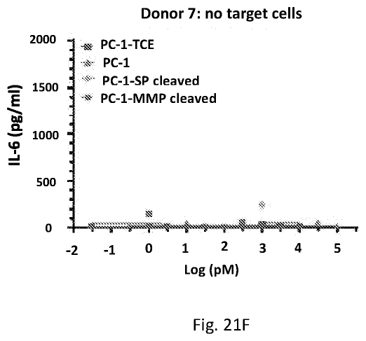

The ability of PC-1 to induce cytokine production was assessed in the coculture of peripheral blood mononuclear cells (PBMCs) and tumor cells treated with PC-1. PBMCs isolated from the blood from eight consenting healthy human donors were cultured for 72 hours with or without human PC cell lines expressing high (LNCaP), moderate (22Rv1), and minimally detectable (PC3) levels of PSMA in the presence of increasing concentrations of test articles (Dang et al., 2021; Gorges et al., 2016). Cleaved (PC-1-SP cleaved and PC-1-MMP cleaved) and non-masked (PC-1-TCE) test articles induced a potent, dose-dependent production of IFNγ, TNF, and IL-6 while masked PC-1 showed a lower ability to induce cytokine production, as illustrated by higher EC50 for all three evaluated cytokines (FIGS. 19A-19I: Each data point represents mean of two duplicate values. Generated non-linear regression response curves were used to extrapolate EC50 values (concentration of test article required to induce cytokine levels corresponding to 50% of the maximum level). Abbreviations: IFN=interferon; IL=interleukin; MMP=matric metalloprotease; PBMCs=peripheral blood mononuclear cells; SP=serine protease; TCE=T cell engager; TNF=tumor necrosis factor; FIGS. 20A-20I: Each data point represents the mean of two duplicate values. Generated non-linear regression response curves were used to extrapolate EC50 values (concentration of test article required to induce cytokine levels corresponding to 50% of the maximum level). Abbreviations: IFN=interferon; IL=interleukin; MMP=matric metalloprotease; PBMCs=peripheral blood mononuclear cells; SP=serine protease; TCE=T cell engager; TNF=tumor necrosis factor). Cytokine induction was not detected in the absence of PSMA-expressing target cells (FIGS. 21A-21F: Abbreviations: IFN=interferon; IL=interleukin; MMP=matrix metalloprotease; PBMCs=peripheral blood mononuclear cells; SP=serine protease; TCE=T cell engager; TNF=tumor necrosis factor).

In summary, masking of the CD3-binding domain of PC-1 diminished its ability to induce cytokine production, suggesting that cytokine release required proteolytic PC-1 demasking. Cleaved (PC-1-SP cleaved, PC-1-MMP cleaved) and non-masked (PC-1-TCE) versions of PC-1 exhibited a dose-dependent ability to induce cytokine production, while PC-1 had higher EC50 for all evaluated cytokines. Cytokine production was not detected in the absence of target cells, suggesting that the activity of PC-1 depends on the presence of PSMA.

### PC-1-Induced In Vitro T Cell-Mediated Killing of Target Tumor Cells Expressing Prostate-Specific Membrane Antigen

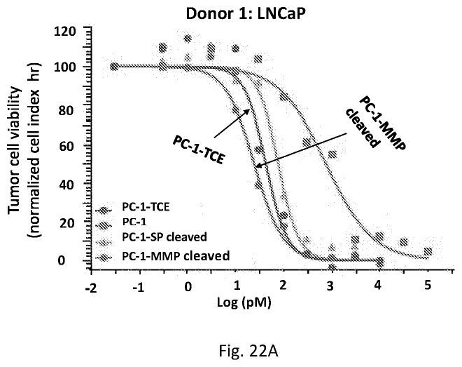

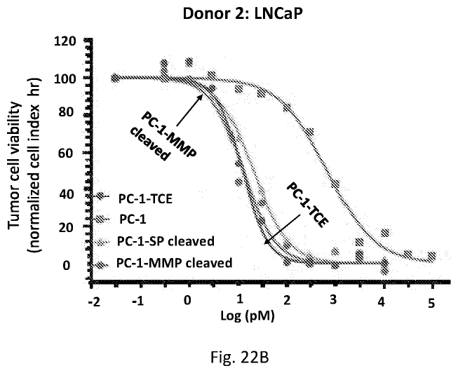

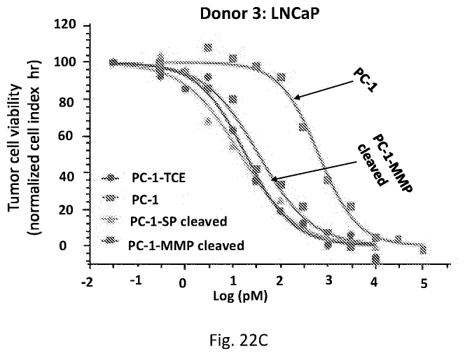

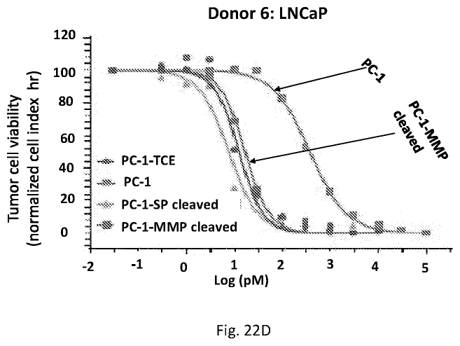

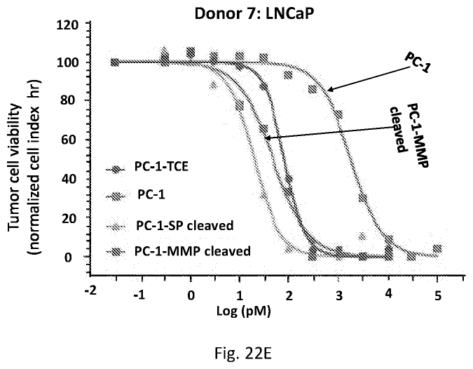

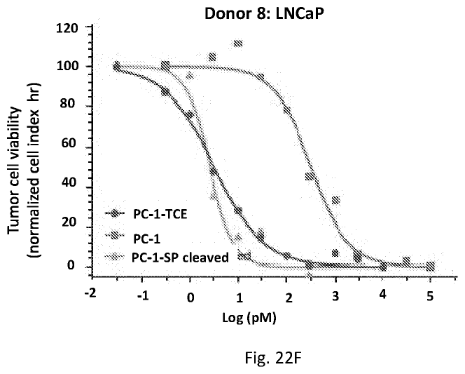

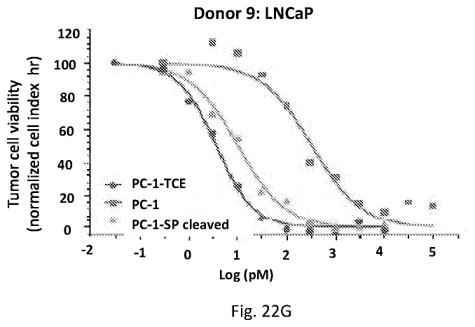

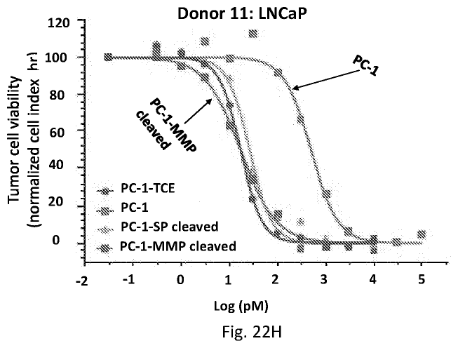

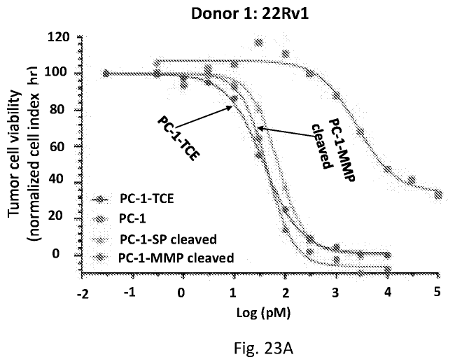

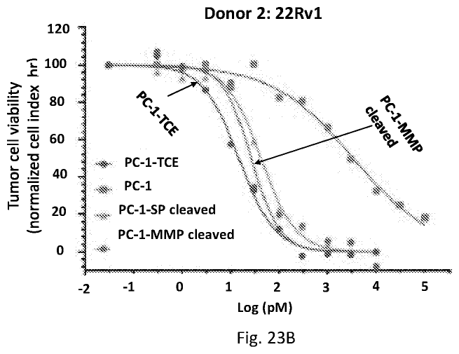

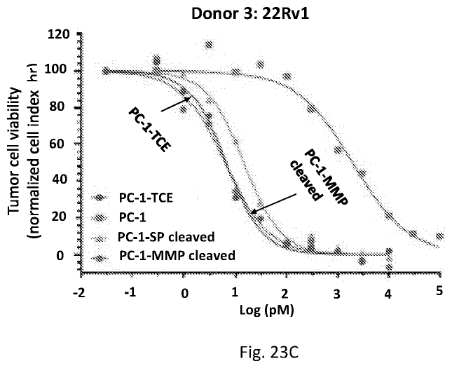

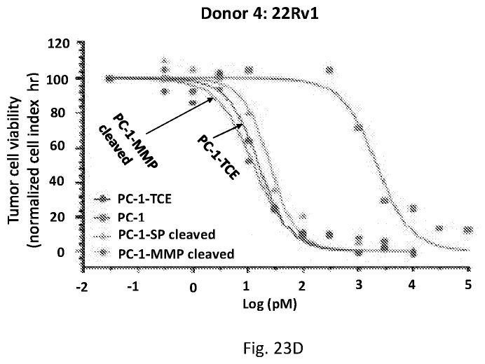

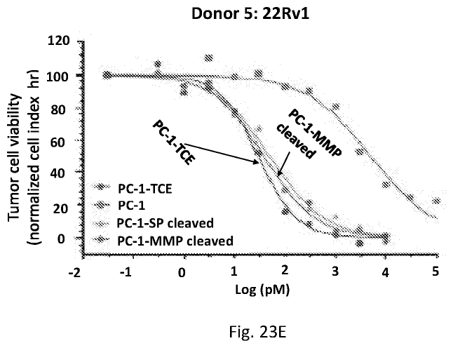

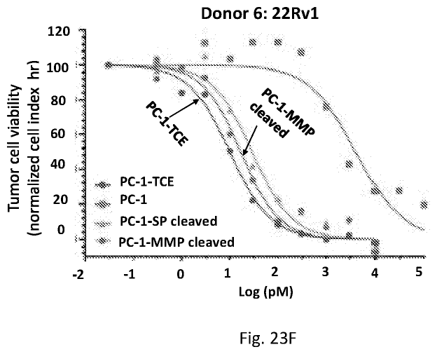

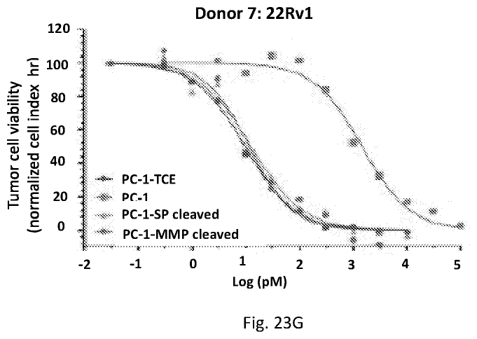

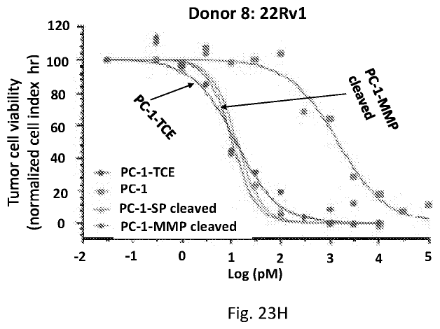

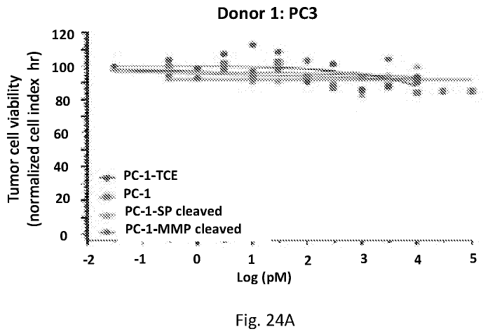

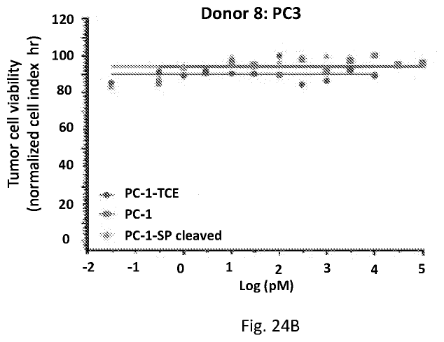

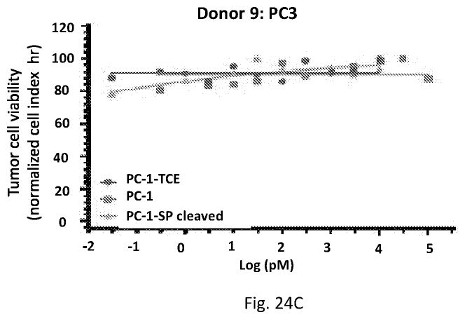

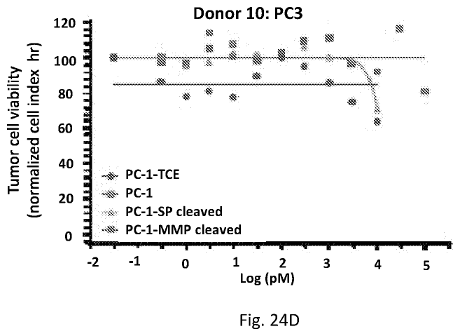

The ability of PC-1 to induce T cell-mediated target cell killing was evaluated using PBMCs derived from the blood of eight consenting healthy human donors cocultured with prostate tumor cell lines expressing high (LNCaP), moderate (22Rv1), or minimally detectable (PC3) PSMA levels in the presence of increasing concentrations of test articles. Results show that PC-1-induced T cell-mediated tumor cell killing that was dose-dependent and decreased in the presence of the mask (FIGS. 22A-22H: Each symbol represents the mean value of appropriate treatment replicates. Abbreviation: MMP=matrix metalloprotease; PBMCs=peripheral blood mononuclear cells SP=serine protease; TCE=T cell engager; and FIGS. 23A-23H: Each symbol represents the mean value of appropriate treatment replicates. Abbreviation: MMP=matrix metalloprotease; PBMCs=peripheral blood mononuclear cells; SP=serine protease; TCE=T cell engager). Relative to PC-1-SP cleaved, PC-1-MMP cleaved, and PC-1-TCE, PC-1 exhibited up to 30 times and 160 times lower ability to induce cytotoxic activity of T cells in the presence of LNCaP and 22Rv1 cells, respectively. Moreover, the magnitude of induced tumor cell killing was dependent on the level of PSMA expression, as none of the test articles induced PBMC-mediated killing of PC3 cells (representative plots shown in FIGS. 24A-24D: Each symbol represents the mean value of appropriate treatment replicates. Abbreviation: MMP=matric metalloprotease; PBMCs=peripheral blood mononuclear cells; SP=serine protease; TCE=T cell engager.)

In summary, the masking of the CD3-binding domain diminishes the ability of PC-1 to induce T cell-mediated killing of target tumor cells, with up to 160 times lower cytotoxic activity relative to the cleaved (PC-1-SP cleaved and PC-1-MMP cleaved) and non-masked (PC-1-TCE) test articles. The ability of PC-1 to induce T cell-mediated tumor cell killing is dose- and cleavage-dependent and requires PSMA expression.

### Ex Vivo Primary Pharmacodynamics

Cytometric Bead Array Analysis for Human Cytokines Using Whole Blood from Healthy Human Donors

The ability of PC-1 and PC-1-SP cleaved to induce cytokine release was assessed in whole blood samples derived from 10 healthy human donors using a cytometric bead array approach. The stimulation was done for 24 hours in soluble and wet-coated (plate-bound) formats. Stimulation with staphylococcal enterotoxin B (SEB) in soluble format, positive control, resulted in cytokine production in samples from all donors, demonstrating that these cells have the capacity to release cytokines in response to a stimulus. No cytokine release was observed for any samples in the absence of treatment. No cytokine release was observed when stimulation was done in wet-coated (plate-bound) format. No release of IL-2, IL-6, IL-10, TNF, and IFNγ above the level of the untreated control (low to not detected) was detected in any of the samples following stimulation with PC-1-SP cleaved in either format. No IL-2 or IFNγ release was observed following PC-1 stimulation for all donors in either format. No IL-6, IL-10, or TNF cytokine release was observed following PC-1 stimulation in the plate-bound format. After treatment with soluble PC-1 at the highest concentrations tested (10 nM [917.4 ng/mL] and 100 nM [9174 ng/mL]), IL-6, IL-10, and TNF release was observed in a few samples.

### Safety Pharmacology

Standalone safety pharmacology studies were not conducted with PC-1 per the ICH S6(R1) and ICH S9 guidelines. Cardiovascular, CNS and respiratory safety pharmacology endpoints were incorporated into a 4-week, repeat-dose, IV toxicity study in cynomolgus monkeys.

No clearly defined effects of PC-1 administration were observed on qualitative or quantitative ECG parameters. Sinus tachycardia, defined as an average heart rate greater than 270 beats per minute, occurred at 4 instances in 3 animals administered 1.5 mg/kg/week post-dose on Day 1 vs the control group where one instance was observed in 1 animal post-dose. Sinus tachycardia is often a normal variant in cynomolgus monkeys. While most post-dose instances occurred following the high dose, there were no significant effects of PC-1 administration on the group mean heart rate. Therefore, the tachycardia was likely not PC-1-related. One animal that received 1.5 mg/kg/week had three atrial premature complexes (APCs) on the Day 1 ECG. Infrequent APCs have been reported to occur sporadically in naïve cynomolgus monkeys, and as APCs were observed in a single animal, the arrhythmia was not considered test article-related. No PC-1-related respiration rate or body temperature changes were noted during the 4-week repeat-dose toxicity study. There were no PC-1-related CNS changes based on daily clinical signs and weekly detailed clinical observations during the study.

### Pharmacokinetics and Product Metabolism in Animals

Pharmacokinetics in Cynomolgus Monkeys: The PK properties of PC-1 were evaluated following a single-dose (non-GLP) PK study and repeat-dose GLP toxicity study in cynomolgus monkeys. Determination of analytes, Intact PC-1 (also referred to as uncleaved PC-1), Total PC-1 (PC-1+Met 1+Met 2), and the metabolites Met 1 (PC-1-SP cleaved) and Met 2 (PC-1-MMP cleaved) in non-human primate K2EDTA plasma was performed.

### Single-Dose PK Study

In the single-dose PK study, the PK of PC-1 was evaluated at the dose levels of 0.1 mg/kg, 0.3 mg/kg, and 1.5 mg/kg administered to male cynomolgus monkeys. Four analytes (Intact PC-1, Total PC-1, Met 1, and Met 2) were measured; however, PK parameters were derived from 3 analytes (Intact PC-1, Total PC-1, and Met 1). Met 2 was excluded from the TK parameter calculation because the concentrations were BLQ for most samples. Furthermore, the clearance of the analytes was likely impacted by the presence of ADAs. The Intact and Total PC-1 clearance remained relatively constant among the 3 dose groups. The faster clearance observed with the highest dose (1.5 mg/kg) was likely caused by the presence of ADAs. The mean half-life (T1/2) of Intact PC-1 was 111, 78.5, and 70.4 hours, respectively, for the 3 dose groups, while the Total PC-1 T1/2 was 110, 105, and 62.1 hours, respectively. The systemic exposure, as measured by AUC0-inf and Cmax, of Intact PC-1 and Total PC-1 increased with increasing dose across all dose ranges. The Met 1 AUC0-last increased less than dose proportionally as the dose levels increased from 0.1 mg/kg to 0.3 mg/kg and 0.1 mg/kg to 1.5 mg/kg, while Cmax increased dose proportionally. The systemic exposure as measured by AUC0-last and Cmax of Met 1 increased dose proportionally as dose levels increased from 0.3 mg/kg to 1.5 mg/kg. The overall exposure of Met 1 was minimal, and the cleavage of Intact PC-1 during sample processing for the PK assay likely impacted Met 1 generation. Using the validated PK assay, it was demonstrated that sample processing induced conversion of Intact PC-1 to Met 1 of, on average, 1.87%. Therefore, the natural cleavage in vivo is expected to be much lower. This study also analyzed cytokine production in cynomolgus monkey serum by flow cytometry. In general, cytokine elevations were transient and dose-dependent. In animals administered ≥0.1 mg/kg of PC-1, levels of IL-6 were elevated at 8 hours post-dose and decreased to pretest levels by 48 hours post-dose. In animals administered ≥0.3 mg/kg, levels of IL-2 were variably elevated at 2 hours post-dose and decreased to baseline by 24 hours post-dose. An increase in IL-5, IL-10, TNF, and IFNγ was only observed in animals administered 1.5 mg/kg (highest dose) of PC-1, and these values returned to baseline 24 to 48 hours post-dose.

### Repeat-Dose GLP Toxicity Study

In this GLP toxicity study, male and female cynomolgus monkeys received the vehicle control (Group 1) or once-weekly IV injection of PC-1 at 0.1 mg/kg/dose (Group 2), 0.3 mg/kg/dose (Group 3), or 1.5 mg/kg/dose (Group 4), for a total of 5 administrations per animal. In the immunogenicity study, ADAs were detected in a number of animals in each dose group (summarized in Section 4.4.1). ADAs had an impact on the TK profile of the test articles, as discussed below. Overall, minimal to no systemic exposure differences were observed between Intact PC-1 and Total PC-1. Accumulation was not observed after multiple doses but could not be assessed fully in Group 3 in males and in any animals in Group 4 due to the presence of ADA. No marked accumulation was observed at 0.1 mg/kg/week (Group 2) in both sexes and females at 0.3 mg/kg/week (Group 3) that were ADA-negative. In ADA-negative animals, the median Intact PC-1 T1/2 on Day 1 and Day 22 ranged from 67.7 to 109.1 hours across all of the doses tested. In ADA-negative animals, the median Total PC-1 T1/2 on Day 1 and Day 22 ranged from 60.3 to 156.4 hours across all of the doses tested. No marked differences in systemic exposure between the sexes, as measured by Cmax and AUC_{0-168 h}, were observed at 0.1 mg/kg/week on Days 1 and 22. With 0.3 and 1.5 mg/kg/week, no marked differences between the sexes were observed on Day 1; however, due to the presence of ADA and its impact on the Total PC-1 TK profile, Day 22 exposures could not be compared between the sexes. The overall exposure of Met 1 was minimal, and this was impacted by the, on average, 1.87% cleavage of Intact PC-1 during sample preparation for the TK assay. The natural cleavage is expected to be much lower. No exposure to any of the analytes was observed in any Group 1 animals. Immunogenicity was assessed in all animals. No animals from Group 1 (vehicle control) were found to be ADA-positive, while 1 of 6 animals from Group 2 (0.1 mg/kg/week), 5 of 6 animals from Group 3 (0.3 mg/kg/week), and all 10 animals from Group 4 (1.5 mg/kg/week) were confirmed to be ADA-positive by the last dose. TK profiles were impacted in all of the animals that were ADA-positive; however, ADA had lower impact on exposure after multiple doses in some animals. Two of the ADA-positive female animals in Group 3 still attained substantial exposure after the Day 22 dose with approximate doubling of AUC0-168 h (2.0-fold increase) and Cmax (1.9-fold increase) compared to the Day 1 dose. In addition, two ADA-positive female animals in Group 4 also had substantial exposure as measured by AUC0-168 h and Cmax after the Day 22 dose, with AUC0-168 h values approximately 54% and Cmax values approximately 96% of the respective values observed after the Day 1 dose.

### Metabolism

No specific studies were conducted on PC-1 metabolism, as classical drug metabolic elimination does not represent an important clearance mechanism for monoclonal antibodies. Antibodies such as PC-1 are generally catabolized into small peptides, carbohydrates, and amino acids, which are returned to the nutrient pool or excreted via the kidneys without any biological effects (Wang et al., 2008). Thus, in accordance with the ICH S6(R1) guidance, no metabolism studies have been conducted for PC-1.

### Excretion

Renal elimination is relatively unimportant for monoclonal antibodies, as their large size limits the extent of their glomerular filtration. Thus, in accordance with the ICH S6(R1) guidance, no excretion studies have been conducted for PC-1.

### Pharmacokinetic Drug Interactions

No pharmacokinetic drug interaction studies have been performed for PC-1. In general, antibodies such as PC-1 are not metabolized by cytochrome P450 (CYP) enzymes or transported by P glycoprotein (Pgp) or related adenosine triphosphate-binding cassette membrane transporters. Cytokines produced by activated lymphocytes may impact the levels of Pgp and the activity of CYP enzymes (Harvey and Morgan, 2014). The clinical relevance of PC-1 immune modulation and potential cytokine production that could impact Pgp and CYP is unknown, but a clinically relevant drug-drug interaction effect is considered highly unlikely (Seitz and Zhou, 2007; Huang et al., 2010).

### Toxicology

Repeat-Dose Toxicity Studies: PC-1 was evaluated in a GLP, 4-week, once-weekly, repeat-dose, toxicity study in cynomolgus monkeys (Table 13). Consistent with the intended clinical route of administration, the toxicity study was conducted using the IV route of administration. The cynomolgus monkey was selected as the pharmacologically relevant species because of high PSMA, CD3, and albumin protein sequence homology with human antigens, and nearly equivalent binding of PC-1 to target antigens (PSMA, CD3, and albumin) in monkeys and humans. In addition, PC-1 was also evaluated for cytokine release in vitro and in vivo and for serum stability.

TABLE 13
Overview of the PC-1 Toxicology Program

| | | | |
|---|---|---|---|
| | | Concentrations | GLP |
| Study | Study Number | or Doses | Compliance |

Repeat-Dose Toxicity

| | | | |
|---|---|---|---|
| 4-Week IV Toxicity | B49-0027-TX | 0, 0.1, 0.3, | GLP |
| in Monkeys with a | | 1.5 mg/kg/week | |
| 4-Week Recovery | | | |

Abbrevitaion: GLP = Good Laboratory Practice.

PC-1 was administered by IV bolus to male and female monkeys (3/sex/group) at doses of 0 (vehicle control), 0.1, 0.3, or 1.5 mg/kg/week (5 doses total). Additional animals (2/sex/group) at 0 and 1.5 mg/kg/week were assessed after a 4-week recovery period for the reversibility of any PC-1-related effects. The dosing regimen was selected based on the half-life of PC-1 in cynomolgus monkeys, and was designed to have a similar or more intensive dosing regimen than the clinical dosing regimen. No PC-1-related mortality, food consumption or body weight changes, body temperature, respiration rate, coagulation, urinalysis, or macroscopic findings were observed during the dosing or recovery phases. No PC-1-related ophthalmology and electrocardiographic findings were observed.

### Clinical Signs

Cold to touch and decreased activity were observed for 1 male at 1.5 mg/kg/week after the first dose of PC-1; the clinical signs gradually resolved before the second dose. The clinical signs were likely related to the cytokine induction observed after the first dose. During the dosing or recovery phases, no other clinical signs were noted in any other animals.

### Hematology

PC-1-related non-adverse hematology changes included erythroid changes consistent with decreased erythropoiesis and hemolysis (decreased red blood cell [RBC] mass, regenerative erythroid response, mean corpuscular hemoglobin [MCH], and mean corpuscular hemoglobin concentration [MCHC]) and increased leukocyte and platelet counts consistent with a pro-inflammatory response at ≥0.1 mg/kg/week. Decreases in RBC mass partially reversed, changes in reticulocyte counts fully reversed, and MCH and MCHC changes showed limited to no reversibility at the end of a 4-week recovery period in animals administered 1.5 mg/kg/week. Non-adverse, minimally to markedly decreased lymphocyte counts in animals administered ≥0.1 mg/kg/week correlated with immunophenotyping findings of decreased mean absolute number of total T cells, cytotoxic T cells, helper T cells, and natural killer (NK) cells at 24 hours post-dose in animals administered ≥0.1 mg/kg/week and were consistent with the expected pharmacology of the test article (ie, transient sequestration of activated lymphocytes). Mildly decreased monocyte counts were also noted on Day 2 in animals administered ≥0.1 mg/kg/week; however, a cause was not apparent. Minimally decreased platelet counts on Day 2 were consistent with a transient nonspecific effect of a large molecule test article on platelets in monkeys or potentially due to PC-1-related increases in proinflammatory cytokines within 24 hours following the first dose. On or after Day 15, PC-1-related leukocyte changes included minimally to mildly increased neutrophil counts in females administered ≥0.1 mg/kg/week, minimally or mildly increased lymphocyte counts in males and females administered 1.5 mg/kg/week, and moderately increased eosinophil counts in males administered 1.5 mg/kg/week and females administered ≥0.3 mg/kg/week that were consistent with a pro-inflammatory response. PC-1-related mildly or moderately increased platelet counts on or after Day 8 in animals administered ≥0.1 mg/kg/week were also consistent with a proinflammatory response. Lymphocyte, monocyte, and platelet count changes were largely reversible by the end of the 4-week recovery phase.

### Clinical Chemistry

PC-1-related non-adverse clinical chemistry findings noted on Day 2 (16-24 hours post-dose) were consistent with a transient, more pronounced proinflammatory response following the first dose administration that likely resulted in vascular leakage and/or decreased blood pressure (not measured) and correlated with increased cytokine release within 24 hours following the first dose. Mildly to markedly increased urea and creatinine on Day 2 in animals administered ≥0.3 mg/kg/week were consistent with kidney dysfunction that was potentially due to decreased kidney tissue perfusion; however, a direct effect on the kidney could not be excluded and correlative microscopic kidney findings on Day 31 were limited to minimal to moderate mononuclear cell infiltrate. Increased urea and creatinine had evidence of reversibility by Day 8. More pronounced decreases in serum albumin in animals administered ≥0.1 mg/kg/week and minimally decreased sodium and chloride on Day 2 in animals administered ≥0.3 mg/kg/week were also consistent with potential vascular leakage or potential kidney dysfunction. Less pronounced decreases in serum albumin were noted through Day 31 and had mostly reversed by the end of the 4-week recovery period. Decreased albumin at these later time points, together with minimally or mildly increased globulin, was consistent with a pro-inflammatory response. Additional PC-1-related serum chemistry changes included reversible minimally or mildly decreased total cholesterol.

### Organ Weights

PC-1-related organ weight differences on Day 31 consisted of increased spleen weights in animals administered ≥0.1 mg/kg/week and increased liver weights in animals administered 1.5 mg/kg/week. Increased spleen weights did not appear to correlate with the dose. However, the magnitude of the differences was consistent with a test article-related finding and correlated with microscopic findings of minimal or mild increased lymphocyte cellularity in the white pulp of the spleen. Increased liver weights correlated with minimal perivascular mononuclear cell infiltrates. PC-1-related organ weight differences at the end of the recovery period consisted of increased spleen weights in males administered 1.5 mg/kg/week, were consistent with partial reversibility and lacked a microscopic correlate.

### Microscopic Findings

PC-1-related non-adverse microscopic findings on Day 31 consisted of minimal to marked mononuclear cell infiltrates, which often had a perivascular pattern, in the liver, gallbladder, adrenal medulla, cervix, vagina, epididymides, seminal vesicles, and urinary bladder and increased lymphocyte cellularity in the white pulp of the spleen in animals administered ≥0.1 mg/kg/week. However, these microscopic findings often had a poor dose relationship. Microscopic findings were consistent with a pro-inflammatory response but were not associated with evidence of tissue injury and were, therefore, considered not adverse. Microscopic findings had mostly reversed by the end of the recovery period, and the remaining changes at the end of the recovery phase were of uncertain relationship to PC-1 treatment.

### T/B/NK Cell Immunophenotyping

In monkeys administered PC-1 at ≥0.1 mg/kg/week, the mean absolute number of total T cells, cytotoxic T cells, helper T cells, and NK cells decreased 24 hours post-dosing, compared with pretest. A transient B cell count increase was noted at 1.5 mg/kg/week after the first dose. PC-1 treatment-induced transient, minimal increases in the percentages of CD69+ helper T cells, CD69+ cytotoxic T cells, and Ki67+ helper T cells after the first dose. All observed changes in peripheral white blood cell (WBC) cell populations were transient and returned to levels similar to pretest values prior to the third or fifth doses.

### Cytokine Levels

PC-1 induced transient, dose-dependent increases in IL-2, IL-5, IL-6, IL-10, TNF, and IFNγ within 24 hours after the first dose at ≥0.1 mg/kg/week. Cytokine levels returned to pretest values by 24 hours after the first dose or prior to the second dose. At 1.5 mg/kg/week, the fourth dose induced a transient, minimal increase in IL-6 levels in a few animals.

### Impact of Anti-Drug Antibodies

Anti-PC-1 antibodies were detected in all groups, and incidence and titers of ADA were dose-dependent (1/6 animals at 0.1 mg/kg/week, 5/6 animals at 0.3 mg/kg/week, and 10/10 animals at 1.5 mg/kg/week). All animals in the high dose group (1.5 mg/kg/week) were ADA-positive by the end of the study, and these animals also had the highest ADA titers. Due to the presence of ADA, most animals at 1.5 mg/kg/week did not maintain exposure through the end of the dosing phase, with most animals showing a substantive decrease in exposure as assessed by the third or fourth dose. Two of the ADA-positive female animals administered 0.3 mg/kg/week still attained substantial exposure after the Day 22 dose with an approximate doubling of AUC_{0-168 h} (2.0-fold increase) and Cmax (1.9-fold increase) compared to Day 1. In addition, 2 ADA-positive female animals administered 1.5 mg/kg/week also sustained substantial exposure as measured by AUC_{0-168 h} and Cmax after the Day 22 dose, with approximately 54% of exposure vs Day 1 as measured by AUC_{0-168 h} and approximately 96% of exposure vs Day 1 as measured by Cmax. Exposure at 0.1 mg/kg/week was similar to or higher in males and females on Day 22 compared with Day 1. There were no differences in toxicity findings in females that maintained exposure at ≥0.3 mg/kg/week vs animals that did not maintain exposure. Therefore, the assessment of potential toxicity in this study at all dose levels was considered to be valid.

### Conclusions

In conclusion, male and female cynomolgus monkeys were administered vehicle control or 0.1, 0.3, or 1.5 mg/kg/week PC-1 via IV injection for 4 weeks, followed by a 4-week recovery period. Non-adverse hematology (decreased RBC mass and regenerative erythroid response; transiently decreased WBC populations followed by increases; changes in platelet counts), clinical chemistry (transiently increased urea, creatinine, globulin, and bilirubin; transiently decreased sodium and chloride, and decreased albumin and cholesterol), organ weight (increased spleen and liver weights), and microscopic findings (minimal to marked mononuclear cell infiltrates in several tissues) were observed at ≥0.1 mg/kg/week. PC-1 induced transient, dose-dependent increases in cytokines within 24 hours after the first dose at ≥0.1 mg/kg/week. Most, if not all, of the changes could be attributed to a proinflammatory response consistent with the mechanism of action of PC-1 and/or cytokine release observed after administration of the first dose of PC-1. Based on the lack of adverse effects noted during the dosing and recovery phases of the study, HNSTD/NOAEL was 1.5 mg/kg/week. Systemic exposure (Cmax and AUC_{0-168 h}) of Intact PC-1 was 43,500 ng/mL and 2,340,000 h ng/mL, respectively, after the first dose at the NOAEL, sexes combined.

### Genotoxicity, Carcinogenicity, and Reproductive Toxicity Studies

Per the ICH S6(R1) guideline, the range and type of genotoxicity studies routinely conducted for small molecule drug products are generally not applicable to biotechnology-derived products. It is not expected that a humanized tri-specific protein, such as PC-1, would interact directly with DNA or other chromosomal material. Thus, mutagenicity studies are not considered relevant and are not planned. No carcinogenicity studies have been conducted with PC-1. Given the intended patient population is subjects with mCRPC, carcinogenicity studies are not planned (per the ICH S9 guideline). Nonclinical reproductive and developmental toxicity studies have not been conducted with PC-1. Since its impact on pregnancy is unknown at this time, all subjects and their female partners will be required to practice at least 2 highly effective protocol-specified methods of birth control while participating in clinical trials of PC-1.

### Effects in Humans

Introduction: There is no clinical experience with PC-1. The current study will be the FIH Phase 1 clinical trial of PC-1. For complete patient eligibility criteria, please see the clinical study protocol. A brief summary of the planned clinical study is provided below.

### Synopsis of Phase 1 Trial Design

Study Design: This study is a FIH, Phase 1, open-label, multicenter study to assess the safety, tolerability, PK, PD, and preliminary efficacy of PC-1 administered as a single agent in adult subjects with mCRPC. The study will be conducted in 3 parts: Dose Escalation (Part 1) with approximately 20 to 30 subjects, Cohort Backfill Expansion (Part 2) with approximately 30 subjects enrolled across three dose levels, and Dose Expansion (Part 3) with approximately 30 subjects enrolled at the RP2D. Dose Escalation (Part 1) will assess the safety, tolerability, PK, PD, and preliminary efficacy of PC-1 administered by IV infusion. Dose escalation decisions will be made based on the recommendations of the Safety Review Committee, based on the review of safety data and available PK, PD, and preliminary efficacy data. Dose escalation may continue until the dose where at least 2 dose-limiting toxicities are observed (the MTD is exceeded). Cohort Backfill Expansion (Part 2) will allow for further characterization of safety and activity of dose levels. In Dose Expansion (Part 3), up to approximately 30 subjects will be enrolled to obtain additional safety, tolerability, PK, PD, and preliminary clinical activity data with PC-1 at a dose and schedule to be determined by the Safety Review Committee after reviewing all available safety, PK, PD, and preliminary efficacy data.

### Summary of Data and Guidance

Indications and Usage: PC-1 is in development for the treatment of mCRPC and is currently not approved for any indication.

Dosage and Administration: In the FIH study, the starting dose will be 100 μg, to be followed by dose escalation. PC-1 will be administered IV on Days 1, 8, and 15 of 21-day cycles. Subjects will be treated with 6 cycles (3 weeks each), for a total of 18 planned infusions over 18 weeks.

Preparation Instructions: Parenteral drug products should be inspected visually for particulate matter and discoloration prior to administration, whenever solution and container permit. For more details, refer to the Pharmacy Manual (or study protocol).

Dosage Forms and Strengths: The PC-1 DP will be provided as a solution for injection for IV administration at a single strength presentation of 2 mg/mL.

Contraindications: The use of PC-1 is contraindicated in the following conditions: Known hypersensitivity to any drug component.

Warnings and Precautions: There has been no prior clinical experience with PC-1. PC-1 is an antibody protein construct. Like other molecules in this class, it is highly specific for its targets. Although antibody therapeutics are well-tolerated, they are ‘foreign’ proteins, and some patients may experience infusion-related reactions (IRRs) or develop an immune response against them. However, PC-1 was designed to have fewer risks downstream of CD3 activation relative to other T cell-engaging therapies. PC-1 is an experimental drug that should be administered only to patients within the context of a clinical study.

### Potential Adverse Reactions

Because PC-1 is a T cell redirecting antibody, CRS, neurotoxicity, and/or tumor lysis syndrome may occur, although possibly at a lower frequency/severity than with other T cell activating therapies, based on the PC-1 design. Published literature of experience with other monoclonal antibody therapies indicates that pyrexia, immunogenicity reactions (ie, formation of ADAs), and/or hypersensitivity reactions may be observed. These reactions can be both serious and systemic (eg, anaphylaxis) and may occur acutely or be delayed. For regulatory reporting purposes, all adverse events (AEs) will be assessed as being unexpected at this stage of the development program. Therefore, AEs that are serious and possibly related to PC-1 will be reported to the health authorities per applicable regulations.

### Infusion-Related Reactions

PC-1 is a recombinant protein based therapeutic, and administration of therapeutic proteins has been associated with infusion reactions with symptoms and signs including fever, rigors, rash, urticaria, dyspnea, hypotension, and/or nausea. To minimize the risk of infusion reactions, all subjects will be premedicated with acetaminophen/paracetamol and diphenhydramine, and for the first infusion, corticosteroids. Mild infusion reactions should be managed per institutional standards with antipyretics, antihistamines, and antiemetics, and for more severe reactions, with corticosteroids.

### Management of Infusion-Related Reactions

IRRs are defined by National Cancer Institute (NCI) Common Terminology Criteria for Adverse Events (CTCAE) v5.0 (under the category “Injury, poisoning, and procedural complications”) as “a disorder characterized by adverse reaction to the infusion of pharmacological or biological substances”. For the purposes of this study, the time frame for infusion reaction assessment is the 24-hour period beginning from the start of the infusion. Recommendations for the management of IRRs are provided below. For Grade 1 IRR: described as mild transient reaction; infusion interruption not indicated; intervention not indicated: •Remain at bedside and monitor the subject until recovery from symptoms. For Grade 2 IRR: described as infusion interruption indicated, but responds promptly to symptomatic treatment (e.g, antihistamines, nonsteroidal anti-inflammatory drugs, narcotics, IV fluids); and prophylactic medications indicated for ≤4 hours: •Stop the PC-1 infusion, begin an IV infusion of normal saline, and treat the subject with diphenhydramine 50 mg IV (or equivalent) and/or 500 to 750 mg oral acetaminophen. •Corticosteroid therapy may also be given at the discretion of the Investigator. Remain at bedside and monitor the subject until resolution of symptoms. •If the infusion is interrupted, wait until symptoms resolve, then restart the infusion at 50% of the original infusion rate. •If no further complications occur after 60 minutes, the rate may be increased to 100% of the original infusion rate. Monitor the subject closely. If symptoms recur, stop the infusion and disconnect the subject from the infusion apparatus. •No further PC-1 will be administered at that visit. •The amount of PC-1 infused must be recorded on the case report form. •Subjects who experience a Grade 2 infusion reaction during the post-infusion observation period that does not resolve during that time should be observed until the AE resolves or stabilizes, with vital sign measurements every 4 hours and additional evaluations as medically indicated for the management of the AE.

For Grade 3 or Grade 4 IRR: Grade 3 is described as prolonged (e.g, not rapidly responsive to symptomatic medication and/or brief interruption of infusion) recurrence of symptoms following initial improvement; hospitalization indicated for other clinical sequelae (e.g, renal impairment, pulmonary infiltrates). Grade 4 is described as life-threatening consequences; urgent intervention indicated. •Immediately discontinue the infusion of PC-1. •Investigators should follow their institutional guidelines for the treatment of anaphylaxis or high-grade infusion reactions. •Consider treating the subject with an IV infusion of normal saline and administer corticosteroids, H1 and/or H2 blockers, bronchodilators, oxygen, or vasopressors as needed. •The subject should be monitored until the Investigator is comfortable that the symptoms will not recur. •All subjects with Grade ≥3 IRRs will be observed until the AEs resolve or stabilize, with vital sign measurements and additional evaluations as medically indicated for the management of the AEs. •Subjects with Grade 3 IRR during Week 1 of Cycle 1 may also be hospitalized during or after the administration of the subsequent dose, with considerations for dose reduction

### Cytokine Release Syndrome

The identified risks of treatment with T cell engagers (TCE) are primarily related to cytokine release and CRS. PC-1 is designed to reduce the risk of CRS by requiring protease cleavage for activation, focusing molecular activity to the TME where proteases are overexpressed, dysregulated, and activated. This approach has been shown to markedly reduce systemic cytokine exposure in preclinical models. Data from other clinical development programs (of non-masked TCE) has shown that broad T cell activation can lead to cytokine release into the blood, which can cause acute effects of fever, hypotension, and hypoxia and may require care in the hospital. These side effects are short-lived and can be treated with IV fluids, tocilizumab, and supportive care, but occasionally have the potential to be severe. These vital signs and adverse effects will be carefully monitored for their appearance and followed closely in this clinical study. In the nonclinical toxicology studies described in Section 4, PC-1 can be dosed at 50- to 150-fold higher doses than the unmasked active form of TCE with equivalent safety and cytokine induction activity. This safety window observed in animal studies offers the promise that higher active doses may be achieved in patients and may offer an increased level of anti-tumor activity. Guidelines for treatment of CRS have been mainly driven by the CAR-T field, where CRS seen with anti-CD19 CAR-T cells can be severe and more prolonged (Neelapu, 2018). With bispecific antibodies, CRS is shorter duration, and generally less severe, but may still be dose-limiting, and is commonly mitigated by priming/step dosing approaches. Cytokine release is primarily a first dose, or first cycle phenomenon, and tolerance to cytokine induction occurs with subsequent infusions of TCEs. Preventive measures of cytokine release and CRS will include glucocorticoid premedication, IV pre-hydration, and holding of anti-hypertensive medication on day of infusion. Priming and step dosing will be initiated if CRS is seen during dose escalation.

CRS Management: Cytokine release can lead to fevers and tachycardia and can also become more severe and lead to hypotension and hypoxia, and needs to be followed closely. CRS may also be associated with fatigue, nausea, headache, dyspnea, rigors, myalgia/arthralgia, and anorexia. CRS will be graded using the American Society for Transplantation and Cellular Therapy (ASTCT) consensus grading scale (Table 14), adapted from (Lee et al., 2019). CRS will be managed with IV hydration and other supportive care measures, as necessary, and increased monitoring. More significant CRS may require oxygen, corticosteroids, and/or tocilizumab (anti-IL-6-receptor). Severe CRS may also require intensive care unit monitoring and management with vasopressors, antiepileptics, high-dose corticosteroids, and/or mechanical ventilation. Recommended guidelines for the treatment of CRS have been mainly driven by the CAR-T field, where CRS seen with anti-CD19 CAR-T cells can be severe and more prolonged (Lee et al., 2019). With immunoglobulin G format bispecific antibodies, CRS may occur and be dose-limiting.

TABLE 14
Grading Cytokine Release Syndrome (ASTCT Consensus Scale)

| Parameter | Grade 1 | Grade 2 | Grade 3 | Grade 4 |
|---|---|---|---|---|
| Fever | ≥38° C. | ≥38° C. | ≥38° C. | ≥38° C. |
| Hypotension | None | Responds to | Requiring a | Requiring multiple |
| | | IV fluids | vasopressor | vasopressors (excluding |
| | | | | vasopressin) |
| Hypoxia | None | Requiring | Requiring | Requiring positive |
| | | low-flow nasal | high-flow nasal | pressure (eg, CPAP, |
| | | cannula | cannula, or mask | BiPAP, intubation and |
| | | | | mechanical ventilation) |

Abbreviations: ASTCT = American Society for Transplantation and Cellular Therapy; BiPAP = bilevel positive airway pressure; CPAP = continuous positive airway pressure; IV = intravenous.

For any Grade CRS:
Monitor clinical laboratory assessments.
Monitor C-reactive protein levels and serum PD cytokine laboratory assessments.
Draw extra safety laboratory samples and plasma for exploratory safety and PD biomarkers.
   For Grade 1 CRS: Many subjects may only manifest fever and tachycardia. Close monitoring is required to detect and react to early signs of progression.
Frequent monitoring of vital signs
   For Grade 2 CRS: Hypotension and/or hypoxia detected
Treat with aggressive IV hydration with normal saline
Supplemental oxygen as indicated
Consider cardiac telemetry and pulse oximetry
Consider corticosteroids (eg, dexamethasone 10 mg every 12 hours)
Consider tocilizumab or equivalent anti-IL-6 antibody (eg, siltuximab), or anti-IL-6-Receptor antibody (eg, sarilumab)

For Grade 3 CRS: Hypotension not responsive to IV fluids
Management in monitored bed or intensive care unit
Corticosteroids as indicated dexamethasone 10 mg every 6 hours, or methylprednisolone 1 mg/kg IV twice per day
Tocilizumab (4 to 8 mg/kg IV over 1 hour, not to exceed 800 mg), or equivalent anti-IL-6 antibody (eg, siltuximab), or anti-IL-6-Receptor antibody (eg, sarilumab)
Vasopressor support
Oxygen therapy with mask if needed

### Other Potential Toxicities

Tumor Lysis Syndrome; Subjects with a high disease burden may be at risk for developing tumor lysis syndrome (TLS) with PC-1 treatment. Prophylactic treatment/measures are strongly recommended for subjects considered to be at risk for TLS, per institutional or clinical standards. Subjects should be closely monitored for laboratory evidence of TLS (hyperuricemia, hyperkalemia, hyperphosphatemia, and hypocalcemia). In the case of evidence of TLS associated with PC-1, subjects will be admitted to the hospital, as clinically indicated. Standard management will include vigorous IV hydration, hypouricemic agents, and correction of acidosis, if present. Renal function, serum uric acid, calcium, phosphorus, and electrolytes should be closely monitored. Subjects with Grade 3 to 4 TLS during Week 1 or Cycle 1 may also be hospitalized for >24 hours after the end of the administration of the subsequent dose, with considerations for dose reduction as described in the study protocol.

Neurological Events: Administration of solid tumor-targeted bispecifics has shown less potential for severe neurologic toxicity (compared with CAR-T therapy or anti-CD19 BiTE format antibodies such as blinatumomab) in early-stage clinical studies. Although PC-1 at active doses has not shown a propensity for induction of cytokine levels that may be associated with neurologic toxicity in in vitro human cell cultures or in vivo cynomolgus monkey studies, the risk of neurotoxicity is unknown, and caution is warranted. Neurological events associated with cytokine release may include tremor, mental status changes, confusion, speech difficulties, and potentially seizures. Monitor subjects for neurological events and exclude other causes for neurological symptoms. Neurological events should be graded according to NCI CTCAE v5.0. Mild disorientation or expressive aphasia (trouble word-finding) may be the earliest and most specific signs. Provide supportive care as needed for any neurological events. Workup may include head magnetic resonance imaging and electroencephalogram and may require corticosteroids, or anti-seizure medications, if severe. The Medical Monitor should be contacted if there is any potentially treatment-related neurological toxicity.

### Infections

As seen with other TCE-based immunotherapies, serious infections, including fatal bacterial, fungal, and new or reactivated viral infections, may occur during and/or following the completion of PC-1-based therapy. New or reactivated viral infections may include cytomegalovirus, herpes simplex virus, parvovirus B19, varicella zoster virus, West Nile virus, hepatitis B virus (HBV), and hepatitis C virus (HCV). PC-1 should be discontinued if serious infections develop, and appropriate anti-infective therapy instituted. PC-1 is not recommended for use in subjects with severe, active infections.

Hepatitis B Reactivation: HBV reactivation can occur in patients treated with drugs classified as TCE antibodies. Cases have been reported in patients who are hepatitis B surface antigen (HBsAg)-negative but are hepatitis B core antibody (anti-HBc)-positive. HBV reactivation is defined as an abrupt increase in HBV replication manifesting as a rapid increase in serum HBV DNA levels or detection of HBsAg in a person who was previously HBsAg-negative and anti-HBc-positive. Reactivation of HBV replication is often followed by hepatitis (ie, increase in transaminase levels). In severe cases, increase in bilirubin levels, liver failure, and death can occur. It is recommended to monitor subjects with evidence of prior HBV infection (anti-HBc-positive) with HBV DNA testing monthly, and for clinical and laboratory signs of hepatitis during and for several months following PC-1 therapy.

### Immunization

The safety of immunization with live viral vaccines during or following PC-1 therapy has not been studied. Vaccination with live virus vaccines is not recommended for ≥2 weeks prior to the start of PC-1 treatment, during treatment, and until immune recovery following the last cycle of PC-1. Subjects are encouraged to get the COVID-19 vaccination/booster >2 weeks prior to starting the study. Vaccination efficacy on study is unknown; however, COVID-19 vaccination would be allowed in the study, after clearance of the first cycle, and with a 2-week dose hold.

### Other Potential Healthy Tissue Toxicities

While PSMA is most highly expressed in PC cells, and highly prostate restricted, it can be expressed at lower levels on some healthy tissues. By requiring tumor protease for activation of PC-1, there is another layer of protection for normal tissue T cell activation. Tissues would need to express PSMA, as well as express particular protease for local activation of PC-1 in non-tumor sites.

### Kidney, Gastrointestinal, and Liver

Immunohistochemistry studies have indicated that several non-prostatic tissues can express low levels of PSMA. These include kidney proximal tubule cells, and duodenal brush border cells, and rare cells colonic crypts (Silver et al., 1997; The Protein Atlas, 2022). PSMA-PET scanning, using a small molecule PSMA active site binder, has confirmed tissue accumulation in the small intestine, and liver. (Kidney accumulation cannot be resolved, as the reagent is excreted through the kidney and bladder). Renal or gastrointestinal toxicity has not been described as an issue with small molecule PSMA binders, such as 177-Lu-PSMA-617 (PLUVICTO) (Sartor et al., 2021), or with the PSMA-CD3 bispecific antibodies such as AMG160. However, transaminitis has been seen with AMG160, but has been mostly attributed to CRS (Tran et al., 2020).

Salivary and Lacrimal Glands: PSMA-PET scanning, using a small molecule PSMA active site binder, has demonstrated clear salivary and lacrimal gland uptake. It is not clear if there are other nonspecific or poorly understood mechanisms for this uptake (Morris et al., 2021; Sheehan et al., 2021). Small molecule PSMA binders, such as 177-Lu-PSMA617 (PLUVICTO), have shown a potential for a dry mouth and dry eye side effects (Sartor et al., 2021); these side effects are much less frequently seen with PSMA-CD3 bispecifics, but they can occur. (Tran et al., 2020).

Drug Interactions: The drug interaction profile of PC-1 is unknown, however, no clinically relevant drug-drug interactions would be expected with PC-1.

Use in Specific Populations

### Pregnancy

Nonclinical reproductive and developmental toxicity studies have not been conducted with PC-1. PC-1 is being developed for the treatment of progressive mCRPC, which occurs only in males. Since its impact on partner pregnancy is unknown at this time, all sexually active subjects on this study and their female partners must practice at least two highly effective protocol-specified methods of birth control while participating in clinical trials of PC-1.

### Teratogenic Effects

Whether PC-1 has teratogenic effects is unknown. Should a subject or female partner of a subject experience a pregnancy during the study, further treatment with PC-1 should be stopped, immediately.

### Nonteratogenic Effects

Whether PC-1 has nonteratogenic effects is unknown. Should a subject or female partner of a subject experience a pregnancy during the study, further treatment with PC-1 should be stopped, immediately.

### Geriatric Use

No studies in geriatric patients have been conducted. Since the average age at diagnosis of PC is 66 years (Cancer.org), elderly patients will be included in the initial clinical trials of PC-1. Elderly subjects should, however, be carefully monitored.

### Renal Impairment

The effect of renal impairment on PC-1 PK and/or function is not known. However, because PC-1 is a biologic compound, PK/functional alterations due to renal impairment are not anticipated (FDA 2020).

### Hepatic Impairment

The effect of hepatic impairment on PC-1 PK and/or function is not known.

### Overdosage

The effects of an overdose of PC-1 are not known. In the event of an overdose (ie, any dose greater than that defined in the protocol), supportive care will be provided.

### Description

PC-1 is a tri-specific TRACTr that incorporates the PSMA and CD3 binding domains, a tandem albumin-binding domain with peptide (mask) that inhibits PC-1 binding to CD3 on T cells, and a tumor protease cleavage sequence (FIG. 1). Intact PC-1 is designed to have reduced binding to CD3 on T cells (relative to unmasked versions) and minimize T cell activation and pharmacology in healthy tissue.

### Clinical Pharmacology

### Mechanism of Action

Target engagement by PC-1 requires proteolysis of its cleavable amino acid linker by proteases present in the TME. Once the amino acid cleavage sequence undergoes proteolysis by tumor proteases, the tandem mask and albumin-binding domains are released to enable CD3 binding. Simultaneous binding of bispecific components of cleaved PC-1 to CD3 and PSMA results in T cell activation in the tumor, leading to T cell-mediated killing of prostate tumor cells.

### Pharmacodynamics

Pharmacodynamics in humans have not been evaluated and will be assessed in clinical studies of PC-1.

### Pharmacokinetics

Pharmacokinetics in humans have not been previously evaluated and will be assessed in clinical studies of PC-1.

### Nonclinical Toxicology

Carcinogenesis, Mutagenesis, and Impairment of Fertility: Genotoxicity studies for biotechnology-derived pharmaceuticals are not applicable in accordance with the ICH S6(R1) guideline. Carcinogenicity, reproductive and developmental toxicity, and fertility studies have not been conducted.

Animal Toxicology and/or Pharmacology: Parameters for nonclinical safety pharmacology included cardiovascular, CNS, and respiratory safety endpoints that were incorporated into a 4-week, repeat-dose, IV toxicity study in cynomolgus monkeys (B49-0027-TX). There were no functional effects on the CNS or respiratory systems based on clinical observations of the animals and detailed weekly examinations at doses of PC-1≤1.5 mg/kg/week. Additionally, there were no clearly defined effects of PC-1 administration on the cardiovascular system of the animals based on qualitative and quantitative ECG parameters. Aside from a few episodes of non-test article-related arrhythmias, all ECGs were within normal limits.

Clinical Studies: No prior clinical studies of PC-1 have been conducted.

How Supplied/Storage and Handling: PC-1 DP will be shipped frozen and should be stored at −20° C.

### Results

Quantification of PC-1 and its Metabolites in Monkey Plasma by IC-LC-MS/MS in Preclinical Toxicokinetic Study

PC-1 is designed to masker the scFv epitope through a peptide linker with two protease cleavage sites. Enzyme-linked immunosorbent (ELISA) assay is usually standard method for the bioanalysis of biological therapeutics. It however becomes less capable in the case of BsAb PC-1s due to their very complex structures and multiple components that need to be quantified in matrix. Instead, liquid chromatography coupled with tandem mass spectrometry (LC-MS/MS) becomes a better choice owing to its features of highly specificity, multiplex platform and less-dependent on specific reagent. The results demonstrated a bioanalytical method using immunocapture (IC) followed by enzymatic digestion then LC-MS/MS analysis for this PC-1. The targets in matrix quantified included intact PC-1, its two metabolites and total antibody. The lower limit of quantification (LLOQ) was able to achieve at 5 ng/ml for metabolites. The method was successfully used to facilitate the bioanalysis of monkey plasma in preclinical TK studies.

### Methods

Immunocapture purification: Immunocapture was used to extract Intact PC-1 and its metabolites from monkey plasma. Briefly, 500 μL high capacity magnetic TM streptavidin beads was incubated with 500 μL of biotinylated human PSMA (335 ug/mL) in PBST for one hour at room temperature with shaking (1500 rpm). 100 μL plasma sample was incubated with 10.0 μL of the PSMA-coated beads prepared above for one hour at room temperature with shaking (1500 rpm). After two washing circles with 400 μL of PBST buffer, analytes enriched on the beads were eluted by 50.0 μL of 100 mM glycine-HCl (pH=2.7).

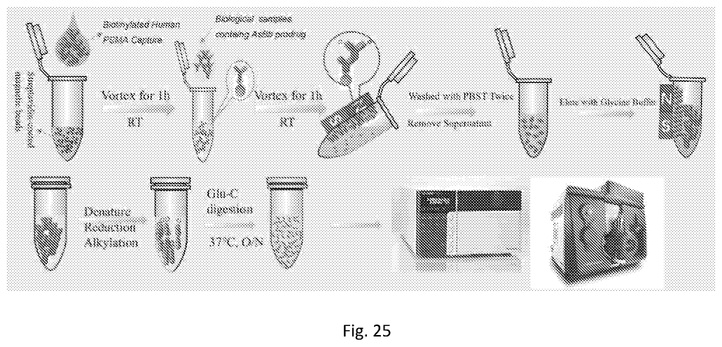

Enzymatic digestion: Take all the elution to a new 96-well plate and add stable isotopic labelled internal standard (SIL-IS) signature peptides solution. The analytes were denatured by 20 μL of 1.00 mg/mL rapigest solution, reduced by 10 μL of 100 mM dithiothreitol in 200 mM NH₄HCO₃, alkylated by 10 μL of 600 mM iodoacetamide in 200 mM NH₄HCO₃, and finally digested by 10.0 μL of 200 μg/mL Glu-C for 2 hours at 37° C. with shaking (1200 rpm). 10.0 μL of 10% trifluoroacetic acid (TFA) was used to terminate the digestion (FIG. 25).

LC-MS/MS analysis: Shimadzu-30 UPLC system coupled with AB Sciex QQQ 6500+ was used for LC-MS/MS analysis. A Waters ACQUITY UPLC HSS T3 Column (100 Å, 2.1*50 mm, 1.8 μm particle size) was used for separation. The mobile phase was 0.1% formic acid in water (MPA) and 0.1% formic acid in acetonitrile (MPB).

### Results

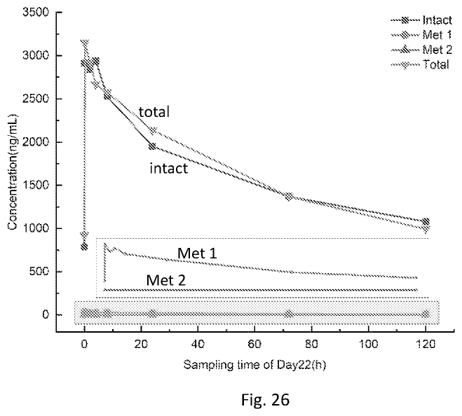

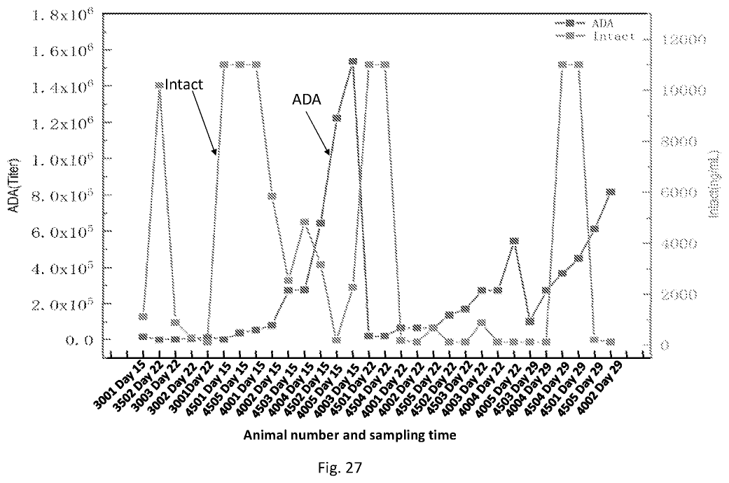

Method validation: An LC-MS/MS method for the quantitative determination for the BsAb PC-1 and its two metabolites, as well as the total antibody with the lower limit of quantifications reaching to 100, 5.00, 5.0, 110 ng/ml respectively in monkey K₂EDTA plasma has been fully validated. TK results: The concentrations of intact PC-1, its two metabolites (namely Met 1 and Met 2) and total antibody (total concentration of intact PC-1, Met 1 and Met 2) in study samples collected from the TK study have been measured. The metabolic curves for Intact PC-1, Met 1, Met 2, and Total PC-1 are shown in FIG. 26. The median Tmax values for Intact PC-1 was observed at 0.1 hours for both the first dose (Day 1) and the forth dose (Day 22) in both male and female animals at all three dose levels. In anti-drug antibody (ADA)-negative animals, the median Intact PC-1 T1/2 on Day 1 and Day 22 ranged from 67.7 to 109.1 hours across all of the doses tested. Met 1 and 2 were either below limit of quantification (BLQ) or generated during sample pretreatment instead of in vivo. The LLOQ in plasma: 5 ng/ml for Metabolite 1 and 2, 100 ng/ml for intact PC-1, and 110 ng/ml for total antibody. FIG. 27 illustrates the correlation between the intact PC-1 concentration and anti-drug antibody (ADA) concentration in the high-dose group of monkey. The metabolites of PC-1 were found to be very low in plasma of healthy monkeys, which indicated a low off-site toxicity and great advantage of on-site activation mechanism of PC-1. ADA was detected in most of middle- and high-dose animals towards the end of the dosing phase, where the exposure of prodrug was decreased significantly. The validation method is shown in Table 15.

Immunogenicity results: Intact PC-1s were detected as BLQ in a large number of samples from the high-dose group, which was probably due to a large number of ADA generated in the high-dose group (FIG. 28). The presence of ADA may cause decreased drug efficacy, thus worth paying extra attention during preclinical studies.

TABLE 15
Validation Method

| Test for Intact PC- | | |
|---|---|---|
| 1/Met1/Met2/Total | | |
| antibody | Acceptance criteria | Results |
| Standard curve: | The % DEV should be within ±20.0% | 94/93/94/95 of 96 C standard samples |
| 100/5.00/10.0/110 | (within ±25.0% at the LLOQ) | met the criteria |
| ng/mL to | for at least 75% of the C | |
| 10,000/500/500/11,000 | standards of the nominal values and | |
| ng/mL | at least 50% of the calibration | |
| | standards at each concentration | |
| | levels must fulfill these criteria. | |
| Intra-assay accuracy of | At least two-thirds of the QC | % DEV of the mean from |
| all accepted A&P runs | samples and at least half of | nominal QC concentration was |
| | replicates at each concentration | within ±20.0%/±19.6%/±12.7%/±17.1% |
| | should meet the acceptance criteria | for each QC levels. 107/111/116/114 |
| | that the mean % DEV should be | of 120 QC samples met the criteria |
| | within ±20.0% (within ±25.0% at | |
| | LLOQ) of the nominal values | |
| Intra-assay precision of | The % CV should be ≤20.0% | % CV of replicate results was within |
| all accepted A&P runs | (≤25.0% at LLOQ) | 19.7%/18.4%/12.2%/11.3% for each QC levels. |
| Inter-assay accuracy of | The mean % DEV should be | % DEV of the mean concentration |
| all accepted A&P runs | within ±20.0% (within ±25.0% | for all accepted A&P runs was |
| | at LLOQ) of the nominal values | within ±3.3%/±9.6%/±8.6%/±4.5% |
| | | of each nominal QC levels. |
| Inter-assay precision of | The % CV should be ≤20.0% | % CV of mean concentrations at each |
| all accepted A&P runs | (≤25.0% at LLOQ) | level for all accepted A&P runs was |
| | | within 18.65%/18.1% 15.1%/11.1%. |
| Specificity | The contribution of 6 individual | For Intact PC-1, Met 1 and Total PC-1: |
| | lots matrix to analyte and internal | For individual lots: in 6 of 6 individual |
| | standard should be ≤20.0% and ≤5.0% | lots, there was no interference to the |
| | respectively. | analyte and the internal standard from matrix. |
| | | For Met 2, For individual lots: in 6 of 6 |
| | | individual lots, the interference to the |
| | | analyte was within 17.3% from the |
| | | matrix. The interference to the internal |
| | | standard was within 1.1% from matrix |
| | The contribution of internal | For individual lots spiked with internal |
| | standard in 6 individual lots matrix | standard: in 6 of 6 lots, internal |
| | to analyte should be ≤20.0%. | standard had no contribution to the analyte. |
| | The mean response at the retention | For one lot plasma spiked with Met 1 |
| | of another analyte mass channel in | and Met 2 at the ULOQ concentration: |
| | the spiked samples should be no | There was no contribution from Met 1 |
| | more than 20.0% of the mean | and Met 2 to Intact PC-1 in blank matrix. |
| | LLOQ calibrator's peak signal. | For one lot plasma spiked with Met 2 |
| | | at the ULOQ concentration: There was |
| | | no contribution from Met 2 to Met 1 in |
| | | blank matrix. |
| | | For one lot plasma spiked with Intact |
| | | PC-1PC-1 at the ULOQ concentration: |
| | | The contribution of Intact PC-1PC-1 in |
| | | blank matrix to Met 2 was 51.9%. |
| | | For one lot plasma spiked with Met 1 |
| | | at the ULOQ concentration: The |
| | | contribution of Met 1 in blank matrix |
| | | to Met 2 was 182.2%. |
| | | For one lot plasma spiked with analyte |
| | | at the ULOQ concentration: there was |
| | | no contribution from analyte to the |
| | | internal standard. |
| | The contribution of analyte in blank | For one lot plasma spiked with analyte |
| | matrix to internal standard should | at the ULOQ concentration: the |
| | be ≤5.0%. | contribution from analyte to the |
| | | internal standard was 0.6%. |
| Dilution integrity for | The % DEV should be within ±20.0% | % DEV was within ±17.5%/±17.0%/±8.4%/±12.3% |
| 20 fold | of the nominal concentration for | for all diluted samples and % CV was |
| | at least two of three samples | within 8.5%/7.4%/5.8%/8.1%. |
| | and the % CV should be ≤20.0% | |
| Enzymatic Digestion | The % CV of analyte and internal | For each test level, the % CV of analyte |
| Recovery | standard at each test level and all | Enzymatic Digestion Recovery was |
| | test levels should be ≤20.0%. | within 10.2%/7.6%/10.7%/10.4%. For |
| | | all test levels, the % CV of Enzymatic |
| | | Digestion Recovery was |
| | | 16.4%/3.9%/10.2%/1.2%/7.3%. For |
| | | internal standard, the % CV of recovery |
| | | was 4.2%/6.0%/6.4%/. |
| Affinity Capture | The % CV of analyte and internal | For each test level, the % CV of analyte |
| Recovery | standard at each test level and all | affinity capture recovery was within |
| | test levels should be ≤20.0%. | 9.9%/6.9%/8.6%/6.1%/6.1%. For all |
| | | test levels, the % CV of affinity capture |
| | | recovery was within 3.1%/12.2%/10.7%/1.2%. |
| | | For internal standard, the % CV of |
| | | recovery was 5.3%/6.0%/4.8%. |
| Total Recovery | The % CV of analyte and internal | For each test level, the % CV |
| | standard at each test level and all | of analyte total recovery was |
| | test levels should be ≤20.0%. | within 9.9%/6.8%/8.5%/6.1%. |
| | | For all test levels, the % CV of total |
| | | recovery was within 19.4%/9.1%/19..9%/7.3%. |
| | | For internal standard, the % CV of recovery |
| | | was 5.2%/6.0%/4.8%. |
| Matrix effect: | % CV of IS-normalized matrix | % CV of IS-normalized matrix factor |
| for 6 lost matrix | effect for 6 lots matrix at each test | among all 6 lots was within |
| | level should be ≤20.0%. | 15.7%/15.1%/12.5%/14.2%. |
| Matrix effect in 2% | For at least two of three hemolyzed | The % DEV was |
| hemolyzed plasma | plasma samples per concentration | within ±17.6%/±20.0%/±12.7%/±19.7% |
| | level, the % DEV should be within ±20.0% | for all hemolyzed plasma samples and |
| | of the nominal values. The % CV at each | % CV of replicates at each tested level |
| | concentration level should be ≤20.0% | was within 11.0%/12.7%/6.1%/11.5%. |
| Reinjection stability at | The mean % DEV for each tested | At least 104/104/95/77 hours. |
| about 4° C. | level should be within ±20.0% and | |
| | % CV should be ≤20.0% | |
| Whole blood stability | The mean % DEV for each tested | At least 1 h on wet ice under white |
| test | level should be within ±20.0% of 0 h | light. |
| | sample and % CV should be ≤20.0% | |
| F/T cycle stability | The mean % DEV for each tested | 4/4/4 cycles |
| (Met1/Met2/Total | level should be within ±20.0% and | |
| antibody) | % CV should be ≤20.0% | |
| Bench top stability in | The mean % DEV for each tested | At least 24/24/24/24 hours at room |
| plasma | level should be within ±20.0% and | temperature under yellow light. |
| | % CV should be ≤20.0% | |
| Long term stability in | The mean % DEV for each tested | 62/62 days stored below −60° C. |
| plasma (Intact PC-1/Total | level should be within ±20.0% and | |
| antibody) | % CV should be ≤20.0% | |

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### EMBODIMENTS

Embodiment 1. An isolated recombinant polypeptide complex comprising a first chain with an amino acid sequence having at least 80% sequence identity to SEQ ID NO: 1 and a second chain with an amino acid sequence having at least 80% sequence identity to SEQ ID NO: 2 wherein the isolated recombinant polypeptide complex comprises at least one of the following characteristics:
(a) at least one disulfide bond formed by a pair of cysteine residues in the first chain or the second chain or by a pair of cysteine residues in the first chain and the second chain; or
(b) a secondary structure composition comprising a β-sheet or a random coil;
(c) at least one pyroglutamine in the second chain;
(d) a melting temperature (Tₘ) between of about 65° C. to about 85° C. when the isolated recombinant polypeptide complex is formulated at a concentration of 1.0 mg/mL in a buffer comprising 10 mM histidine buffer, 8% (w/v) Sucrose, 0.01% (w/v) polysorbate 20, pH 6.3
(e) a far UV circular dichroism peak at a wavelength between 190 nm and 205 nm when the isolated recombinant polypeptide complex is formulated at a concentration of 0.1 mg/mL in 10 mM potassium phosphate buffer pH 7.0;
(f) a far UV circular dichroism dip at a wavelength between 210 nm and 220 nm when the isolated recombinant polypeptide complex is formulated at a concentration of 0.1 mg/mL in 10 mM potassium phosphate buffer pH 7.0; or
(g) a near UV circular dichroism peak at a wavelength between 250 nm and 300 nm when the isolated recombinant polypeptide complex is formulated at a concentration of 1.0 mg/mL in a buffer comprising 10 mM histidine buffer, 8% (w/v) Sucrose, 0.01% (w/v) polysorbate 20, pH 6.3.

Embodiment 2. The isolated recombinant polypeptide complex of embodiment 1, wherein the polypeptide comprises at least two of the characteristics.

Embodiment 3. The isolated recombinant polypeptide complex of embodiment 1, wherein the polypeptide comprises at least three of the characteristics.

Embodiment 4. The isolated recombinant polypeptide complex of embodiment 1, wherein the polypeptide comprises at least four of the characteristics.

Embodiment 5. The isolated recombinant polypeptide complex of embodiment 1, wherein the polypeptide comprises at least five of the characteristics.

Embodiment 6. The isolated recombinant polypeptide complex of embodiment 1, wherein the polypeptide comprises at least six of the characteristics.

Embodiment 7. The isolated recombinant polypeptide complex of embodiment 1, wherein the polypeptide comprises at least seven of the characteristics.

Embodiment 8. The isolated recombinant polypeptide complex of embodiment 1, wherein the polypeptide comprises at least eight of the characteristics.

Embodiment 9. The isolated recombinant polypeptide complex according to any one of embodiments 1-7, wherein the first chain comprises at least 85% sequence identity to SEQ ID NO: 1.

Embodiment 10. The isolated recombinant polypeptide complex according to any one of embodiments 1-7, wherein the first chain comprises at least 90% sequence identity to SEQ ID NO: 1.

Embodiment 11. The isolated recombinant polypeptide complex according to any one of embodiments 1-7, wherein the first chain comprises at least 95% sequence identity to SEQ ID NO: 1.

Embodiment 12. The isolated recombinant polypeptide complex according to any one of embodiments 1-7, wherein the first chain comprises at least 99% sequence identity to SEQ ID NOs: 1.

Embodiment 13. The isolated recombinant polypeptide complex according to any one of embodiments 1-11, wherein the first chain comprises the amino acid sequence according to SEQ ID NO: 1.

Embodiment 14. The isolated recombinant polypeptide complex according to any one of embodiments 1-13, wherein the second chain comprises at least 85% sequence identity to SEQ ID NO: 2.

Embodiment 15. The isolated recombinant polypeptide complex according to any one of embodiments 1-13, wherein the second chain comprises at least 90% sequence identity to SEQ ID NO: 2.

Embodiment 16. The isolated recombinant polypeptide complex according to any one of embodiments 1-13, wherein the second chain comprises at least 95% sequence identity to SEQ ID NO: 2.

Embodiment 17. The isolated recombinant polypeptide complex according to any one of embodiments 1-13, wherein the second chain comprises at least 99% sequence identity to SEQ ID NO: 2.

Embodiment 18. The isolated recombinant polypeptide complex according to any one of embodiments 1-17, wherein the second chain comprises the amino acid sequence according to SEQ ID NO: 2.

Embodiment 19. The isolated recombinant polypeptide complex of embodiment 1, wherein the at least one disulfide bond is an intra-chain disulfide bond formed between Cysteine 22 and Cysteine 96, between Cysteine 138 and Cysteine 148, between Cysteine 199 and Cysteine 275, between Cysteine 339 and Cysteine 407, between Cysteine 454 and Cysteine 519, or between Cysteine 565 and Cysteine 625 of the first chain.

Embodiment 20. The isolated recombinant polypeptide complex of embodiment 1, wherein the at least one disulfide bond is an intra-chain disulfide bond formed by a pair of cysteine residues of the second chain between Cysteine 22 and Cysteine 96, or between cysteine 150 and cysteine 206 of the second chain.

Embodiment 21. The isolated recombinant polypeptide complex of embodiment 1, wherein the at least one disulfide bond is an inter-chain disulfide bond formed between the first chain and the second chain between Cysteine 645 of the first chain and Cysteine 226 of the second chain.

Embodiment 22. The isolated recombinant polypeptide complex according to any one of the above embodiments, wherein the polypeptide comprises at least two disulfide bonds formed by pairs of cysteine residues.

Embodiment 23. The isolated recombinant polypeptide complex according to any one of the above embodiments, wherein the polypeptide comprises at least three disulfide bonds formed by pairs of cysteine residues

Embodiment 24. The isolated recombinant polypeptide complex according to any one of the above embodiments, wherein the polypeptide comprises at least four disulfide bonds formed by pairs of cysteine residues

Embodiment 25. The isolated recombinant polypeptide complex according to any one of the above embodiments, wherein the polypeptide comprises at least five disulfide bonds formed by pairs of cysteine residues

Embodiment 26. The isolated recombinant polypeptide complex according to any one of the above embodiments, wherein the polypeptide comprises at least six disulfide bonds formed by pairs of cysteine residues.

Embodiment 27. The isolated recombinant polypeptide complex according to any one of the above embodiments, wherein the polypeptide comprises at least seven disulfide bonds formed by pairs of cysteine residues.

Embodiment 28. The isolated recombinant polypeptide complex according to any one of the above embodiments, wherein the polypeptide comprises at least eight disulfide bonds formed by pairs of cysteine residues.

Embodiment 29. The isolated recombinant polypeptide complex according to any one of the above embodiments, wherein the pair of cysteine residues comprises Cysteine 22 and Cysteine 96 of SEQ ID NO: 1.

Embodiment 30. The isolated recombinant polypeptide complex according to any one of the above embodiments, wherein the pair of cysteine residues comprises Cysteine 138 and Cysteine 148 of SEQ ID NO: 1.

Embodiment 31. The isolated recombinant polypeptide complex according to any one of the above embodiments, wherein the pair of cysteine residues comprises Cysteine 199 and Cysteine 275 of SEQ ID NO: 1.

Embodiment 32. The isolated recombinant polypeptide complex according to any one of the above embodiments, wherein the pair of cysteine residues comprises Cysteine 339 and Cysteine 407 of SEQ ID NO: 1.

Embodiment 33. The isolated recombinant polypeptide complex according to any one of the above embodiments, wherein the pair of cysteine residues comprises Cysteine 454 and Cysteine 519 of SEQ ID NO: 1.

Embodiment 34. The isolated recombinant polypeptide complex according to any one of the above embodiments, wherein the pair of cysteine residues comprises Cysteine 565 and Cysteine 625 of SEQ ID NO: 1.

Embodiment 35. The isolated recombinant polypeptide complex according to any one of the above embodiments, wherein the pair of cysteine residues comprises Cysteine 645 of SEQ ID NO:1 and Cysteine 226 of SEQ ID NO: 2.

Embodiment 36. The isolated recombinant polypeptide complex according to any one of the above embodiments, wherein the pair of cysteine residues comprises Cysteine 150 and Cysteine 206 of SEQ ID NO: 2.

Embodiment 37. The isolated recombinant polypeptide complex according to any one of the above embodiments, wherein the pair of cysteine residues comprises Cysteine 22 and Cysteine 96 of SEQ ID NO: 2.

Embodiment 38. The isolated recombinant polypeptide complex according to any one of the above embodiments, wherein at least one cysteine residue is a free sulfhydryl.

Embodiment 39. The isolated recombinant polypeptide complex according to any one of the above embodiments, wherein at least one cysteine residues is a free sulfhydryl, and at least one cysteine is selected from and corresponding to Cysteine 22, Cysteine 96, Cysteine 138, Cysteine 148, Cysteine 199, Cysteine 275, Cysteine 339, Cysteine 407, Cysteine 454, Cysteine 519, Cysteine 565, Cysteine 625, and Cysteine 645 of SEQ ID NO: 1.

Embodiment 40. The isolated recombinant polypeptide complex according to any one of the above embodiments, wherein at least one cysteine residues is a free sulfhydryl, and the at least one cysteine is selected from selected from and corresponding to Cysteine 22, Cysteine 96, Cysteine 150, Cysteine 226 and Cysteine 206 of SEQ ID NO: 2.

Embodiment 41. The isolated recombinant polypeptide complex according to any one of the above embodiments, wherein the at least one pair of cysteine residues comprises Cysteine 22 and Cysteine 96 of SEQ ID NO: 1, Cysteine 138 and Cysteine 148 of SEQ ID NO: 1, Cysteine 199 and Cysteine 275 of SEQ ID NO: 1, Cysteine 454 and Cysteine 519 of SEQ ID NO: 1, Cysteine 565 and Cysteine 625 of SEQ ID NO: 1, Cysteine 22 and Cysteine 96 of SEQ ID NO: 2, Cysteine 150 and Cysteine 206 of SEQ ID NO: 2, and Cysteine 645 of SEQ ID NO: 1 and Cysteine 226 of SEQ ID NO: 2.

Embodiment 42. The isolated recombinant polypeptide complex according to any one of the above embodiments wherein the secondary structure composition comprises a f-sheet and a random coil.

Embodiment 43. The isolated recombinant polypeptide complex according to any one of the above embodiments, having a melting temperature (Tm) between of about 71° C. to about 81° C.

Embodiment 44. The isolated recombinant polypeptide complex according to any one of the above embodiments, having a melting temperature (Tm) between of about 71.4° C. to about 79.5° C.

Embodiment 45. The isolated recombinant polypeptide complex according to any one of the above embodiments, having a melting temperature (Tm) between of about 65° C. to about 85° C.

Embodiment 46. The isolated recombinant polypeptide complex according to any one of the above embodiments, having a far UV circular dichroism peak at a wavelength less than or equal to 195 nm.

Embodiment 47. The isolated recombinant polypeptide complex according to any one of the above embodiments, having a far UV circular dichroism peak at a wavelength less than or equal to 205 nm.

Embodiment 48. The isolated recombinant polypeptide complex according to any one of the above embodiments, having a far UV circular dichroism dip at a wavelength less than or equal to 220 nm.

Embodiment 49. The isolated recombinant polypeptide complex according to any one of the above embodiments, having a near UV circular dichroism peak at a wavelength less than or equal to 273 nm.

Embodiment 50. The isolated recombinant polypeptide complex according to any one of the above embodiments, having a near UV circular dichroism peak at a wavelength less than or equal to 279 nm.

Embodiment 51. The isolated recombinant polypeptide complex according to any one of the above embodiments, having a near UV circular dichroism peak at a wavelength less than or equal to 290 nm.

Embodiment 52. The isolated recombinant polypeptide complex according to any one of the above embodiments, having a near UV circular dichroism peak at a wavelength less than or equal to 295 nm.

Embodiment 53. A method of treating cancer comprising administering to a subject in need thereof the isolated recombinant polypeptide complex of any one of the above embodiments.

Embodiment 54. A plurality of isolated recombinant polypeptide complexes comprising a first chain with an amino acid sequence having at least 80% sequence identity to SEQ ID NO: 1 and a second chain with an amino acid sequence having at least 80% sequence identify to SEQ ID NO: 2, wherein the plurality comprises greater than 90% monomer of the isolated recombinant polypeptide complexes.

Embodiment 55. The plurality of isolated recombinant polypeptide complexes of embodiment 54, comprising greater than 95% monomer.

Embodiment 56. The plurality of isolated recombinant polypeptide complexes of embodiment 55, comprising greater than 99% monomer.

Embodiment 57. The plurality of isolated recombinant polypeptide complexes of any one of embodiments 54-56, having a concentration greater than or equal to 1.0 mg/mL.

Embodiment 58. The plurality of recombinant polypeptides of any one of embodiment 54-57, having a concentration of at least 2.0 mg/mL.

Embodiment 59. The plurality of isolated recombinant polypeptide complexes of any one of embodiments 54-57, having a pH greater than or equal to 5.0.

Embodiment 60. The plurality of isolated recombinant polypeptide complexes of embodiment 54, comprising greater than 90% monomer at a concentration of greater than or equal to 2.0 mg/mL and a pH of 6.3.

Embodiment 61. A method for treating prostate cancer comprising administering to a subject in need thereof an antibody or antigen binding fragment that comprises a CD3 binding domain and a PSMA binding domain wherein the antibody or antibody binding fragment is administered to the subject once weekly at a dose of at least 0.1 μg/kg.

Embodiment 62. The method of embodiment 61, wherein the antibody or antigen binding fragment is administered to the subject at a dose of at least 0.1 mg/kg.

Embodiment 63. The method of any one of embodiments 61-62, wherein the antibody or antigen binding fragment is administered to the subject at a dose of at least 0.3 mg/kg.

Embodiment 64. The method of any one of embodiments 61-63, wherein the antibody or antigen binding fragment is administered to the subject at a dose of at least 1.0 mg/kg.

Embodiment 65. The method of any one of embodiments 61-64, wherein the antibody or antigen binding fragment is administered to the subject at a dose of at least 1.5 mg/kg.

Embodiment 66. The method of any one of embodiments 61-65, wherein the antibody or antigen binding fragment is administered to the subject according to the following treatment regimen: administration of the antibody or antigen binding fragment to the subject at a dose of at least 0.1 μg/kg the first week, administration of the antibody or antigen binding fragment to the subject at a dose of at least 0.3 mg/kg the second week, administration of the antibody or antigen binding fragment to the subject at a dose of at least 1.5 mg/kg the third week, followed by a 4 week non-treatment interval of the antibody or antigen binding fragment.

Embodiment 67. The isolated recombinant polypeptide complex according to any one of embodiments 1-52, wherein the isolated recombinant polypeptide complex provides a maximum plasma concentration (Cmax) in a subject within about 0.1 hour after intravenous administration.

Embodiment 68. The isolated recombinant polypeptide complex according to any one of embodiments 1-52, wherein the isolated recombinant polypeptide complex provides a Cmax of about 2500 to 3500 ng/ml in a subject within about 0.1 hour after intravenous administration of a dose of about 0.1 mg/kg.

Embodiment 69. The isolated recombinant polypeptide complex according to any one of embodiments 1-52, wherein the isolated recombinant polypeptide complex provides a Cmax of about 7500 to 10500 ng/ml in a subject within about 0.1 hour after intravenous administration of a dose of about 0.3 mg/kg.

Embodiment 70. The isolated recombinant polypeptide complex according to any one of embodiments 1-52, wherein the isolated recombinant polypeptide complex provides a Cmax of about 37500 to 52500 ng/ml in a subject within about 0.1 hour after intravenous administration of a dose of about 1.5 mg/kg.

Embodiment 71. The isolated recombinant polypeptide complex according to any one of embodiments 1-52, wherein the isolated recombinant polypeptide complex provides a half maximum plasma concentration in a subject in about 60 to about 120 hours (T1/2) after intravenous administration.

Embodiment 72. The isolated recombinant polypeptide complex of embodiment 67, wherein the value of Cmax correlates with the dose of the isolated recombinant polypeptide complex that is administered.

Embodiment 73. The isolated recombinant polypeptide complex of embodiment 72, wherein the value of Cmax is proportional to the dose of the isolated recombinant polypeptide complex that is administered.

Embodiment 74. The isolated recombinant polypeptide complex according to any one of embodiments 1-52, wherein less than 1% of the isolated recombinant polypeptide complex degrades each day in the serum of a subject.

Embodiment 75. The isolated recombinant polypeptide complex of embodiment 74, wherein the subject is diagnosed with cancer.

Embodiment 76. The isolated recombinant polypeptide complex of embodiment 75, wherein the cancer comprises prostate cancer.

Embodiment 77. The isolated recombinant polypeptide complex of embodiment 75, wherein the cancer comprises metastatic castration-resistant prostate cancer (mCRPC).

Embodiment 78. A method for treating cancer comprising administering to a subject in need thereof an effective amount of the isolated recombinant polypeptide complex according to any one of embodiments 1-52 and 67-77.

Embodiment 79. The method of embodiment 78, comprising administering the isolated recombinant polypeptide complex to the subject at a first dose and a second dose, wherein the second dose is equal to or higher than the first dose, wherein the first or second dose is at least 100 μg.

Embodiment 80. The method of embodiment 78, comprising administering the isolated recombinant polypeptide complex to the subject at a first dose, a second dose and a third dose, wherein the second dose is equal to or higher than the first dose and the third dose is equal to or higher than the second dose, wherein the first, second, or third dose is at least 100 μg.

Embodiment 81. The method of embodiment 79, wherein the method comprises a first treatment course and a second treatment course, wherein the first dose is administered to the subject in the first treatment course, wherein the second dose is administered to the subject in the second treatment course.

Embodiment 82. The method of embodiment 80, wherein the method comprises a 21-day treatment course, wherein the first dose is administered to the subject in the first week of the treatment course, wherein the second dose is administered to the subject in the second week of the treatment course, wherein the third dose is administered to the subject in the third week of the treatment course.

Embodiment 83. The method of embodiment 82, wherein the method comprises a first 21-day treatment course and a second 21-day treatment course, wherein the first dose of the second 21-day treatment course is equal to or higher than the first dose of the first 21-day treatment course, wherein the second dose of the second 21-day treatment course is equal to or higher than the second dose of the first 21-day treatment course, wherein the third dose of the second 21-day treatment course is equal to or higher than the third dose of the first 21-day treatment course.

Embodiment 84. The method of embodiment 78, wherein the method comprises a 21-day treatment cycle comprising:
(a) administering the isolated recombinant polypeptide complex to the subject at a first dose in the first week of the treatment cycle;
(b) administering the isolated recombinant polypeptide complex to the subject at a second dose in the second week of the treatment cycle; and
(c) administering the isolated recombinant polypeptide complex to the subject at a third dose in the third week of the treatment cycle.

Embodiment 85. The method of embodiment 84, wherein the method comprises:
(a) administering the isolated recombinant polypeptide complex to the subject at the first dose on the first day of the 21-day treatment cycle;
(b) administering the isolated recombinant polypeptide complex to the subject at the second dose on the eighth day of the 21-day treatment cycle; and
(c) administering the isolated recombinant polypeptide complex to the subject at the third dose on the fifteenth day of the 21-day treatment cycle.

Embodiment 86. The method of embodiment 84 or 85, wherein the treatment cycle is repeated 6 times over 18 weeks.

Embodiment 87. The method of any one of embodiments 84 to 86, wherein the first, second, or third dose is at least 100 μg.

Embodiment 88. The method of any one of embodiments 84 to 87, wherein the second dose is equal to or higher than the first dose.

Embodiment 89. The method of any one of embodiments 84 to 88, wherein the third dose is equal to or higher than the second dose.

Embodiment 90. The method of any one of embodiments 84 to 89, wherein the third dose is equal to or higher than the first dose.

Embodiment 91. The method of any one of embodiments 84 to 90, wherein the method comprises a first 21-day treatment cycle and a second 21-day treatment cycle, wherein the first dose of the second 21-day treatment cycle is equal to or higher than the first dose of the first 21-day treatment cycle, wherein the second dose of the second 21-day treatment cycle is equal to or higher than the second dose of the first 21-day treatment cycle, wherein the third dose of the second 21-day treatment cycle is equal to or higher than the third dose of the 21-day first treatment cycle.

Embodiment 92. The method of any one of embodiments 78-91, wherein the administering comprises administering through intravenous infusion.

Embodiment 93. The method of any one of embodiments 78-92, wherein the cancer comprises prostate cancer.

Embodiment 94. The method of any one of embodiments 78-92, wherein the cancer comprises mCRPC.

Embodiment 95. The method of embodiment 92, further comprising treating the subject with a therapy for an infusion-related reaction before the administering.

Embodiment 96. The method of embodiment 95, wherein the therapy for an infusion-related reaction comprises an antipyretic drug, an antihistamine drug, an antiemetic drug, or a corticosteroid.

Embodiment 97. The method of embodiment 95 or 96, wherein the therapy for an infusion-related reaction comprises acetaminophen, paracetamol, or diphenhydramine.

Embodiment 98. The method of embodiment 84, further comprising treating the subject with a corticosteroid before the step (a).

Embodiment 99. The method of embodiment 85, further comprising treating the subject with a corticosteroid before the step (a).

Embodiment 100. The method of embodiment 84, further comprising treating the subject with a therapy for cytokine release syndrome (CRS) before or after the administering.

Embodiment 101. The method of embodiment 100, wherein the therapy for CRS comprises an intravenous hydration procedure, an oxygen treatment, a corticosteroid, an immunosuppressant, a vasopressor, or an antiepileptic drug.

Embodiment 102. The method of embodiment 101, wherein the oxygen treatment comprises mechanical ventilation.

Embodiment 103. The method of embodiment 101, wherein the immunosuppressant comprises an IL-6 receptor inhibitor.

Embodiment 104. The method of embodiment 103, wherein the IL-6 receptor inhibitor comprises tocilizumab.

Embodiment 105. The method of embodiment 100, wherein the therapy for CRS comprises a high dose of a corticosteroid.

Embodiment 106. The method of embodiment 78, wherein the isolated recombinant polypeptide complex is cleaved by a tumor specific protease to generate an enzymatic product of the isolated recombinant polypeptide complex after the administering.

Embodiment 107. The method of embodiment 106, wherein the tumor specific protease comprises two or more proteases, wherein the isolated recombinant polypeptide complex is cleaved by a first protease of the two or more proteases to generate a first metabolic product of the isolated recombinant polypeptide complex, wherein the isolated recombinant polypeptide complex is cleaved by a second protease of the two or more proteases to generate a second metabolic product of the isolated recombinant polypeptide complex.

Embodiment 108. The method of embodiment 106 or 107, wherein the first protease comprises a serine protease, wherein the second protease comprises a matrix metalloprotease.

Embodiment 109. The method of embodiment 108, wherein the serine protease comprises recombinant human matriptase (MTSP1), wherein the matrix metalloprotease comprises recombinant human matrix metalloprotease 9 (MMP9).

Embodiment 120. The method of any one of embodiments 107-109, wherein the first metabolic product comprises an amino acid sequence with at least 90% sequence identity to the amino acid sequence of SEQ ID NO: 5.

Embodiment 111. The method of any one of embodiments 107-120, wherein the second metabolic product comprises an amino acid sequence with at least 90% sequence identity to the amino acid sequence of SEQ ID NO: 6.

Embodiment 112. The isolated recombinant polypeptide complex according to any one of embodiments 67-77 or the method of any one of embodiments 78-111, wherein the subject is a non-human primate or human.

Embodiment 113. The method of embodiment 112, wherein the non-human primate is cynomolgus monkey.

Embodiment 114. A pharmaceutical composition comprising: (a) the isolated recombinant polypeptide complex according to any one of embodiments 1-52 and 67-77; and (b) a pharmaceutically acceptable excipient.

Embodiment 115. The pharmaceutical composition of embodiment 114, wherein the pharmaceutically acceptable excipient comprises a buffer, a stabilizing agent, a tonicity agent, a surfactant, or combinations thereof.

Embodiment 116. The pharmaceutical composition of embodiment 115, wherein the buffer comprises an amino acid or a derivative thereof.

Embodiment 117. The pharmaceutical composition of embodiment 116, wherein the amino acid or the derivative thereof comprises L-histidine, L-histidine monohydrochloride monohydrate, or combinations thereof.

Embodiment 118. The pharmaceutical composition of embodiment 115, wherein the stabilizing agent comprises sugar.

Embodiment 119. The pharmaceutical composition of embodiment 118, wherein the sugar comprises sucrose.

Embodiment 120. The pharmaceutical composition of embodiment 115, wherein the tonicity agent comprises sugar.

Embodiment 121. The pharmaceutical composition of embodiment 120, wherein the sugar comprises sucrose.

Embodiment 122. The pharmaceutical composition of embodiment 115, wherein the surfactant comprises polysorbate 20.

Embodiment 123. The pharmaceutical composition of embodiment 117, wherein the total amount of L-histidine in the pharmaceutical composition is about 10 mM in the forms of both L-histidine and L-histidine monohydrochloride monohydrate.

Embodiment 124. The pharmaceutical composition of embodiment 123, wherein the molar ratio of L-histidine to L-histidine monohydrochloride monohydrate is about 3:2.

Embodiment 125. The pharmaceutical composition of embodiment 119 or 121, comprising about 8% (w/v) sucrose.

Embodiment 126. The pharmaceutical composition of embodiment 122, comprising at least 0.01% (w/v) polysorbate 20.

Embodiment 127. The pharmaceutical composition of any one of embodiments 114-126, comprising about 6 mM L-histidine, about 4 mM L-histidine monohydrochloride monohydrate, about 8% (w/v) sucrose, and about 0.01% (w/v) polysorbate 20.

Embodiment 128. The pharmaceutical composition of any one of embodiments 114-127, comprising about 2 mg/ml of the isolated recombinant polypeptide complex.

Embodiment 129. The pharmaceutical composition of any one of embodiments 114-128, wherein the pharmaceutical composition comprises a pH between about 5 and about 7.

Embodiment 130. The pharmaceutical composition of any one of embodiments 114-129, wherein the pharmaceutical composition comprises a pH of about 6.3.

Embodiment 131. An isolated polypeptide comprising an amino acid sequence with at least 90% sequence identity to the amino acid sequence of SEQ ID NO: 5 and wherein the isolated polypeptide is 484 amino acids in length.

Embodiment 132. An isolated polypeptide comprising an amino acid sequence with at least 90% sequence identity to the amino acid sequence of SEQ ID NO: 6 and wherein the isolated polypeptide is 476 amino acids in length.

Embodiment 133. A pharmaceutical composition comprising an isolated polypeptide comprising an amino acid sequence with at least 90% sequence identity to the amino acid sequence of SEQ ID NO: 5 and wherein the isolated polypeptide is 484 amino acids in length, and pharmaceutically acceptable excipients.

Embodiment 134. A pharmaceutical composition comprising an isolated polypeptide comprising an amino acid sequence with at least 90% sequence identity to the amino acid sequence of SEQ ID NO: 6 and wherein the isolated polypeptide is 476 amino acids in length and pharmaceutically acceptable excipients.

Embodiment 135. A method for treating cancer comprising administering to a subject in need thereof an effective amount of the pharmaceutical composition according to any one of embodiments 114-130.

Embodiment 136. The method of embodiment 135, wherein the cancer comprises mCRPC.

Embodiment 137. The method of embodiment 135, wherein the subject is human.

1. An isolated recombinant polypeptide complex comprising a first chain with an amino acid sequence having at least 80% sequence identity to SEQ ID NO: 1 and a second chain with an amino acid sequence having at least 80% sequence identity to SEQ ID NO: 2 wherein the isolated recombinant polypeptide complex comprises at least one of the following characteristics:
(a) at least one disulfide bond formed by a pair of cysteine residues in the first chain or the second chain or by a pair of cysteine residues in the first chain and the second chain;
(b) a secondary structure composition comprising a β-sheet or a random coil;
(c) at least one pyroglutamine in the second chain;
(d) a melting temperature (Tₘ) between of about 65° C. to about 85° C. when the isolated recombinant polypeptide complex is formulated at a concentration of 1.0 mg/mL in a buffer comprising 10 mM histidine buffer, 8% (w/v) Sucrose, 0.01% (w/v) polysorbate 20, pH 6.3
(e) a far UV circular dichroism peak at a wavelength between 190 nm and 205 nm when the isolated recombinant polypeptide complex is formulated at a concentration of 0.1 mg/mL in 10 mM potassium phosphate buffer pH 7.0;
(f) a far UV circular dichroism dip at a wavelength between 210 nm and 220 nm when the isolated recombinant polypeptide complex is formulated at a concentration of 0.1 mg/mL in 10 mM potassium phosphate buffer pH 7.0; or
(g) a near UV circular dichroism peak at a wavelength between 250 nm and 300 nm when the isolated recombinant polypeptide complex is formulated at a concentration of 1.0 mg/mL in a buffer comprising 10 mM histidine buffer, 8% (w/v) Sucrose, 0.01% (w/v) polysorbate 20, pH 6.3.

2.-3. (canceled)

4. The isolated recombinant polypeptide complex of claim 1, wherein the polypeptide comprises at least four of the characteristics.

5.-7. (canceled)

8. The isolated recombinant polypeptide complex of claim 1, wherein the polypeptide comprises at least eight of the characteristics.

9. (canceled)

10. The isolated recombinant polypeptide complex of claim 1, wherein the first chain comprises at least 90% sequence identity to SEQ ID NO: 1.

11.-14. (canceled)

15. The isolated recombinant polypeptide complex of claim 1, wherein the second chain comprises at least 90% sequence identity to SEQ ID NO: 2.

16.-18. (canceled)

19. The isolated recombinant polypeptide complex of claim 1, wherein the at least one disulfide bond is an intra-chain disulfide bond formed between Cysteine 22 and Cysteine 96, between Cysteine 138 and Cysteine 148, between Cysteine 199 and Cysteine 275, between Cysteine 339 and Cysteine 407, between Cysteine 454 and Cysteine 519, or between Cysteine 565 and Cysteine 625 of the first chain.

20. The isolated recombinant polypeptide complex of claim 1, wherein the at least one disulfide bond is an intra-chain disulfide bond formed by a pair of cysteine residues of the second chain between Cysteine 22 and Cysteine 96, or between cysteine 150 and cysteine 206 of the second chain.

21. The isolated recombinant polypeptide complex of claim 1, wherein the at least one disulfide bond is an inter-chain disulfide bond formed between the first chain and the second chain between Cysteine 645 of the first chain and Cysteine 226 of the second chain.

22.-23. (canceled)

24. The isolated recombinant polypeptide complex of claim 1, wherein the polypeptide comprises at least four disulfide bonds formed by pairs of cysteine residues

25.-27. (canceled)

28. The isolated recombinant polypeptide complex of claim 1, wherein the polypeptide comprises at least eight disulfide bonds formed by pairs of cysteine residues.

29.-37. (canceled)

38. The isolated recombinant polypeptide complex of claim 1, wherein at least one cysteine residue is a free sulfhydryl.

39. The isolated recombinant polypeptide complex of claim 1, wherein at least one cysteine residues is a free sulfhydryl, and at least one cysteine is selected from and corresponding to Cysteine 22, Cysteine 96, Cysteine 138, Cysteine 148, Cysteine 199, Cysteine 275, Cysteine 339, Cysteine 407, Cysteine 454, Cysteine 519, Cysteine 565, Cysteine 625, and Cysteine 645 of SEQ ID NO: 1.

40. The isolated recombinant polypeptide complex of claim 1, wherein at least one cysteine residues is a free sulfhydryl, and the at least one cysteine is selected from selected from and corresponding to Cysteine 22, Cysteine 96, Cysteine 150, Cysteine 226 and Cysteine 206 of SEQ ID NO: 2.

41. The isolated recombinant polypeptide complex of claim 1, wherein the at least one pair of cysteine residues comprises Cysteine 22 and Cysteine 96 of SEQ ID NO: 1, Cysteine 138 and Cysteine 148 of SEQ ID NO: 1, Cysteine 199 and Cysteine 275 of SEQ ID NO: 1, Cysteine 454 and Cysteine 519 of SEQ ID NO: 1, Cysteine 565 and Cysteine 625 of SEQ ID NO: 1, Cysteine 22 and Cysteine 96 of SEQ ID NO: 2, Cysteine 150 and Cysteine 206 of SEQ ID NO: 2, and Cysteine 645 of SEQ ID NO: 1 and Cysteine 226 of SEQ ID NO: 2.

42. The isolated recombinant polypeptide complex of claim 1, wherein the secondary structure composition comprises a β-sheet and a random coil.

43. (canceled)

44. The isolated recombinant polypeptide complex of claim 1, having a melting temperature (Tₘ) between of about 71.4° C. to about 79.5° C.

45. (canceled)

46. The isolated recombinant polypeptide complex of claim 1, having a far UV circular dichroism peak at a wavelength less than or equal to 195 nm.

47.-49. (canceled)

50. The isolated recombinant polypeptide complex of claim 1, having a near UV circular dichroism peak at a wavelength less than or equal to 279 nm.

51. The isolated recombinant polypeptide complex of claim 1, having a near UV circular dichroism peak at a wavelength less than or equal to 290 nm.

52. (canceled)

53. A method of treating cancer comprising administering to a subject in need thereof the isolated recombinant polypeptide complex of claim 1.

54.-137. (canceled)


SEQUENCE LISTING (19 sequences)

SEQ ID NO: 1 - protein, 645 aa, synthetic construct
EVQLVESGGGLVQPGGSLRLSCAASGSTFYTAVMGWVRQAPGKGLEWVAAIRWTALTTSYADSVKGRFTISRDGAKTTLYLQMNSLRPEDTAVYYCAARGTLGLFTTADSYDYWGQGTLVTVSSGGGGSGGGSGGVYCGPEFDESVGCMGGGGSGGGLSGRSDAGSPLGLAGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDIQMTQSPSSLSASVGDRVTITCRASQGISNYLAWYQQKTGKVPKFLIYEASTLQSGVPSRFSGGGSGTDFTLTISSLQPEDVATYYCQNYNSAPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 226 aa, synthetic construct
QVQLVESGGGVVQPGRSLRLSCAASGFAFSRYGMHWVRQAPGKGLEWVAVIWYDGSNKYYADSVKGRFTISRDNSKNTQYLQMNSLRAEDTAVYYCARGGDFLYYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 3 - protein, 468 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDIQMTQSPSSLSASVGDRVTITCRASQGISNYLAWYQQKTGKVPKFLIYEASTLQSGVPSRFSGGGSGTDFTLTISSLQPEDVATYYCQNYNSAPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 226 aa, synthetic construct; identical to SEQ ID NO: 2

SEQ ID NO: 5 - protein, 484 aa, synthetic construct
SDAGSPLGLAGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDIQMTQSPSSLSASVGDRVTITCRASQGISNYLAWYQQKTGKVPKFLIYEASTLQSGVPSRFSGGGSGTDFTLTISSLQPEDVATYYCQNYNSAPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 476 aa, synthetic construct
LAGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDIQMTQSPSSLSASVGDRVTITCRASQGISNYLAWYQQKTGKVPKFLIYEASTLQSGVPSRFSGGGSGTDFTLTISSLQPEDVATYYCQNYNSAPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 42 aa, synthetic construct
  DISULFID 3..39: note = intrachain disulfde bond
LSCAASGSTFYTAVMGWVRTTLYLQMNSLRPEDTAVYYCAAR

SEQ ID NO: 8 - protein, 62 aa, synthetic construct
GTLGLFTTADSYDYWGQGTLVTVSSGGGGSGGGSGGVYCGPEFDESVGCMGGGGSGGGLSGR

SEQ ID NO: 9 - protein, 23 aa, synthetic construct
  DISULFID 3..21: note = intrachain disulfide bond
LSCAASGFTFNKTEDTAVYYCVR

SEQ ID NO: 10 - protein, 36 aa, synthetic construct
  DISULFID 3..36: note = intrachain disulfide bond
LTCGSSTGAVTSGNYPNWVQQKLSGVQPEDEAEYYC

SEQ ID NO: 11 - protein, 48 aa, synthetic construct
  DISULFID 5..33: note = intrachain disulfide bond
VTITCRFSGGGSGTDFTLTISSLQPEDVATYYCQNYNSAPFTFGPGTK

SEQ ID NO: 12 - protein, 75 aa, synthetic construct
  DISULFID 32..60: note = intrachain disulfide bond
LTVLGGGGSDIQMTQSPSSLSASVGDRVTITCRFSGGGSGTDFTLTISSLQPEDVATYYCQNYNSAPFTFGPGTK

SEQ ID NO: 13 - protein, 33 aa, synthetic construct
  DISULFID 8..20: note = intrachain disulfide bond
SGTASVVCLLNNFYPRVYACEVTHQGLSSPVTK

SEQ ID NO: 14 - protein, 5 aa, synthetic construct
  DISULFID 3..5: note = interchain (between Chain1 and Chain2)
GECSC

SEQ ID NO: 15 - protein, 9 aa, synthetic construct
  DISULFID 7..9: note = interchain (between Chain1 and Chain2)
SFNRGECSC

SEQ ID NO: 16 - protein, 23 aa, synthetic construct
  DISULFID 3..21: note = intrachain disulfide bond
LSCAASGFAFSRAEDTAVYYCAR

SEQ ID NO: 17 - protein, 34 aa, synthetic construct
  DISULFID 3..32: note = intrachain disulfide bond
LSCAASGFAFSRNTQYLQMNSLRAEDTAVYYCAR

SEQ ID NO: 18 - protein, 72 aa, synthetic construct
  DISULFID 11..67: note = intrachain disulfide bond
STSGGTAALGCLVKDYFPEPVTVSWNSGALTSGVHTFPAVLQSSGLYSLSSVVTVPSSSLGTQTYICNVNHK

SEQ ID NO: 19 - protein, 77 aa, synthetic construct
  DISULFID 11..67: note = intrachain disulfide bond
STSGGTAALGCLVKDYFPEPVTVSWNSGALTSGVHTFPAVLQSSGLYSLSSVVTVPSSSLGTQTYICNVNHKPSNTK